SEQUENCE LISTING (105 sequences)

SEQ ID NO: 1 - protein, 206 aa, Staphylococcus aureus
MKKWTNRLMTIAGVVLILVAAYLFAKPHIDNYLHDKDKDEKIEQYDKNVKEQASKDKKQQAKPQIPKDKSKVAGYIEIPDADIKEPVYPGPATPEQLNRGVSFAEENESLDDQNISIAGHTFIDRPNYQFTNLKAAKKGSMVYFKVGNETRKYKMTSIRDVKPTDVEVLDEQKGKDKQLTLITCDDYNEKTGVWEKRKIFVATEVK

SEQ ID NO: 2 - protein, 206 aa, Staphylococcus aureus
MKKWTNRLMTIAGVVLILVAAYLFAKPHIDNYLHDKDKDEKIEQYDKNVKEQASKDKKQQAKPQIPKDKSKVAGYIEIPDADIKEPVYPGPATREQLDRGVSFVEENESLDDQNISISGHTAIDRPNYQFTNLGAAKKGSMVYFKVGNETRKYKMTSIRNVKPTAVEVLDEQKGKDKQLTLVTCDDYNVETGVWETRKIFVATEVK

SEQ ID NO: 3 - protein, 5 aa, Artificial Sequence
  Synthetic
LMVGG

SEQ ID NO: 4 - protein, 5 aa, Artificial Sequence
  Synthetic
LPESG

SEQ ID NO: 5 - protein, 5 aa, Artificial Sequence
  Synthetic
LPPAG

SEQ ID NO: 6 - protein, 4 aa, Artificial Sequence
  Synthetic
GGGK

SEQ ID NO: 7 - protein, 5 aa, Artificial Sequence
  Synthetic
GGGRR

SEQ ID NO: 8 - protein, 206 aa, Artificial Sequence
  Synthetic
MKKWTNRLMTIAGVVLILVAAYLFAKPHIDNYLHDKDKDEKIEQYDKNVKEQASKDKKQQAKPQIPKDKSKVAGYLEIPDADIKEPVYPGPATREQLDRGVCFVDEDESLDDQNISIIGHTALLRPHYQFTNLRAAKLDSIVYFTVGNETRRYKITSICNVRPTAVEVLDEHEGKDRQLTLATCDDYNYETGVWESSKIFVATEVR

SEQ ID NO: 9 - protein, 42 aa, Artificial Sequence [sequence omitted]

SEQ ID NO: 10 - protein, 43 aa, Artificial Sequence
  Synthetic
DAEFRHDSGYEVHHQKLVFFAEDVGSNKGAIIGLMVGGVVIAT

SEQ ID NO: 11 - protein, 5 aa, Artificial Sequence
  Synthetic
LMVTG

SEQ ID NO: 12 - protein, 5 aa, Artificial Sequence
  Synthetic
LPVGG

SEQ ID NO: 13 - protein, 5 aa, Artificial Sequence
  Synthetic
LAVGG

SEQ ID NO: 14 - protein, 5 aa, Artificial Sequence
  Synthetic
LPETG

SEQ ID NO: 15 - DNA, 55 bp, Artificial Sequence
  Synthetic
cccatacgacgttccagactatgcaggatctgagaacttgtactttcaaggtgct

SEQ ID NO: 16 - DNA, 60 bp, Artificial Sequence
  Synthetic
ctgttgttatcagatctcgagctattacaagtcctcttcagaaataagcttttgttcgga

SEQ ID NO: 17 - DNA, 31 bp, Artificial Sequence
  Synthetic
  misc_feature (1)..(2): n is a, c, g, or t
nnkacctgcgatgattataactttgaaaccg

SEQ ID NO: 18 - DNA, 24 bp, Artificial Sequence
  Synthetic
cagggtcagctgtttatctttgcc

SEQ ID NO: 19 - DNA, 30 bp, Artificial Sequence
  Synthetic
  misc_feature (1)..(2): n is a, c, g, or t
  misc_feature (4)..(5): n is a, c, g, or t
nnknnkaaaatttttgtggcgaccgaagtg

SEQ ID NO: 20 - DNA, 18 bp, Artificial Sequence
  Synthetic
ttcccacacgccggtttc

SEQ ID NO: 21 - DNA, 27 bp, Artificial Sequence
  Synthetic
  misc_feature (1)..(2): n is a, c, g, or t
nnkgaacagctggatcgtggcgtgagc

SEQ ID NO: 22 - DNA, 27 bp, Artificial Sequence
  Synthetic
  misc_feature (1)..(2): n is a, c, g, or t
nnkgaacagcttgatcgtggcgtgagc

SEQ ID NO: 23 - DNA, 15 bp, Artificial Sequence
  Synthetic
ggtcgccgggcccgg

SEQ ID NO: 24 - DNA, 36 bp, Artificial Sequence
  Synthetic
  misc_feature (1)..(2): n is a, c, g, or t
  misc_feature (4)..(5): n is a, c, g, or t
  misc_feature (7)..(8): n is a, c, g, or t
nnknnknnkcgtccgaactatcagtttaccaacctg

SEQ ID NO: 25 - DNA, 36 bp, Artificial Sequence
  Synthetic
  misc_feature (1)..(2): n is a, c, g, or t
  misc_feature (4)..(5): n is a, c, g, or t
  misc_feature (7)..(8): n is a, c, g, or t
nnknnknnkcgtccgtactatcagtttaccaacctg

SEQ ID NO: 26 - DNA, 30 bp, Artificial Sequence
  Synthetic
ggtatggccgataatgctaatgttctgatc

SEQ ID NO: 27 - DNA, 78 bp, Artificial Sequence
  Synthetic
cccatacgacgttccagactatgcaggatctgagaacttgtactttcaaggtgctagccaggcgagaccgcagattcc

SEQ ID NO: 28 - DNA, 78 bp, Artificial Sequence
  Synthetic
ctgttgttatcagatctcgagctattacaagtcctcttcagaaataagcttttgttcggatcctttcacttcggtcgc

SEQ ID NO: 29 - DNA, 20 bp, Artificial Sequence
  Synthetic
gtactttcaaggtgctagcc

SEQ ID NO: 30 - DNA, 21 bp, Artificial Sequence
  Synthetic
cagaaataagcttttgttatc

SEQ ID NO: 31 - protein, 6 aa, Artificial Sequence
  Synthetic
LPESGK

SEQ ID NO: 32 - DNA, 21 bp, Artificial Sequence
  Synthetic
aggcaatgcaaggagtttttg

SEQ ID NO: 33 - DNA, 33 bp, Artificial Sequence
  Synthetic
cagtgggaacaaagtcgattttgttacatctac

SEQ ID NO: 34 - DNA, 21 bp, Artificial Sequence
  Synthetic
ggcgggctatattgaaattcc

SEQ ID NO: 35 - DNA, 20 bp, Artificial Sequence
  Synthetic
  modified_base (10)..(10): ideoxyU
accgtggcgugtcctttgtg

SEQ ID NO: 36 - DNA, 23 bp, Artificial Sequence
  Synthetic
  modified_base (13)..(13): ideoxyU
aagacgaaagccuggatgatcag

SEQ ID NO: 37 - DNA, 21 bp, Artificial Sequence
  Synthetic
  modified_base (15)..(15): ideoxyU
gaaagcctggatgaucagaac

SEQ ID NO: 38 - DNA, 21 bp, Artificial Sequence
  Synthetic
  modified_base (14)..(14): ideoxyU
gtcataccgcgctucttcgtc

SEQ ID NO: 39 - DNA, 22 bp, Artificial Sequence
  Synthetic
  modified_base (11)..(11): ideoxyU
actatcagttuaccaacctgag

SEQ ID NO: 40 - DNA, 23 bp, Artificial Sequence
  Synthetic
taccgcgcttgaccgtccgcact

SEQ ID NO: 41 - DNA, 24 bp, Artificial Sequence
  Synthetic
  modified_base (12)..(12): ideoxyU
aactatcagttuaccaacctgagg

SEQ ID NO: 42 - DNA, 21 bp, Artificial Sequence
  Synthetic
  modified_base (16)..(16): ideoxyU
cgaaactagacagcaucgtgt

SEQ ID NO: 43 - DNA, 23 bp, Artificial Sequence
  Synthetic
ggcggcgaaaaaagacagcatcg

SEQ ID NO: 44 - DNA, 21 bp, Artificial Sequence
  Synthetic
  modified_base (15)..(15): ideoxyU
agcatcgtgtatttuacagtg

SEQ ID NO: 45 - DNA, 30 bp, Artificial Sequence
  Synthetic
actagacagcatggtgtattttacagtggg

SEQ ID NO: 46 - DNA, 31 bp, Artificial Sequence
  Synthetic
  modified_base (17)..(17): ideoxyU
atcgtgtattttaaagugggcaacgaaaccc

SEQ ID NO: 47 - DNA, 28 bp, Artificial Sequence
  Synthetic
cgaaacccgtaagtataaaataaccagc

SEQ ID NO: 48 - DNA, 21 bp, Artificial Sequence
  Synthetic
  modified_base (16)..(16): ideoxyU
ccagcatttgtaacgugagac

SEQ ID NO: 49 - DNA, 25 bp, Artificial Sequence
  Synthetic
  modified_base (14)..(14): ideoxyU
agcattcgtaacgugagaccgaccg

SEQ ID NO: 50 - DNA, 22 bp, Artificial Sequence
  Synthetic
  modified_base (14)..(14): ideoxyU
accgcggtggaagugctggatg

SEQ ID NO: 51 - DNA, 25 bp, Artificial Sequence
  Synthetic
  modified_base (14)..(14): ideoxyU
aggaaggcaaagauagacagctgac

SEQ ID NO: 52 - DNA, 25 bp, Artificial Sequence
  Synthetic
  modified_base (14)..(14): ideoxyU
ataaaggcaaagauagacagctgac

SEQ ID NO: 53 - DNA, 23 bp, Artificial Sequence
  Synthetic
aggcaaagataaacagctgaccc

SEQ ID NO: 54 - DNA, 22 bp, Artificial Sequence
  Synthetic
  modified_base (13)..(13): ideoxyU
acctgcgatgatuataactatg

SEQ ID NO: 55 - DNA, 22 bp, Artificial Sequence
  Synthetic
  modified_base (12)..(12): ideoxyU
aaaccggcgtgugggaatccag

SEQ ID NO: 56 - DNA, 26 bp, Artificial Sequence
  Synthetic
cgtgtgggaaactagtaaaatttttg

SEQ ID NO: 57 - DNA, 26 bp, Artificial Sequence
  Synthetic
gtgggaatcccgtaaaatttttgtgg

SEQ ID NO: 58 - DNA, 35 bp, Artificial Sequence
  Synthetic
  modified_base (16)..(16): ideoxyU
accgaagtgaaaggauccgaacaaaagcttatttc

SEQ ID NO: 59 - DNA, 20 bp, Artificial Sequence
  Synthetic
actttgcttttatctttcgg

SEQ ID NO: 60 - DNA, 20 bp, Artificial Sequence
  Synthetic
  modified_base (10)..(10): ideoxyU
acgccacggucgagctgttc

SEQ ID NO: 61 - DNA, 32 bp, Artificial Sequence
  Synthetic
  modified_base (13)..(13): ideoxyU
aggctttcgtctucttccacaaagcacacgcc

SEQ ID NO: 62 - DNA, 34 bp, Artificial Sequence
  Synthetic
  modified_base (14)..(14): ideoxyU
atcatccaggcttucgttttcgtccacaaagcac

SEQ ID NO: 63 - DNA, 43 bp, Artificial Sequence
  Synthetic
  modified_base (11)..(11): ideoxyU
aagcgcggtaugaccggaaatgctaatgttctgatcatccagg

SEQ ID NO: 64 - DNA, 39 bp, Artificial Sequence
  Synthetic
  modified_base (13)..(13): ideoxyU
gtaaactgatagugcggacgaagaatcgcggtatgaccg

SEQ ID NO: 65 - DNA, 23 bp, Artificial Sequence
  Synthetic
tgaccgataatgctaatgttctg

SEQ ID NO: 66 - DNA, 28 bp, Artificial Sequence
  Synthetic
  modified_base (12)..(12): ideoxyU
aaactgatagtucggacgaagaagcgcg

SEQ ID NO: 67 - DNA, 46 bp, Artificial Sequence
  Synthetic
  modified_base (15)..(15): ideoxyU
gatgctgtctagttucgccgcccccaggttggtaaactgatagtgc

SEQ ID NO: 68 - DNA, 21 bp, Artificial Sequence
  Synthetic
ctcaggttggtaaactgatag

SEQ ID NO: 69 - DNA, 34 bp, Artificial Sequence
  Synthetic
  modified_base (17)..(17): ideoxyU
gtaaaatacacgatgcugcctagtttcgccgccc

SEQ ID NO: 70 - DNA, 18 bp, Artificial Sequence
  Synthetic
ttcgccgccctcaggttg

SEQ ID NO: 71 - DNA, 31 bp, Artificial Sequence
  Synthetic
  modified_base (17)..(17): ideoxyU
actttaaaatacacgaugctgtctagtttcg

SEQ ID NO: 72 - DNA, 18 bp, Artificial Sequence
  Synthetic
ttgcccactgtaaaatac

SEQ ID NO: 73 - DNA, 38 bp, Artificial Sequence
  Synthetic
  modified_base (14)..(14): ideoxyU
acgttacaaatgcuggtcattttatatctacgggtttc

SEQ ID NO: 74 - DNA, 34 bp, Artificial Sequence
  Synthetic
  modified_base (14)..(14): ideoxyU
acgttacgaatgcuggttattttatatctacggg

SEQ ID NO: 75 - DNA, 37 bp, Artificial Sequence
  Synthetic
  modified_base (15)..(15): ideoxyU
cacttccaccgcggucggtttcacgttacaaatgctg

SEQ ID NO: 76 - DNA, 33 bp, Artificial Sequence
  Synthetic
  modified_base (14)..(14): ideoxyU
atctttgccttccugttcatccagcacttccac

SEQ ID NO: 77 - DNA, 33 bp, Artificial Sequence
  Synthetic
  modified_base (14)..(14): ideoxyU
atctttgcctttaugttcatccagcacttccac

SEQ ID NO: 78 - DNA, 21 bp, Artificial Sequence
  Synthetic
tcatgttcatccagcacttcc

SEQ ID NO: 79 - DNA, 34 bp, Artificial Sequence
  Synthetic
  modified_base (13)..(13): ideoxyU
aatcatcgcaggugaccagggtcagctgtctatc

SEQ ID NO: 80 - DNA, 37 bp, Artificial Sequence
  Synthetic
  modified_base (12)..(12): ideoxyU
acacgccggttuctacgttataatcatcgcaggtcgc

SEQ ID NO: 81 - DNA, 20 bp, Artificial Sequence
  Synthetic
ccggtttcatagttataatc

SEQ ID NO: 82 - DNA, 23 bp, Artificial Sequence
  Synthetic
acgccggtttcatagttataatc

SEQ ID NO: 83 - DNA, 22 bp, Artificial Sequence
  Synthetic
  modified_base (16)..(16): ideoxyU
atcctttcacttcggucgccac

SEQ ID NO: 84 - DNA, 50 bp, Artificial Sequence
  Synthetic
  modified_base (12)..(12): ideoxyU
atcgtccgaacuatcagtttaccaacctgcgcgcggcgaaaaaaggcagc

SEQ ID NO: 85 - DNA, 45 bp, Artificial Sequence
  Synthetic
  modified_base (10)..(10): ideoxyU
agggtcagcugtctatctttgcctttctgttcatccagcacttcc

SEQ ID NO: 86 - DNA, 42 bp, Artificial Sequence
  Synthetic
  modified_base (10)..(10): ideoxyU
agctgacccuggcgacctgcgatgattataacgtggaaaccg

SEQ ID NO: 87 - DNA, 57 bp, Artificial Sequence
  Synthetic
  modified_base (12)..(12): ideoxyU
agttcggacgaucaatcgcggtatggccgataatgctaatgttctgatcatccaggc

SEQ ID NO: 88 - DNA, 34 bp, Artificial Sequence
  Synthetic
  modified_base (17)..(17): ideoxyU
accagcatttgtaacgugaaaccgaccgcggtgg

SEQ ID NO: 89 - DNA, 36 bp, Artificial Sequence
  Synthetic
  modified_base (17)..(17): ideoxyU
aaaaattttactggttucccacacgccggtttccac

SEQ ID NO: 90 - DNA, 41 bp, Artificial Sequence
  Synthetic
  modified_base (17)..(17): ideoxyU
aaaccagtaaaattttugtggcgaccgaagtgaaaggatcc

SEQ ID NO: 91 - DNA, 43 bp, Artificial Sequence
  Synthetic
  modified_base (17)..(17): ideoxyU
acgttacaaatgctggucattttatatttacgggtttcgttgc

SEQ ID NO: 92 - DNA, 25 bp, Artificial Sequence
  Synthetic
cgccgttaataggagctcgatccgg

SEQ ID NO: 93 - DNA, 20 bp, Artificial Sequence
  Synthetic
cccaccgccaccaaccatca

SEQ ID NO: 94 - protein, 7 aa, Artificial Sequence
  Synthetic
GLPVGGV

SEQ ID NO: 95 - protein, 7 aa, Artificial Sequence
  Synthetic
GLMVGGV

SEQ ID NO: 96 - protein, 7 aa, Artificial Sequence
  Synthetic
GLPESGT

SEQ ID NO: 97 - protein, 6 aa, Artificial Sequence
  Synthetic
LMVTGV

SEQ ID NO: 98 - protein, 6 aa, Artificial Sequence
  Synthetic
LPVGGV

SEQ ID NO: 99 - protein, 7 aa, Artificial Sequence
  Synthetic
ALAVGGS

SEQ ID NO: 100 - protein, 7 aa, Artificial Sequence
  Synthetic
ALPPAGS

SEQ ID NO: 101 - protein, 5 aa, Artificial Sequence
  Synthetic
GVVIA

SEQ ID NO: 102 - protein, 4 aa, Artificial Sequence
  Synthetic
GGRR

SEQ ID NO: 103 - protein, 6 aa, Artificial Sequence
  Synthetic
LMVGGK

SEQ ID NO: 104 - protein, 5 aa, Artificial Sequence
  Synthetic
  misc_feature (3)..(3): Xaa can be any naturally occurring amino acid
LPXTG

SEQ ID NO: 105 - protein, 4 aa, Artificial Sequence
  Synthetic
GGGH

(19) United States
(12) Patent Application Publication
(10) Pub. No.: US 2024/0077502 A1
(43) Pub. Date: Mar. 7, 2024

(54) AMYLOID PROTEIN MODIFYING SORTASES AND USES THEREOF

(71) Applicants: The Broad Institute, Inc., Cambridge, MA (US); President and Fellows of Harvard College, Cambridge, MA (US); The Brigham and Women's Hospital Inc., Boston, MA (US)

(72) Inventors: David R. Liu, Cambridge, MA (US); Christopher J Podracky, Cambridge, MA (US); Chihui An, Cambridge, MA (US); Dominic Walsh, Boston, MA (US)

(73) Assignees: The Broad Institute, Inc., Cambridge, MA (US); President and Fellows of Harvard College, Cambridge, MA (US); The Brigham and Women's Hospital Inc., Boston, MA (US)

(21) Appl. No.: 18/271,650

(22) PCT Filed: Jan. 11, 2022
(86) PCT No.: PCT/US2022/011897

§ 371 Date: Jul. 10, 2023

Related U.S. Application Data
(60) Provisional application No. 63/136,186, filed on Jan. 11, 2021.

(51) Int. Cl. G01N 33/68; C12N 9/52; G01N 33/533; G01N 33/573

(52) U.S. Cl. CPC G01N 33/6896; C12N 9/52; C12Y 304/2207; G01N 33/533; G01N 33/573; G01N 2333/4709; G01N 2333/952

(57) ABSTRACT

Evolved sortases exhibiting enhanced reaction kinetics and/or altered substrate preferences are provided herein, for example evolved sortases that bind recognitions motifs comprising a LMVGG [SEQ ID NO: 3] sequence. Also provided are methods (e.g., orthogonal transpeptidation and diagnostics methods) for using such sortases.

### RELATED APPLICATIONS

This application claims priority under 35 U.S.C. § 119(e) to U.S. Provisional Application Ser. No. 63/136,186, filed Jan. 11, 2021, the entire contents of which are incorporated herein by reference.

### FEDERALLY SPONSORED RESEARCH

This invention was made with government support under EB022376, GM118062, and AG046275 awarded by the National Institutes of Health. The government has certain rights in the invention.

### BACKGROUND

The spectrum of bond-forming reactions catalyzed by naturally occurring enzymes, e.g., naturally occurring sortases, ligases, polymerases, and kinases, is limited and typically restricted to specific substrates. Such enzymes can be used to form bonds between molecules, e.g., proteins, nucleic acids, carbohydrates, or small molecules, under physiological conditions, thus allowing in vivo and in vitro modification of molecules in or on living cells and other biological structures while maintaining their structural integrity. For example, sortases catalyze a transpeptidation reaction that results in the conjugation of a peptide comprising a C-terminal sortase recognition motif with a peptide comprising an N-terminal sortase recognition motif. Naturally occurring sortases are typically selective for specific C-terminal and N-terminal recognition motifs, e.g., LPXTG [SEQ ID NO: 104] (where X represents any amino acid) and GGG, respectively. The T and the G in the substrate can be connected using a peptide bond or an ester linkage. The spectrum of peptides and proteins that can be conjugated via sortases is, therefore, limited. While target proteins not comprising a sortase recognition sequence may be engineered to add such a sequence, such engineering is often cumbersome or impractical, e.g., in situations where the addition of an exogenous sortase recognition motif would disturb the structure and/or the function of the native protein. Another obstacle to a broader application of bond-forming enzymes to biological systems is that naturally occurring bond-forming enzymes typically exhibit low reaction efficiencies. The generation of bond-forming enzymes that efficiently catalyze bond-forming reactions and/or utilize a different, non-natural target substrate, e.g., a desired sortase recognition sequence, would allow for a broader use of sortases to modify proteins in research, therapeutic, and diagnostic application.

### SUMMARY

Epitope-specific enzymes are powerful tools for site-specific protein modification, but generally require genetic manipulation of the target protein. Here, laboratory evolution of the bacterial transpeptidase sortase A to recognize the LMVGG [SEQ ID NO: 3] sequence in endogenous A3 protein is described. Using a yeast display selection for covalent bond formation, a sortase was evolved that prefers LMVGG [SEQ ID NO: 3] substrates from a starting enzyme that prefers LPESG [SEQ ID NO: 4] substrates (e.g., as represented in SEQ ID NO: 2), representing a >1,400-fold change in substrate preference. This evolved sortase was used to label endogenous Aβ in human cerebrospinal fluid (CSF), enabling detection of Aβ with sensitivities rivaling those of commercial assays. The evolved sortase can conjugate a hydrophilic peptide to Aβ42, greatly impeding the ability of the resulting protein to aggregate into higher-order structures. In some embodiments, evolved sortases described herein are useful for site-specific protein modification (e.g., sortase-mediated labeling of Aβ) without target gene manipulation. In some embodiments, evolved sortases described herein are useful for inhibiting aggregation of Aβ proteins.

Accordingly, in some aspects, the disclosure provides a sortase that binds substrates comprising the amino acid sequence LMVGG [SEQ ID NO: 3], wherein the sortase comprises an amino acid sequence that is at least 80% identical to the amino acid sequence provided in SEQ ID NO: 2, or a fragment thereof, wherein the amino acid sequence of the sortase includes one or more substitutions selected from the group consisting of the amino acid substitutions listed in Table 3, relative to SEQ ID NO: 2.

In some embodiments, the sortase comprises at least three mutations, at least four mutations, at least five, at least six, at least seven, at least eight, at least nine, or at least 10 amino acid substitutions as compared to the amino acid sequence set forth in SEQ ID NO: 2 or a fragment thereof. In some embodiments, the evolved sortase sequence comprises up to 40 amino acid substitutions as compared to the amino acid sequence set forth in SEQ ID NO: 2 or a fragment thereof. In some embodiments, the evolved sortase sequence comprises 1, 2, 3, 4, 5, 6, 7, 8, 9, 10, 11, 12, 13, 14, 15, 16, 17, 18, 19, 20, 21, 22, 23, 24, 25, or more amino acid substitutions (e.g., mutations) as compared to the amino acid sequence set forth in SEQ ID NO: 2 or a fragment thereof, selected from the group consisting of the amino acid substitutions listed in Table 3.

In some embodiments, the sortase comprises amino acid substitutions at two or more of the following positions: I76, S102, E105, N107, S118, I123, D124, N127, G134, K138, G139, M141, K145, K152, M155, R159, K162, Q172, K173, K177, V182, V189, T196, R197, and K206, relative to SEQ ID NO: 2.

In some embodiments, the sortase comprises the following amino acid substitutions relative to SEQ ID NO: 2: S118I, G134R, R159C, K177R, V182A, and R197S.

In some embodiments, the sortase comprises the following amino acid substitutions relative to SEQ ID NO: 2: I76L, S102C, E105D, N107D, S118I, I123L, D124L, N127H, G134R, K138L, G139D, M141I, K145T, K152R, M155I, R159C, K162R, Q172H, K173E, K177R, V182A, V189Y, T196S, R197S, and K206R.

In some embodiments, the sortase has reduced selectivity for peptides having the amino acid sequence LPPAG [SEQ ID NO: 5] relative to the sortase set forth in SEQ ID NO: 2.

In some embodiments, the sortase has increased selectivity for peptides having the amino acid sequence LMVGG [SEQ ID NO: 3] relative to the sortase set forth in SEQ ID NO: 2.

In some embodiments, the sortase has 10-fold to 100-fold preference for LMVGG [SEQ ID NO: 3] over LPESG [SEQ ID NO: 4].

In some embodiments, the sortase has a change in substrate preference of at least 1,400-fold to favor LMVGG [SEQ ID NO: 3] over LPESG [SEQ ID NO: 4].

In some embodiments, the sortase modifies (e.g., transpeptidates) an Alzheimer's disease-associated amyloid β-protein (Aβ). In some embodiments, the modifying comprises conjugating a heterologous peptide to the amyloid β-protein (Aβ).

In some embodiments, the amyloid β-protein (Aβ) comprises between 30 and 51 amino acids. In some embodiments, the amyloid β-protein (Aβ) comprises between 40 and 42 amino acids.

In some embodiments, the sortase is active in human plasma.

In some aspects, the disclosure provides a method for producing a sortase protein variant, the method comprising: expressing in a population of yeast cells one or more fusion proteins, each fusion protein comprising a sortase protein or portion thereof conjugated to a triglycine peptide having an N-terminus capable of reacting in sortase-catalyzed reactions; incubating the yeast cell population with a mixture comprising N-terminally biotinylated target substrates and non-biotinylated off-target substrates under conditions under which the sortases expressed by the yeast catalyze transpeptidation of the biotinylated target substrates to the surface of the yeast cells; treating the yeast cells with a TEV protease; incubating the cells with fluorescently-labeled streptavidin under conditions under which the streptavidin binds to the biotin on the surface of the yeast cells comprising the target substrate; and isolating the fluorescently-labeled yeast cells form the population of yeast cells using fluorescence-activated cell sorting (FACS).

In some embodiments, the sortase of the fusion protein comprises the amino acid sequence set forth in SEQ ID NO: 2.

In some embodiments, the target substrate comprises the amino acid sequence LMVGG [SEQ ID NO: 3].

In some embodiments, the incubating occurs in human plasma.

In some aspects, the disclosure provides a method for detecting a target protein in a biological sample, the method comprising contacting a biological sample with a sortase as described herein, and a probe comprising one or more detectable agents; and a peptide comprising the amino acid sequence GGGK [SEQ ID NO: 6] under conditions under which the sortase conjugates the one or more detectable agents to the target protein; removing unconjugated probe from the biological sample; and detecting the presence of the detectable agent conjugated to the target protein.

In some embodiments, the target protein comprises the amino acid sequence LMVGG [SEQ ID NO: 3]. In some embodiments, the target protein is amyloid β-protein (Aβ).

In some embodiments, the biological sample comprises cerebrospinal fluid (CSF).

In some embodiments, the detectable agent comprises biotin. In some embodiments, the biotin comprises a fluorescent label.

In some aspects, the disclosure provides a method for inhibiting amyloid β-protein (Aβ) aggregation or plaque formation in a cell or subject, the method comprising administering to the cell or subject a sortase as described herein and a peptide comprising the amino acid sequence GGGRR [SEQ ID NO: 7]. In some embodiments, the GGGRR [SEQ ID NO: 7] is at the N-terminus of the peptide.

In some embodiments, the cell is a human cell. In some embodiments, the subject is a human. In some embodiments, the cell is a central nervous system cell. In some embodiments, the cell is a neuron. In some embodiments, the subject has or is suspected of having Alzheimer's disease.

In some embodiments, the amyloid β-protein (Aβ) comprises between 30 and 51 amino acids. In some embodiments, the amyloid β-protein (Aβ) comprises between 40 and 42 amino acids.

In some aspects, the disclosure provides a method for treating or ameliorating Alzheimer's disease (AD) in a subject, the method comprising administering to a subject having AD a sortase as described herein and a peptide comprising the amino acid sequence GGGRR [SEQ ID NO: 7].

In some embodiments, the subject is a human.

In some embodiments, the GGGRR [SEQ ID NO: 7] is at the N-terminus of the peptide.

In some embodiments, Aβ aggregation or plaque formation in a cell is inhibited.

### BRIEF DESCRIPTION OF DRAWINGS

FIGS. 1A-1D show evolved sortase activity on fetuin A. FIG. 1A: Fetuin A (5 μM) was incubated with SrtA 8.5-H3 (20 μM) and GGGK[SEQ ID NO: 6](Btn) (100 μM). Labeled fetuin A is detected by Western blot. The laddering in lane 4 is only observed in the presence of both SrtA and fetuin. FIG. 1B: Western blot of overnight reaction of SrtA 8.5-H3 (50 μM) and GGGK[SEQ ID NO: 6](Btn) (1 mM) in human plasma shows labeling of endogenous fetuin A. FIG. 1C: In a two hour reaction of SrtAβ (1 μM) and GGGK[SEQ ID NO: 6](Btn) (1 mM) in human plasma, no enzyme dependent modifications are observed upon streptavidin pulldown and Coomassie staining. The bands observed in the +SrtAβ +GGGK[SEQ ID NO: 6](Btn) lane are also observed in the GGGK[SEQ ID NO: 6](Btn) only lane. Notably, treatment with sortase 4S.6 under the same conditions leads to pulldown of a protein not observed in the other lanes. FIG. 1D: Western blot of these reactions prior to pulldown shows that 4S.6, but not SrtAβ, labels fetuin A. This is notable evidence of a change in substrate specificity between 4S.6 and SrtAβ. Labeling of purified fetuin A, plasma Western blot, and plasma pulldown were each performed three times with similar results.

FIG. 2 shows one embodiment of a yeast display strategy for sortase evolution. A population of yeast displays a library of ˜107 SrtA variants. 1. Triglycine is conjugated to the surface of each cell with Sfp phosphopathetheinyl transferase. 2. The cells are incubated with biotinylated target substrate and non-biotinylated off-target substrates. 3. After allowing the SrtA variants to catalyze transpeptidation between triglycine and the added substrates, cells are washed and the SrtA variants are removed from their surfaces using TEV protease. Cells are labelled with an anti-HA antibody to quantify sortase expression and streptavidin-PE to quantify transpeptidation between triglycine and the positive selection substrate. Active sortase variants have higher on-target transpeptidation per unit expression than inactive variants or promiscuous variants and can be isolated by fluorescence activated cell sorting (FACS). 4. Collected cells were grown, reinduced, and further enriched for target recognition.

FIG. 3 shows TEV treatment reduces SrtA display to uninduced levels. TEV cleavage to remove SrtA also removes its C-terminal c-Myc tag. Staining of an induced population of cells, cells from that same population that have been treated with TEV as described above, and uninduced cells for c-Myc tag (chicken anti-c-myc, Invitrogen A-21281, followed by goat anti-chicken IgY AlexaFluor 488 conjugate, Invitrogen A-11039) shows that TEV treatment reduces the amount of apparent c-Myc to uninduced levels.

FIG. 4 shows mutational analysis of the round 7 consensus sequence. Single mutant reversions from the round 7 consensus sequence (R7) were made back to 4S.6 and wild-type SrtA. After induction and preparation for cell surface sortase reactions, clonal populations were incubated for 1 hour with 100-1000 nM Btn-LMVGG[SEQ ID NO: 3]. Reversion mutant Y94R showed a 2.3-fold improvement on average across substrate concentrations compared to the round 7 consensus sequence. L123I (1.5-fold) and L124D (1.1-fold) also showed improvement. These three residues and residue 122 (by virtue of its proximity to 123 and 124) were targeted for site-saturation mutagenesis heading into round 9. Activity is defined as fold-increase in PE signal over a negative control (0 nM Btn-LMVGG[SEQ ID NO: 3]) aliquot of each variant.

FIGS. 5A-5D show activity profile and mutational analysis of SrtAβ. FIG. 5A: The evolved SrtAβ and the starting enzyme 4S.6 were displayed on yeast and assayed for their ability to catalyze transpeptidation on different substrates. SEQ ID NOs: 3 (LMVGG), 12 (LPVGG), 5 (LPPAG), and 4 (LPESG) are shown. FIG. 5B: SrtAβ, 4S.6, and all 25 single reversion mutants were displayed on yeast and assayed for their ability to catalyze transpeptidation between triglycine and Btn-LMVGG[SEQ ID NO: 3]. Reversion mutants with activity less than half that of SrtAβ are highlighted in dark gray. FIG. 5C: The predicted location of the six reversion mutations that reduce SrtAβ by >50% are shown on the NMR solution structure of wild-type S. aureus SrtA (PDB: 2KID). An LPXTG [SEQ ID NO: 104] substrate analogue, and calcium ion required for activity are shown. Residues 118, 182, and 197 are part of the substrate binding pocket, while other residues are further from the active site. FIG. 5D: The activity of 4S.6, a minimal mutant (4S.6 with S118I, G134R, R159C, K177R, V182A, and R197S mutations), and SrtAβ on Btn-LMVGG[SEQ ID NO: 3] were compared by flow cytometry. Addition of these six mutations to 4S.6 improves activity on the LMVGG [SEQ ID NO: 3] substrate, but is insufficient to confer the level of activity displayed by SrtAβ, highlighting the importance of other mutations. All graphs represent the mean of three replicates±standard deviation. Activity is defined as the ratio of cell surface biotinylation (PE) to sortase expression level (FITC).

FIG. 6 shows calcium dependence of evolved SrtAβ. Several mutations in SrtAβ map near the calcium binding site. To assess the impact of these mutations on sortase calcium dependence, 20 μM Abz-LMVGG[SEQ ID NO: 3](Dnp)-CONH2 was treated with SrtAβ in the presence of varying concentrations of calcium. Samples containing calcium showed an increase in fluorescence over time, while samples lacking calcium failed to rise above the level of a negative control lacking enzyme. Notably, SrtAβ shows activity at physiologically relevant calcium concentrations (typical ionized calcium levels in plasma range from 1.3-1.5 mM).

FIGS. 7A-7B show sortase labeling of endogenous Aβ in human cerebrospinal fluid. FIG. 7A: Transpeptidation of Aβ40 or Aβ42 with GGG should yield Aβ37-GGG, which is not detected by Aβ40 and Aβ42-specific ELISAs. Treatment of CSF specimens with SrtAβ and GGG caused a significant reduction in ELISA-measured levels of both Aβ40 and Aβ42. SEQ ID NO: 101 (GVVIA) is shown. FIG. 7B: Transpeptidation of Aβ40 or Aβ42 with GGGK[SEQ ID NO: 6](Btn) yields Aβ37-GGGK[SEQ ID NO: 6](Btn), which can be detected through its affinity handle. Detectable levels of transpeptidation product are observed in all 10 CSF samples. Importantly, no product was observed (* indicates below limit of detection) in the absence of SrtAβ or GGGK[SEQ ID NO: 6](Btn). Product yield is defined as the amount of product detected divided by the amount of Aβ40+Aβ42 measured in each sample. For each labeling experiment, all reactions were set up in triplicate. Bars represent the mean of three replicates±standard deviation. The GGG labeling experiment was performed once. The GGGK[SEQ ID NO: 6](Btn) labeling experiment was performed twice. The data presented are representative of both attempts. SEQ ID NO: 101 (GVVIA) is also shown.

FIGS. 8A-8D show HPLC traces of semi-syntheses and other reaction mixtures. FIG. 8A: In a representative injection from the AβM1-37GGGK[SEQ ID NO: 6](Btn) semi-synthesis described above, 82% of the starting AβM1-40 was converted to the desired product, with no clear evidence of hydrolysis or alternate transpeptidation products. FIG. 8B: 120 μM chemically synthesized Aβ40 (0.25 mg scale) was reacted overnight with 40 μM SrtAβ and 1 mM of the indicated glycine-based nucleophile before lyophilization, dissolution in 7 M guanidium chloride, 50 mM Tris pH 7.5, 2 mM EDTA, and analysis of the crude reaction mixture by HPLC. In the presence of SrtAβ but the absence of glycine nucleophiles, a peak was observed that does not fully resolve from the enzyme or Aβ40. This putative hydrolysis product has an area roughly one-quarter that of the Aβ40 peak. In the presence of glycine nucleophiles this product is never observed. Instead, the expected transpeptidation products were observed in yields of 80-88%. SEQ ID NOs: 6 (GGGK) and 105 (GGGH) are shown. FIG. 8C: In a representative injection from the AβM1-37GGGRR[SEQ ID NO: 7] semi-synthesis described above, 64% of the starting AβM1-42 was converted to the desired product, with no clear evidence of hydrolysis or alternate transpeptidation products. FIG. 8D: 120 μM chemically synthesized Aβ42 (0.4 mg scale) was reacted overnight with 30 μM SrtAβ and 1 mM or 200 μM GGGRR [SEQ ID NO: 7] before lyophilization, dissolution in 7 M guanidium chloride, 50 mM Tris pH 7.5, 2 mM EDTA, and analysis of the crude reaction mixture by HPLC using the column and protocol from the AβM1-37GGGK[SEQ ID NO: 6](Btn) semi-synthesis. 64% of the Aβ42 was converted to the expected product when reacted with 1 mM GGGRR [SEQ ID NO: 7] as opposed to 50% when reacted with 200 μM GGGRR [SEQ ID NO: 7].

FIG. 9 shows aggregation inhibition of Aβ42 with SrtAβ. Thioflavin T binding was used to monitor the aggregation of AβM1-42 and AβM1-37GGGRR[SEQ ID NO: 7]. Data points from the time-course are shown for each replicate (n=3 for AβM1-42, n=4 for 10 μM Aβ(M1-37GGGRR[SEQ ID NO: 7]), and n=6 for 20 μM Aβ(M1-37GGGRR[SEQ ID NO: 7])) and curves fitted to each replicate by Boltzmann equation are indicated. The initiation of aggregation of Aβ(M1-37GGGRR[SEQ ID NO: 7]) monomer was greatly retarded compared to AβM1-42, with an average t1/2=14.6 hours at 20 μM and 28.3 hours at 10 μM (compared to 0.6 hours and 0.7 hours for AβM1-42 at 20 μM and 10 μM, respectively). SEQ ID NO: 101 (GVVIA) is also shown.

### DEFINITIONS

The term “agent,” as used herein, refers to any molecule, entity, or moiety. For example, an agent may be a protein, an amino acid, a peptide, a polynucleotide, a carbohydrate, a lipid, a detectable label, a binding agent, a tag, a metal atom, a contrast agent, a catalyst, a non-polypeptide polymer, a synthetic polymer, a recognition element, a linker, or chemical compound, such as a small molecule. In some embodiments, the agent is a binding agent, for example, a ligand, a ligand-binding molecule, an antibody, or an antibody fragment. In some embodiments, the term “modifying agent” is used interchangeably with “agent.” Additional agents suitable for use in embodiments of the present invention will be apparent to the skilled artisan. The invention is not limited in this respect.

The term “amino acid,” as used herein, includes any naturally occurring and non-naturally occurring amino acid. Suitable natural and non-natural amino acids will be apparent to the skilled artisan, and include, but are not limited to, those described in S. Hunt, The Non-Protein Amino Acids: In Chemistry and Biochemistry of the Amino Acids, edited by G. C. Barrett, Chapman and Hall, 1985. Some non-limiting examples of non-natural amino acids are 4-hydroxyproline, desmosine, gamma-aminobutyric acid, beta-cyanoalanine, norvaline, 4-(E)-butenyl-4(R)-methyl-N-methyl-L-threonine, N-methyl-L-leucine, 1-amino-cyclopropanecarboxylic acid, 1-amino-2-phenyl-cyclopropanecarboxylic acid, 1-amino-cyclobutanecarboxylic acid, 4-amino-cyclopentenecarboxylic acid, 3-amino-cyclohexanecarboxylic acid, 4-piperidylacetic acid, 4-amino-1-methylpyrrole-2-carboxylic acid, 2,4-diaminobutyric acid, 2,3-diaminopropionic acid, 2,4-diaminobutyric acid, 2-aminoheptanedioic acid, 4-(aminomethyl)benzoic acid, 4-aminobenzoic acid, ortho-, meta- and para-substituted phenylalanines (e.g., substituted with —C(═O)C₆H₅; —CF₃; —CN; -halo; —NO₂; —CH₃), disubstituted phenylalanines, substituted tyrosines (e.g., further substituted with —C(═O)C₆H₅; —CF₃; —CN; -halo; —NO₂; —CH₃), and statine. In the context of amino acid sequences, “X” or “Xaa” represents any amino acid residue, e.g., any naturally occurring and/or any non-naturally occurring amino acid residue.

The term “amyloid β-protein” also referred to as “Aβ”, as used herein, refers to an Alzheimer's disease-associated protein. The formation of Aβ plaque deposits in the central nervous system is the hallmark of Alzheimer's disease (AD). Like other neurodegenerative disease-related proteins, Aβ proteins self-assemble into aggregates. In some embodiments, evolved sortases provided herein are useful for inhibiting aggregation of Aβ proteins. In some embodiments, the amyloid β-protein (Aβ) comprises between 20 and 100 amino acids (e.g., 20, 30, 40, 50, 60, 70, 80, 90, or 100). In some embodiments, the amyloid β-protein (Aβ) comprises between 20 and 75, between 30 and 51, between 40 and 45 amino acids. In some embodiments, the amyloid β-protein (Aβ) comprises between 40 and 42 amino acids. Aβ protein sequence is contemplated. Non-limiting exemplary amyloid β-proteins (Aβ) comprise the amino acid sequence

[SEQ ID NO: 9]
DAEFRHDSGYEVHHQKLVFFAEDVGSNKGAIIGLMVGGVVIA,
or
[SEQ ID NO: 10]
DAEFRHDSGYEVHHQKLVFFAEDVGSNKGAIIGLMVGGVVIAT.

Aβ monomers are predominantly extracellular, unstructured and contain a five-amino-acid sequence (LMVGG [SEQ ID NO: 3] at residues 34-38). In some embodiments, an evolved sortase provided herein mediates the covalent modification of Aβ peptides. In some embodiments, the evolved sortase provided herein is an S. aureus sortase A (e.g., SrtAβ). In certain embodiments, a sortase variant, SrtAβ, provided herein, is used to biotinylate and detect endogenous Aβ in clinical cerebrospinal fluid samples (CSF). In some embodiments, SrtAβ-mediated conjugation of a hydrophilic pentapeptide to Aβ42 greatly slows the initiation of detectable aggregation.

The term “antibody,” as used herein, refers to a protein belonging to the immunoglobulin superfamily. The terms antibody and immunoglobulin are used interchangeably. Antibodies from any mammalian species (e.g., human, mouse, rat, goat, pig, horse, cattle, camel) and from non-mammalian species (e.g., from non-mammalian vertebrates, birds, reptiles, amphibia) are within the scope of the term. Suitable antibodies and antibody fragments for use in the context of some embodiments of the present invention include, for example, human antibodies, humanized antibodies, domain antibodies, F(ab′), F(ab′)2, Fab, Fv, Fc, and Fd fragments, antibodies in which the Fc and/or FR and/or CDR1 and/or CDR2 and/or light chain CDR3 regions have been replaced by homologous human or non-human sequences; antibodies in which the FR and/or CDR1 and/or CDR2 and/or light chain CDR3 regions have been replaced by homologous human or non-human sequences; antibodies in which the FR and/or CDR1 and/or CDR2 and/or light chain CDR3 regions have been replaced by homologous human or non-human sequences; and antibodies in which the FR and/or CDR1 and/or CDR2 regions have been replaced by homologous human or non-human sequences. In some embodiments, so-called single chain antibodies (e.g., ScFv), (single) domain antibodies, and other intracellular antibodies may be used in the context of the present invention. Domain antibodies, camelid and camelized antibodies and fragments thereof, for example, VHH domains, or nanobodies, such as those described in patents and published patent applications of Ablynx NV and Domantis are also encompassed in the term antibody. Further, chimeric antibodies, e.g., antibodies comprising two antigen-binding domains that bind to different antigens, are also suitable for use in the context of some embodiments of the present invention.

The term “binding agent,” as used herein refers to any molecule that binds another molecule. In some embodiments, a binding agent binds another molecule with high affinity. In some embodiments, a binding agent binds another molecule with high specificity. The binding agent may be a protein, peptide, nucleic acid, carbohydrate, polymer, or small molecule. Examples for binding agents include, without limitation, antibodies, antibody fragments, receptors, ligands, aptamers, receptors, and adnectins.

The term “bond-forming enzyme,” as used herein, refers to any enzyme that catalyzes a reaction resulting in the formation of a covalent bond. In some embodiments, the bond-forming enzyme is a sortase.

The term “conjugated” or “conjugation” refers to an association of two entities, for example, of two molecules such as two proteins, or a protein and a reactive handle, or a protein and an agent, e.g., a detectable label. The association can be, for example, via a direct or indirect (e.g., via a linker) covalent linkage or via non-covalent interactions. In some embodiments, the association is covalent. In some embodiments, two molecules are conjugated via a linker connecting both molecules. For example, in some embodiments where two proteins are conjugated to each other to form a protein fusion, the two proteins may be conjugated via a polypeptide linker, e.g., an amino acid sequence connecting the C-terminus of one protein to the N-terminus of the other protein. In some embodiments, conjugation of a protein to a protein or peptide is achieved by transpeptidation using a sortase. See, e.g., Ploegh et al., International PCT Patent Application, PCT/US2010/000274, filed Feb. 1, 2010, published as WO/2010/087994 on Aug. 5, 2010, Ploegh et al., International Patent Application PCT/US2011/033303, filed Apr. 20, 2011, published as WO/2011/133704 on Oct. 27, 2011, Chaikof et al., U.S. Provisional Patent Application, U.S. Ser. No. 61/720,294, filed Oct. 30, 2012, and Liu et al., U.S. patent application U.S. Ser. No. 13/922,812, filed Jun. 20, 2013 the entire contents of each of which are incorporated herein by reference, for exemplary sortases, proteins, recognition motifs, reagents, and methods for sortase-mediated transpeptidation.

The term “detectable label” refers to a moiety that has at least one element, isotope, or functional group incorporated into the moiety which enables detection of the molecule, e.g., a protein or peptide, or other entity, to which the label is attached. Labels can be directly attached or can be attached via a linker. It will be appreciated that the label may be attached to or incorporated into a molecule, for example, a protein, polypeptide, or other entity, at any position. In general, a detectable label can fall into any one (or more) of five classes: I) a label which contains isotopic moieties, which may be radioactive or heavy isotopes, including, but not limited to, ²H, ³H, ¹³C, ¹⁴C, ¹⁵N, ¹⁸F, ³¹P, ³²P, 35S, ⁶⁷Ga, ⁷⁶Br, ^{99m}Tc (Tc-⁹⁹m), ¹¹¹In, ¹²³I, ¹²⁵I, ¹³¹I, ¹⁵³Gd, ¹⁶⁹Yb, and ¹⁸⁶Re; II) a label which contains an immune moiety, which may be antibodies or antigens, which may be bound to enzymes (e.g., such as horseradish peroxidase); III) a label which is a colored, luminescent, phosphorescent, or fluorescent moieties (e.g., such as the fluorescent label fluorescein-isothiocyanate (FITC); IV) a label which has one or more photo affinity moieties; and V) a label which is a ligand for one or more known binding partners (e.g., biotin-streptavidin, FK506-FKBP). In certain embodiments, a label comprises a radioactive isotope, preferably an isotope which emits detectable particles, such as β particles. In certain embodiments, the label comprises a fluorescent moiety. In certain embodiments, the label is the fluorescent label fluorescein-isothiocyanate (FITC). In certain embodiments, the label comprises a ligand moiety with one or more known binding partners. In certain embodiments, the label comprises biotin, which may be detected using a streptavidin conjugate (e.g., fluorescent streptavidin conjugates such as Streptavidin ALEXA FLUOR® 568 conjugate (SA-568) and Streptavidin ALEXA FLUOR® 800 conjugate (SA-800), Invitrogen). In some embodiments, a label is a fluorescent polypeptide (e.g., GFP or a derivative thereof such as enhanced GFP (EGFP)) or a luciferase (e.g., a firefly, Renilla, or Gaussia luciferase). It will be appreciated that, in certain embodiments, a label may react with a suitable substrate (e.g., a luciferin) to generate a detectable signal. Non-limiting examples of fluorescent proteins include GFP and derivatives thereof, proteins comprising fluorophores that emit light of different colors such as red, yellow, and cyan fluorescent proteins. Exemplary fluorescent proteins include, e.g., Sirius, Azurite, EBFP2, TagBFP, mTurquoise, ECFP, Cerulean, TagCFP, mTFP1, mUkGl, mAG1, AcGFP1, TagGFP2, EGFP, mWasabi, EmGFP, TagYPF, EYFP, Topaz, SYFP2, Venus, Citrine, mKO, mKO2, mOrange, mOrange2, TagRFP, TagRFP-T, mStrawberry, mRuby, mCherry, mRaspberry, mKate2, mPlum, mNeptune, T-Sapphire, mAmetrine, mKeima. See, e.g., Chalfie, M. and Kain, SR (eds.) Green fluorescent protein: properties, applications, and protocols Methods of biochemical analysis, v. 47 Wiley-Interscience, Hoboken, N.J., 2006; and Chudakov, D M, et al., Physiol Rev. 90(3):1103-63, 2010, for discussion of GFP and numerous other fluorescent or luminescent proteins. In some embodiments, a label comprises a dark quencher, e.g., a substance that absorbs excitation energy from a fluorophore and dissipates the energy as heat.

The term “fetuin A”, as used herein, refers to a protein, also referred to as alpha-2-HS-glycoprotein (AHSG), that is abundant in plasma (e.g., human plasma). In some embodiments, a starting sortase provided herein is capable of modifying fetuin A in human plasma. In some embodiments, fetuin A comprises a native LPPAG [SEQ ID NO: 5], which is recognized by the starting sortase (e.g. 4S.6, represented by SEQ ID NO: 2). In some embodiments, evolution with negative selection against the LPPAG [SEQ ID NO: 5] sequence of fetuin is performed. In some embodiments, the evolved sortases provided herein have reduced selectivity for peptides having the amino acid sequence LPPAG [SEQ ID NO: 5] relative to the sortase set forth in SEQ ID NO: 2. In some embodiments, the evolved sortases provided herein have reduced activity on fetuin A relative to the sortase set forth in SEQ ID NO: 2.

The term “homologous”, as used herein is an art understood term that refers to nucleic acids or polypeptides that are highly related at the level of nucleotide or amino acid sequence. Nucleic acids or polypeptides that are homologous to each other are termed “homologues.” Homology between two sequences can be determined by sequence alignment methods known to those of skill in the art. For example, the homology, or “percent identity” of two amino acid sequences can be determined using the algorithm of Karlin and Altschul Proc. Natl. Acad. Sci. USA 87:2264-68, 1990, modified as in Karlin and Altschul Proc. Natl. Acad. Sci. USA 90:5873-77, 1993. Such an algorithm is incorporated into the NBLAST and XBLAST programs (version 2.0) of Altschul, et al. J. Mol. Biol. 215:403-10, 1990. BLAST protein searches can be performed with the XBLAST program, score=50, wordlength=3 to obtain amino acid sequences homologous to the protein molecules of interest. Where gaps exist between two sequences, Gapped BLAST can be utilized as described in Altschul et al., Nucleic Acids Res. 25(17):3389-3402, 1997. When utilizing BLAST and Gapped BLAST programs, the default parameters of the respective programs (e.g., XBLAST and NBLAST) can be used. In accordance with the invention, two sequences are considered to be homologous if they are at least about 50-60% identical, e.g., share identical residues (e.g., amino acid residues) in at least about 50-60% of all residues comprised in one or the other sequence, at least about 70% identical, at least about 80% identical, at least about 90% identical, at least about 95% identical, at least about 98% identical, at least about 99% identical, at least about 99.5% identical, or at least about 99.9% identical, for at least one stretch of at least 20, at least 30, at least 40, at least 50, at least 60, at least 70, at least 80, at least 90, at least 100, at least 120, at least 150, or at least 200 amino acids.

The term “k_{cat}” refers to the turnover rate of an enzyme, e.g., the number of substrate molecules that the respective enzyme converts to product per time unit. Typically, k_{cat} designates the turnover of an enzyme working at maximum efficiency.

The term “K_{M}” is used herein interchangeably with the term “Kₘ” and refers to the Michaelis constant of an enzyme, an art-recognized measure designating the substrate concentration at ½ the maximum reaction velocity of a reaction catalyzed by the respective enzyme.

The term “linker,” as used herein, refers to a chemical group or molecule covalently linked to a molecule, for example, a protein, and a chemical group or moiety, for example, a click chemistry handle. In some embodiments, the linker is positioned between, or flanked by, two groups, molecules, or other moieties and connected to each one via a covalent bond, thus connecting the two. In some embodiments, the linker is an amino acid or a plurality of amino acids (e.g., a peptide or protein). In some embodiments, the linker is an organic molecule, group, polymer (e.g., PEG), or chemical moiety.

The term “mutation,” as used herein, refers to a substitution of a residue within a sequence, e.g., a nucleic acid or amino acid sequence, with another residue, or a deletion or insertion of one or more residues within a sequence. Mutations are typically described herein by identifying the original residue followed by the position of the residue within the sequence and by the identity of the newly substituted residue. For example, the term “P94S” in the context of describing a mutation in the S. aureus sortase A protein describes a mutation in which the P (proline) residue at position 94 in the sortase A sequence has been replaced by an S (serine) residue, the term “P94R” describes a mutation in which the P (proline) residue at position 94 in the sortase A sequence has been replaced by an R (arginine) residue, the term “E106G” describes a mutation in which the E (glutamate) residue at position 106 in the sortase A sequence has been replaced by a G (glycine) residue, and so forth. See, e.g., SEQ ID NO: 1 for reference of the respective amino acid residue positions in the wild-type S. aureus sortase A protein. It should be appreciated that methods for making the amino acid substitutions (mutations) provided herein are well known in the art, and are provided by, for example, Green and Sambrook, Molecular Cloning: A Laboratory Manual (4^{th} ed., Cold Spring Harbor Laboratory Press, Cold Spring Harbor, N.Y. (2012)).

The “percent identity” of two amino acid sequences may be determined using algorithms or computer programs, for example, the algorithm of Karlin and Altschul, Proc. Natl. Acad. Sci. USA 87:2264-68, 1990, modified as in Karlin and Altschul, Proc. Natl. Acad. Sci. USA 90:5873-77, 1993. Such an algorithm is incorporated into various computer programs, for example NBLAST and XBLAST programs (version 2.0) of Altschul et al. J. Mol. Biol. 215:403-10, 1990. BLAST protein searches can be performed with the XBLAST program, score=50, word length=3 to obtain amino acid sequences homologous to the protein molecules of interest. Where gaps exist between two sequences, Gapped BLAST can be utilized as described in Altschul et al., Nucleic Acids Res. 25(17):3389-3402, 1997. When utilizing BLAST and Gapped BLAST programs, the default parameters of the respective programs (e.g., XBLAST and NBLAST) can be used. BLAST nucleotide searches can be performed with the NBLAST nucleotide program parameters set, e.g., for score=100, wordlength=12 to obtain nucleotide sequences homologous to a nucleic acid molecule described herein. BLAST protein searches can be performed with the XBLAST program parameters set, e.g., to score 50, wordlength=3 to obtain amino acid sequences homologous to a protein molecule described herein. To obtain gapped alignments for comparison purposes, Gapped BLAST can be utilized as described in Altschul, S F et al., (1997) Nuc. Acids Res. 25: 3389 3402. Alternatively, PSI BLAST can be used to perform an iterated search which detects distant relationships between molecules (Id.). When utilizing BLAST, Gapped BLAST, and PSI Blast programs, the default parameters of the respective programs (e.g., of XBLAST and NBLAST) can be used (see, e.g., National Center for Biotechnology Information (NCBI) on the worldwide web, ncbi.nlm.nih.gov). Another specific, non-limiting example of a mathematical algorithm utilized for the comparison of sequences is the algorithm of Myers and Miller, 1988, CABIOS 4:11 17. Such an algorithm is incorporated in the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. The percent identity between two sequences can be determined using techniques similar to those described above, with or without allowing gaps. In calculating percent identity, typically only exact matches are counted.

The terms “protein,” “peptide,” and “polypeptide” are used interchangeably herein, and refer to a polymer of amino acid residues linked together by peptide (amide) bonds. The terms refer to a protein, peptide, or polypeptide of any size, structure, or function. Typically, a protein, peptide, or polypeptide will be at least three amino acids long. A protein, peptide, or polypeptide may refer to an individual protein or a collection of proteins. One or more of the amino acids in a protein, peptide, or polypeptide may be modified, for example, by the addition of a chemical entity such as a carbohydrate group, a hydroxyl group, a phosphate group, a farnesyl group, an isofarnesyl group, a fatty acid group, a linker for conjugation, functionalization, or other modification, etc. A protein, peptide, or polypeptide may also be a single molecule or may be a multi-molecular complex. A protein, peptide, or polypeptide may be just a fragment of a naturally occurring protein or peptide. A protein, peptide, or polypeptide may be naturally occurring, recombinant, or synthetic, or any combination thereof.

The term “small molecule” is used herein to refer to molecules, whether naturally-occurring or artificially created (e.g., via chemical synthesis) that have a relatively low molecular weight. Typically, a small molecule is an organic compound (i.e., it contains carbon). A small molecule may contain multiple carbon-carbon bonds, stereocenters, and other functional groups (e.g., amines, hydroxyl, carbonyls, or heterocyclic rings). In some embodiments, small molecules are monomeric and have a molecular weight of less than about 1500 g/mol. In certain embodiments, the molecular weight of the small molecule is less than about 1000 g/mol or less than about 500 g/mol. In certain embodiments, the small molecule is a drug, for example, a drug that has already been deemed safe and effective for use in humans or animals by the appropriate governmental agency or regulatory body.

The term “sortase,” as used herein, refers to a protein having sortase activity, i.e., an enzyme able to carry out a transpeptidation reaction conjugating the C-terminus of a protein (or the C-terminus of a peptide conjugate, i.e., an agent comprising a peptide) to the N-terminus of a protein (or the N-terminus of a peptide conjugate, i.e., an agent comprising a peptide) via transamidation. The term includes full-length sortase proteins, e.g., full-length naturally occurring sortase proteins, fragments of such sortase proteins that have sortase activity, modified (e.g., mutated) variants or derivatives of such sortase proteins or fragments thereof, as well as proteins that are not derived from a naturally occurring sortase protein, but exhibit sortase activity. Those of skill in the art will readily be able to determine whether or not a given protein or protein fragment exhibits sortase activity, e.g., by contacting the protein or protein fragment in question with a suitable sortase substrate under conditions allowing transpeptidation and determining whether the respective transpeptidation reaction product is formed. In some embodiments, a sortase is a protein comprising at least 20 amino acid residues, at least 30 amino acid residues, at least 40 amino acid residues, at least 50 amino acid residues, at least 60 amino acid residues, at least 70 amino acid residues, at least 80 amino acid residues, at least 90 amino acid residues, at least 100 amino acid residues, at least 125 amino acid residues, at least 150 amino acid residues, at least 175 amino acid residues, at least 200 amino acid residues, or at least 250 amino acid residues. In some embodiments, a sortase is a protein comprising less than 100 amino acid residues, less than 125 amino acid residues, less than 150 amino acid residues, less than 175 amino acid residues, less than 200 amino acid residues, or less than 250 amino acid residues. In some embodiments, the sortase comprises a sortase catalytic domain and, optionally, an additional domain, e.g., a transmembrane domain.

Suitable sortases will be apparent to those of skill in the art and include, but are not limited to, sortase A, sortase B, sortase C, and sortase D type sortases. Suitable sortases are described, for example, in Dramsi S, Trieu-Cuot P, Bierne H, Sorting sortases: a nomenclature proposal for the various sortases of Gram-positive bacteria. Res Microbiol. 156(3):289-97, 2005; Comfort D, Clubb RT. A comparative genome analysis identifies distinct sorting pathways in gram-positive bacteria. Infect Immun., 72(5):2710-22, 2004; Chen I, Dorr BM, and Liu DR., A general strategy for the evolution of bond-forming enzymes using yeast display. Proc Natl Acad Sci USA. 2011 Jul. 12; 108(28):11399; and Pallen, M. J.; Lam, A. C.; Antonio, M.; Dunbar, K. TRENDS in Microbiology, 2001, 9(3), 97-101; the entire contents of each of which are incorporated herein by reference). Amino acid sequences of sortases and the nucleotide sequences that encode them are known to those of skill in the art and are disclosed in a number of references cited herein, the entire contents of all of which are incorporated herein by reference. Those of skill in the art will appreciate that any sortase and any sortase recognition motif can be used in some embodiments of this invention, including, but not limited to, the sortases and sortase recognition motifs described in Ploegh et al., International PCT Patent Application, PCT/US2010/000274, filed Feb. 1, 2010, published as WO/2010/087994 on Aug. 5, 2010; Ploegh et al., International Patent Application PCT/US2011/033303, filed Apr. 20, 2011, published as WO/2011/133704 on Oct. 27, 2011; Liu et al., U.S. Pat. No. 9,267,127, issued Feb. 23, 2016; and Liu et al., U.S. Pat. No. 10,202,593, issued Feb. 12, 2009, the entire contents of each of which are incorporated herein by reference.

In some embodiments, the sortase is sortase A of S. aureus. The amino acid sequence of wild-type sortase A of S. aureus is known to those of skill in the art, and a representative sequence (gil21284177|ref|NP_647265.1) is provided below:

[SEQ ID NO: 1]
MKKWTNRLMTIAGVVLILVAAYLFAKPHIDNYLHDKDKDEKIE
QYDKNVKEQASKDKKQQAKPQIPKDKSKVAGYIEIPDADIKEP
VYPGPATPEQLNRGVSFAEENESLDDQNISIAGHTFIDRPNYQ
FTNLKAAKKGSMVYFKVGNETRKYKMTSIRDVKPTDVEVLDEQ
KGKDKQLTLITCDDYNEKTGVWEKRKIFVATEVK.

In some embodiments, a starting sortase (e.g., sortase 4S.6 comprising the amino acid sequence set forth in SEQ ID NO: 2, provided below), is derived from wild-type S. aureus sortase A.

[SEQ ID NO: 2]
MKKWTNRLMTIAGVVLILVAAYLFAKPHIDNYLHDKDKDEKIE
QYDKNVKEQASKDKKQQAKPQIPKDKSKVAGYIEIPDADIKEP
VYPGPATREQLDRGVSFVEENESLDDQNISISGHTAIDRPNYQ
FTNLGAAKKGSMVYFKVGNETRKYKMTSIRNVKPTAVEVLDEQ
KGKDKQLTLVTCDDYNVETGVWETRKIFVATEVK.

In some embodiments, sortase 4S.6 [SEQ ID NO: 2], serves as the starting sortase for generating the evolved sortases and their methods of use disclosed herein. In some embodiments, an evolved sortase provided herein comprises one or more substitutions selected from the group consisting of the amino acid substitutions listed in Table 3, relative to SEQ ID NO: 2. In some embodiments, the evolved sortase comprises or consists of an amino acid sequence set forth in SEQ ID NO: 8, provided below:

[SEQ ID NO: 8]
MKKWTNRLMTIAGVVLILVAAYLFAKPHIDNYLHDKDKDEKIE
QYDKNVKEQASKDKKQQAKPQIPKDKSKVAGYLEIPDADIKEP
VYPGPATREQLDRGVCFVDEDESLDDQNISIIGHTALLRPHYQ
FTNLRAAKLDSIVYFTVGNETRRYKITSICNVRPTAVEVLDEH
EGKDRQLTLATCDDYNYETGVWESSKIFVATEVR.

Additional S. aureus sortase A sequences will be apparent to those of skill in the art, and the invention is not limited in this respect. In some embodiments, the sortase is a sortase A of another organism, for example, from another bacterial strain, such as S. pyogenes. In some embodiments, the sortase is a sortase B, a sortase C, or a sortase D. Suitable sortases from other bacterial strains will be apparent to those of skill in the art, and the invention is not limited in this respect.

The term “sortase substrate,” as used herein refers to a molecule or entity that can be utilized in a sortase-mediated transpeptidation reaction. Typically, a sortase utilizes two substrates—a substrate comprising a C-terminal sortase recognition motif, and a second substrate comprising an N-terminal sortase recognition motif and the transpeptidation reaction results in a conjugation of both substrates via a covalent bond. In some embodiments the C-terminal and N-terminal recognition motif are comprised in the same protein, e.g., in the same amino acid sequence. Sortase-mediated conjugation of the substrates in such cases results in the formation of an intramolecular bond, e.g., a circularization of a single amino acid sequence, or, if multiple polypeptides of a protein complex are involved, the formation of an intra-complex bond. In some embodiments, the C-terminal and N-terminal recognition motifs are comprised in different amino acid sequences, for example, in separate proteins or other agents. Some sortase recognition motifs are described herein and additional suitable sortase recognition motifs are well known to those of skill in the art. For example, evolved sortases provided herein are evolved from a starting sortase (e.g. SEQ ID NO: 2) to recognize LMVGG [SEQ ID NO: 3]. In some embodiments, the starting sortase (e.g. SEQ ID NO: 2) recognizes LPESG [SEQ ID NO: 4]. As another example, sortase A of S. aureus recognizes and utilizes a C-terminal LPXT motif and an N-terminal GGG motif in transpeptidation reactions. In some embodiments, the LPXT motif comprises a C-terminal glycine (e.g., LPXTG; SEQ ID NO: 104). Additional sortase recognition motifs will be apparent to those of skill in the art, and the invention is not limited in this respect. A sortase substrate may comprise additional moieties or entities apart from the peptidic sortase recognition motif. For example, a sortase substrate may comprise an LPXTG [SEQ ID NO: 104] motif, the N-terminus of which is conjugated to any agent, e.g., a peptide or protein, a small molecule, a binding agent, a lipid, a carbohydrate, or a detectable label. Similarly, a sortase substrate may comprise a GGG motif, the C-terminus of which is conjugated to any agent, e.g., a peptide or protein, a small molecule, a binding agent, a lipid, a carbohydrate, or a detectable label. Accordingly, sortase substrates are not limited to proteins or peptides but include any moiety or entity conjugated to a sortase recognition motif.

The term “subject,” as used herein, refers to an individual organism, for example, an individual mammal. In some embodiments, the subject is a human. In some embodiments, the subject is a non-human mammal. In some embodiments, the subject is a non-human primate. In some embodiments, the subject is a rodent. In some embodiments, the subject is a sheep, a goat, a cow, a cat, or a dog. In some embodiments, the subject is a vertebrate, an amphibian, a reptile, a fish, an insect, a fly, or a nematode. In some embodiments, the subject is a research animal. In some embodiments, the subject is genetically engineered, e.g., a genetically engineered non-human subject. The subject may be of any sex and at any stage of development. In some embodiments, the subject suffers from chronic pain.

The term “target protein,” as used herein refers to a protein that comprises a sortase recognition motif. A target protein may be a wild-type protein, or may be an engineered protein, e.g., a recombinant protein.

### DETAILED DESCRIPTION

The extent and diversity of applications utilizing sortases as catalysts for transpeptidation reactions remain limited by the difficulty of finding in nature or creating in the laboratory highly active sortases that bind substrates having recognition motifs other than the canonical, or wild-type motif. One method for creating such sortases in the laboratory is through directed evolution.

Accordingly, some aspects of this invention provide novel evolved sortases generated by a directed evolution technology based on an integration of cell display (e.g., yeast display), enzyme-catalyzed small molecule-protein conjugation, and fluorescence-activated cell sorting (FACS), which provides a general strategy for the evolution of proteins that catalyze bond-forming reactions. In some embodiments, a sortase variant is produced using a yeast-display evolution technique, for example as described by Liu et al., U.S. Pat. No. 9,267,127, issued Feb. 23, 2016; Chen I, Dorr BM, and Liu DR., A general strategy for the evolution of bond-forming enzymes using yeast display. Proc Natl Acad Sci USA. 2011 Jul. 12; 108(28):11399, the entire contents of each are incorporated herein by reference. A yeast-display evolution technique was previously applied to evolve the bacterial transpeptidase sortase A of Staphylococcus aureus for improved catalytic activity, resulting in sortase variants with an improvement in activity and/or efficiency. See, e.g., Liu et al., U.S. Pat. No. 9,267,127, issued Feb. 23, 2016 and Liu et al., U.S. Pat. No. 10,202,593, issued Feb. 12, 2009. As provided herein, a yeast-display evolution technique was applied to produce evolved sortase variants capable of trans-peptidating AB proteins.

This disclosure provides data demonstrating the use of a yeast display selection and FACS strategy to generate an evolved a sortase (e.g., sortase A), over 16 rounds of evolution, that is capable of site-specifically modifying Alzheimer's disease-associated amyloid β-peptides (Aβ). As described in the examples, an evolved sortase (e.g, SrtAβ), that prefers LMVGG [SEQ ID NO: 3] substrates was derived from a starting sortase, eSrtA4S.6 (also referred to as “4S.6”), that prefers LPESG [SEQ ID NO: 4] substrates. In some embodiments, the evolved sortase prefers LMVGG [SEQ ID NO: 3] 30-fold over LPESG [SEQ ID NO: 4].

Other aspects of this invention relate to methods for producing the evolved sortases, and methods of using such sortases, for example, methods for detecting a target protein in a biological sample, as well as methods for inhibiting amyloid β-protein (Aβ) aggregation or plaque formation in a cell or subject, and methods for treating or ameliorating Alzheimer's disease (AD) in a subject.

### Evolved Sortases

This invention provides evolved sortases that recognize LMVGG [SEQ ID NO: 3] substrates. In some embodiments, an Alzheimer's disease-associated amyloid β-protein (Aβ) comprises the sequence LMVGG [SEQ ID NO: 3]. In some embodiments, an evolved sortase (e.g., SrtAβ) covalently modifies an amyloid β-protein (Aβ). In some embodiments, the modifying comprises conjugating a heterologous peptide to the amyloid β-protein (Aβ). In some embodiments, the protein is modified at the N-terminal, the C-terminal, or at both the N- and C-termini. In some embodiments, the protein is modified at the C-terminal.

In some embodiments, evolved sortases provided herein, bind LMVGG [SEQ ID NO: 3] substrates and are evolved from starting sortases that bind LPESG [SEQ ID NO: 4] substrates. In some embodiments, evolved sortases provided herein prefer LMVGG [SEQ ID NO: 3] substrates (e.g., have a higher binding affinity to LMVGG [SEQ ID NO: 3] relative to LPESG [SEQ ID NO: 4]). In some embodiments, starting sortases prefer LPESG [SEQ ID NO: 4] substrates (e.g., have a higher binding affinity to LPESG [SEQ ID NO: 4] relative to LMVGG [SEQ ID NO: 3]). In some embodiments, the starting sortase is derived from a wild-type S. aureus sortase A, which catalyzes a transpeptidation reaction that results in the conjugation of a peptide comprising a C-terminal sortase recognition motif with a peptide comprising an N-terminal sortase recognition motif. Naturally occurring sortases are typically selective for specific C-terminal and N-terminal recognition motifs, e.g., LPXTG [SEQ ID NO: 104] (where X represents any amino acid) and a triglyine (GGG), respectively. In some embodiments, the starting sortase (e.g., a sortase comprising the amino acid sequence set forth in SEQ ID NO: 2) binds substrates comprising the amino acid sequence LPESG [SEQ ID NO: 4]. In some embodiments, the starting sortase comprises a S. aureus sortase A sequence, or fragment thereof.

In some embodiments, the amino acid sequence of an evolved sortase provided herein comprises one or more substitutions (e.g., mutations) as compared to the sequence of the starting sortase (e.g., SEQ ID NO: 2), or a fragment thereof. For example, in some embodiments, the evolved sortase comprises at least three mutations, at least four mutations, at least five, at least six, at least seven, at least eight, at least nine, or at least 10 amino acid substitutions as compared to the amino acid sequence set forth in SEQ ID NO: 2 or a fragment thereof. In some embodiments, the evolved sortase sequence comprises 1, 2, 3, 4, 5, 6, 7, 8, 9, 10, 11, 12, 13, 14, 15, 16, 17, 18, 19, 20, 21, 22, 23, 24, 25, or more amino acid substitutions (e.g., mutations) as compared to the amino acid sequence set forth in SEQ ID NO: 2 or a fragment thereof. In some embodiments, the evolved sortase sequence provided comprises up to 50 amino acid substitutions as compared to the amino acid sequence set forth in SEQ ID NO: 2 or a fragment thereof. In some embodiments, the evolved sortase sequence provided comprises up to 40 amino acid substitutions as compared to the amino acid sequence set forth in SEQ ID NO: 2 or a fragment thereof.

In some embodiments, the sortase comprises at least 3, at least 4, at least 5, at least 6, at least 7, at least 8, at least 9, or at least 10 amino acid substitutions (e.g., mutations) as compared to the amino acid sequence set forth in SEQ ID NO: 2, or a fragment thereof, selected from the group consisting of the amino acid substitutions listed in Table 3. In some embodiments, the evolved sortase sequence comprises 1, 2, 3, 4, 5, 6, 7, 8, 9, 10, 11, 12, 13, 14, 15, 16, 17, 18, 19, 20, 21, 22, 23, 24, 25, or more amino acid substitutions (e.g., mutations) as compared to the starting sortase (e.g. SEQ ID NO: 2), selected from the group consisting of the amino acid substitutions listed in Table 3. In some embodiments, the evolved sortase sequence provided comprises up to 40 amino acid substitutions as compared to the amino acid sequence set forth in SEQ ID NO: 2, or a fragment thereof, selected from the group consisting of the amino acid substitutions listed in Table 3. The mutations disclosed herein are not exclusive of other mutations which may occur or be introduced. For example, a protease variant may have a mutation as described herein in addition to at least one mutation not described herein (e.g., 1, 2, 3, 4, 5, etc. additional mutations).

In some embodiments, the amount of variation between an evolved sortase and a starting sortase (e.g. SEQ ID NO: 2), or a fragment thereof is expressed as the percent identity at the amino acid sequence level. In some embodiments, the sequence of an evolved sortase is from about 70% to about 99.9% identical, 72% to about 98% identical, about 75% to about 95% identical, about 80% to about 90% identical, about 85% to about 95% identical, or about 95% to about 99% identical to the sequence set forth in SEQ ID NO: 2, or a fragment thereof, and comprises one or more amino acid substitutions selected from the group consisting of the amino acid substitutions listed in Table 3. In some embodiments, the sequence of an evolved sortase is at least 70% identical, at least 75% identical, at least 80% identical, at least 85% identical, at least 90% identical, at least 95% identical, at least 98% identical, at least 99% identical, or at least 99.5% identical to a starting sortase sequence (e.g. SEQ ID NO: 2), or a fragment thereof, and comprises one or more amino acid substitutions selected from the group consisting of the amino acid substitutions listed in Table 3. In some embodiments, the sequence of the evolved sortase is at least 80% identical to the sequence set forth in SEQ ID NO: 2, or a fragment thereof. In some embodiments, the evolved sortase is no more than 99.9% identical to the sequence set forth in SEQ ID NO: 2, or a fragment thereof.

In some embodiments, an evolved sortase binds substrates comprising the amino acid sequence LMVGG [SEQ ID NO: 3]. the evolved sortase comprises an amino sequence that is at least 80% identical (e.g., at least 80%, 85%, 90%, 95%, 98%, 99%, or 99.5% identical) to the amino acid sequence provided in SEQ ID NO: 2, or a fragment thereof, and the amino acid sequence of the sortase includes one or more substitutions (relative to SEQ ID NO: 2) selected from the group consisting of the amino acid substitutions listed in Table 3.

In some embodiments, an evolved sortase is provided comprising amino acid substitutions (relative to SEQ ID NO: 2) at two or more (e.g., 2, 3, 4, 5, 6, 7, 8, 9, 10, 11, 12, 13, 14, 15, 16, 17, 18, 19, 20, 21, 22, 23, 24, or 25) of the following positions: K62, A73, I76, R94, S102, E105, N107, S118, I123, D124, N127, G134, K138, G139, M141, K145, G147, N148, K152, M155, S157, R159, K162, D170, Q172, K173, K177, V182, V189, T196, R197, and K206.

In some embodiments, an evolved sortase is provided consisting of amino acid substitutions (relative to SEQ ID NO: 2) at two or more (e.g., 2, 3, 4, 5, 6, 7, 8, 9, 10, 11, 12, 13, 14, 15, 16, 17, 18, 19, 20, 21, 22, 23, 24, or 25) of the following positions: K62, A73, I76, R94, S102, E105, N107, S118, I123, D124, N127, G134, K138, G139, M141, K145, G147, N148, K152, M155, S157, R159, K162, D170, Q172, K173, K177, V182, V189, T196, R197, and K206.

In some embodiments, an evolved sortase is provided comprising amino acid substitutions (relative to SEQ ID NO: 2) at two or more (e.g., 2, 3, 4, 5, 6, 7, 8, 9, 10, 11, 12, 13, 14, 15, 16, 17, 18, 19, 20, 21, 22, 23, 24, or 25) of the following positions I76, S102, E105, N107, S118, I123, D124, N127, G134, K138, G139, M141, K145, K152, M155, R159, K162, Q172, K173, K177, V182, V189, T196, R197, and K206.

In some embodiments, an evolved sortase is provided consisting of amino acid substitutions (relative to SEQ ID NO: 2) at two or more (e.g., 2, 3, 4, 5, 6, 7, 8, 9, 10, 11, 12, 13, 14, 15, 16, 17, 18, 19, 20, 21, 22, 23, 24, or 25) of the following positions I76, S102, E105, N107, S118, I123, D124, N127, G134, K138, G139, M141, K145, K152, M155, R159, K162, Q172, K173, K177, V182, V189, T196, R197, and K206.

In some embodiments, the evolved sortase comprises or consists of two or more (e.g., 2, 3, 4) of the following substitutions relative to SEQ ID NO: 2: R94P, S118I, G134R, and V189F. In some embodiments, the sortase comprises or consists of the following amino acid substitutions relative to SEQ ID NO: 2: R94P, S118I, G134R, and V189F.

In some embodiments, the evolved sortase comprises or consists of two or more (e.g., 2, 3, 4, 5, 6) of the following substitutions relative to SEQ ID NO: 2: R94Y, S118I, A122W, D124G, G134R, and V189F. In some embodiments, the sortase comprises or consists of the following amino acid substitutions relative to SEQ ID NO: 2: R94Y, S118I, A122W, D124G, G134R, and V189F.

In some embodiments, the evolved sortase comprises or consists of two or more (e.g., 2, 3, 4, 5, 6, 7, 8, 9) of the following substitutions relative to SEQ ID NO: 2: R94Y, S118I, D124L, G134R, K138I, V182A, V189F, T196S, and R197S. In some embodiments, the sortase comprises or consists of the following amino acid substitutions relative to SEQ ID NO: 2: R94Y, S118I, D124L, G134R, K138I, V182A, V189F, T196S, and R197S.

In some embodiments, the evolved sortase comprises or consists of two or more (e.g., 2, 3, 4, 5, 6, 7, 8, 9, 10) of the following substitutions relative to SEQ ID NO: 2: R94Y, S118I, I123L, D124L, G134R, K138I, V182A, V189F, T196S, and R197S. In some embodiments, the sortase comprises or consists of the following amino acid substitutions relative to SEQ ID NO: 2: R94Y, S118I, I123L, D124L, G134R, K138I, V182A, V189F, T196S, and R197S.

In some embodiments, the evolved sortase comprises or consists of two or more (e.g., 2, 3, 4, 5, 6, 7, 8, 9, 10, 11) of the following substitutions relative to SEQ ID NO: 2: R94Y, S118I, I123L, D124L, G134R, K138I, K173E, V182A, V189F, T196S, and R197S. In some embodiments, the sortase comprises or consists of the following amino acid substitutions relative to SEQ ID NO: 2: R94Y, S118I, I123L, D124L, G134R, K138I, K173E, V182A, V189F, T196S, and R197S.

In some embodiments, the evolved sortase comprises or consists of two or more (e.g., 2, 3, 4, 5, 6, 7, 8, 9, 10, 11, 12, 13, 14) of the following substitutions relative to SEQ ID NO: 2: K62R, I76L, S118I, I123L, D124L, N127Y, G134R, K138L, K145T, R159C, V182A, V189F, T196S, and R197S. In some embodiments, the sortase comprises or consists of the following amino acid substitutions relative to SEQ ID NO: 2: K62R, I76L, S118I, I123L, D124L, N127Y, G134R, K138L, K145T, R159C, V182A, V189F, T196S, and R197S.

In some embodiments, the evolved sortase comprises or consists of two or more (e.g., 2, 3, 4, 5, 6, 7, 8, 9, 10, 11, 12, 13) of the following substitutions relative to SEQ ID NO: 2: K62R, N107D, S118I, I123L, D124L, G134R, K138I, K173E, K177R, V182A, V189I, T196S, and R197S. In some embodiments, the sortase comprises or consists of the following amino acid substitutions relative to SEQ ID NO: 2: K62R, N107D, S118I, I123L, D124L, G134R, K138I, K173E, K177R, V182A, V189I, T196S, and R197S.

In some embodiments, the evolved sortase comprises or consists of two or more (e.g., 2, 3, 4, 5, 6, 7, 8, 9, 10, 11, 12, 13, 14, 15, 16) of the following substitutions relative to SEQ ID NO: 2: K62R, I76L, N107D, S118I, I123L, D124L, G134R, K138I, S157R, R159H, K173E, K177R, V182A, V189I, T196S, and R197S. In some embodiments, the sortase comprises or consists of the following amino acid substitutions relative to SEQ ID NO: 2: K62R, I76L, N107D, S118I, I123L, D124L, G134R, K138I, S157R, R159H, K173E, K177R, V182A, V189I, T196S, and R197S.

In some embodiments, the evolved sortase comprises or consists of two or more (e.g., 2, 3, 4, 5, 6, 7, 8, 9, 10, 11, 12, 13, 14, 15, 16, 17, 18, 19) of the following substitutions relative to SEQ ID NO: 2: K62R, A73V, I76L, N107D, S118I, I123L, D124L, N127Y, G134R, K138L, K145T, R159C, Q172H, K173E, V182A, V189F, T196S, R197S, and K206E. In some embodiments, the sortase comprises or consists of the following amino acid substitutions relative to SEQ ID NO: 2: K62R, A73V, I76L, N107D, S118I, I123L, D124L, N127Y, G134R, K138L, K145T, R159C, Q172H, K173E, V182A, V189F, T196S, R197S, and K206E.

In some embodiments, the evolved sortase comprises or consists of two or more (e.g., 2, 3, 4, 5, 6, 7, 8, 9, 10, 11, 12, 13, 14, 15, 16, 17, 18, 19, 20) of the following substitutions relative to SEQ ID NO: 2: K62R, I76L, N107D, S118I, I123L, D124L, N127H, G134R, K138L, K145T, M155I, R159C, D170E, Q172H, K173E, V182A, V189F, T196S, R197S, and K206E. In some embodiments, the sortase comprises or consists of the following amino acid substitutions relative to SEQ ID NO: 2: K62R, I76L, N107D, S118I, I123L, D124L, N127H, G134R, K138L, K145T, M155I, R159C, D170E, Q172H, K173E, V182A, V189F, T196S, R197S, and K206E.

In some embodiments, the evolved sortase comprises or consists of two or more (e.g., 2, 3, 4, 5, 6, 7, 8, 9, 10, 11, 12, 13, 14, 15, 16, 17, 18, 19, 20, 21) of the following substitutions relative to SEQ ID NO: 2: K62R, A73V, I76L, N107D, S118I, I123L, D124L, N127H, G134R, K138L, K145T, N148S, M155I, R159C, D170E, Q172H, K173E, V182A, V189F, T196S, and R197S. In some embodiments, the sortase comprises or consists of the following amino acid substitutions relative to SEQ ID NO: 2: K62R, A73V, I76L, N107D, S118I, I123L, D124L, N127H, G134R, K138L, K145T, N148S, M155I, R159C, D170E, Q172H, K173E, V182A, V189F, T196S, and R197S.

In some embodiments, the evolved sortase comprises or consists of two or more (e.g., 2, 3, 4, 5, 6, 7, 8, 9, 10, 11, 12, 13, 14, 15, 16, 17, 18, 19, 20, 21) of the following substitutions relative to SEQ ID NO: 2: K62R, I76L, E105D, N107D, S118I, I123L, D124L, N127H, G134R, K138L, K145T, G147C, M155I, R159C, D170E, Q172H, K173E, V182A, V189F, T196S, and R197S. In some embodiments, the sortase comprises or consists of the following amino acid substitutions relative to SEQ ID NO: 2: K62R, I76L, E105D, N107D, S118I, I123L, D124L, N127H, G134R, K138L, K145T, G147C, M155I, R159C, D170E, Q172H, K173E, V182A, V189F, T196S, and R197S.

In some embodiments, the evolved sortase comprises or consists of two or more (e.g., 2, 3, 4, 5, 6, 7, 8, 9, 10, 11, 12, 13, 14, 15, 16, 17, 18, 19, 20, 21, 22, 23, 24, 25) of the following substitutions relative to SEQ ID NO: 2: I76L, S102C, E105D, N107D, S118I, I123L, D124L, N127H, G134R, K138L, G139D, M141I, K145T, K152R, M155I, R159C, K162R, Q172H, K173E, K177R, V182A, V189Y, T196S, R197S, and K206R. In some embodiments, the sortase comprises or consists of the following amino acid substitutions relative to SEQ ID NO: 2: I76L, S102C, E105D, N107D, S118I, I123L, D124L, N127H, G134R, K138L, G139D, M141I, K145T, K152R, M155I, R159C, K162R, Q172H, K173E, K177R, V182A, V189Y, T196S, R197S, and K206R. In some embodiments, the sortase comprises or consists of an amino acid sequence set forth in SEQ ID NO: 8.

In some embodiments, the evolved sortase comprises or consists of two or more (e.g., 2, 3, 4, 5, 6) of the following substitutions relative to SEQ ID NO: 2: S118I, G134R, R159C, K177R, V182A, and R197S. In some embodiments, the sortase comprises or consists of the following amino acid substitutions relative to SEQ ID NO: 2: S118I, G134R, R159C, K177R, V182A, and R197S.

In some embodiments, the evolved sortases provided herein bind substrates comprising the sequence LMVGG [SEQ ID NO: 3].

In some embodiments, the evolved sortase comprises any of the following sets of amino acid mutations listed below, relative to SEQ ID NO: 2:
R94P, S118I, G134R, and V189F
R94Y, S118I, A122W, D124G, G134R, and V189F
R94Y, S118I, D124L, G134R, K138I, V182A, V189F, T196S, and R197S
R94Y, S118I, I123L, D124L, G134R, K138I, V182A, V189F, T196S, and R197S
R94Y, S118I, I123L, D124L, G134R, K138I, K173E, V182A, V189F, T196S, and R197S
K62R, I76L, S118I, I123L, D124L, N127Y, G134R, K138L, K145T, R159C, V182A, V189F, T196S, and R197S
K62R, N107D, S118I, I123L, D124L, G134R, K138I, K173E, K177R, V182A, V189I, T196S, and R197S
K62R, I76L, N107D, S118I, I123L, D124L, G134R, K138I, S157R, R159H, K173E, K177R, V182A, V189I, T196S, and R197S
K62R, A73V, I76L, N107D, S118I, I123L, D124L, N127Y, G134R, K138L, K145T, R159C, Q172H, K173E, V182A, V189F, T196S, R197S, and K206E
K62R, I76L, N107D, S118I, I123L, D124L, N127H, G134R, K138L, K145T, M155I, R159C, D170E, Q172H, K173E, V182A, V189F, T196S, R197S, and K206E
K62R, A73V, I76L, N107D, S118I, I123L, D124L, N127H, G134R, K138L, K145T, N148S, M155I, R159C, D170E, Q172H, K173E, V182A, V189F, T196S, and R197S
K62R, I76L, E105D, N107D, S118I, I123L, D124L, N127H, G134R, K138L, K145T, G147C, M155I, R159C, D170E, Q172H, K173E, V182A, V189F, T196S, and R197S
I76L, S102C, E105D, N107D, S118I, I123L, D124L, N127H, G134R, K138L, G139D, M141I, K145T, K152R, M155I, R159C, K162R, Q172H, K173E, K177R, V182A, V189Y, T196S, R197S, and K206R
S118I, G134R, R159C, K177R, V182A, and R197S

In some embodiments, evolved sortases comprising the foregoing sets of amino acid mutations are those that bind substrates comprising LMVGG [SEQ ID NO: 3].

In some embodiments, the evolved sortase comprises any of the sequences listed herein including those found in any figures or any tables found in the application. In some embodiments, the evolved sortase comprises or consists of an amino acid sequence set forth in SEQ ID NO: 8.

In some embodiments, the evolved sortase utilizes a substrate different from those used by the starting sortase, e.g., LMVGG [SEQ ID NO: 3] substrate. In some embodiments, the evolved sortase binds substrates comprising the amino acid sequence LMVGG [SEQ ID NO: 3]. In certain embodiments, the evolved sortase has greater affinity for a particular recognition motif over another recognition motif (e.g. LMVGG [SEQ ID NO: 3]), but the motif may also be recognized albeit less well by the starting sortase. Therefore, the specificity of the evolved sortase has been altered as compared to the starting sortase.

In some embodiments, the specificity for a particular recognition motif is based on a comparison between the Kₘ that the evolved sortase has for the motif, relative to that of the starting sortase. For example, in some embodiments, an evolved sortase has a Kₘ for an altered recognition motif that is at least 2-fold, at least 3-fold, at least 4-fold, at least 5-fold, at least 6-fold, at least 7-fold, at least 8-fold, at least 9-fold, at least 10-fold, at least 20-fold, at least 30-fold, at least 40-fold, at least 50-fold, at least 75-fold, at least 100-fold, at least 125-fold, at least 150-fold, at least 200-fold, at least 250-fold, at least 300-fold, at least 400-fold, at least 600-fold, at least 800-fold, at least 1,000-fold, at least 1,200-fold, at least 1,400-fold, at least 1,600-fold, at least 1,800-fold, or at least 2,000-fold (or more) less than the Kₘ that the starting sortase exhibits for the altered recognition motif.

In some embodiments, an evolved sortase has increased activity on a substrate compared to activity of a starting sortase on the same substrate. In some embodiments, activity is measured by the ratio of cell surface biotinylation to sortase expression level. In some embodiments, an evolved sortase has increased activity (e.g., 2-fold, 5-fold, 10-fold, 50-fold, 100-fold, etc.) on substrates preferred by the evolved sortase, compared to the starting sortase (e.g. SEQ ID NO: 2). In some embodiments, an evolved sortase has increased activity of about 2-fold to about 30-fold, about 10-fold to about 100-fold, about 50-fold to about 500-fold, or about 100-fold to about 1000-fold, about 500-fold to about 5000-fold, or about 750-fold to about 10000-fold on a LMVGG [SEQ ID NO: 3] substrate compared to the starting sortase set forth in SEQ ID NO: 2.

In some embodiments, an evolved sortase has reduced activity on a substrate compared to activity of a starting sortase on the same substrate. In some embodiments, activity is measured by the ratio of cell surface biotinylation to sortase expression level. In some embodiments, an evolved sortase has reduced activity (e.g., 2-fold, 5-fold, 10-fold, 50-fold, 100-fold, etc.) on substrates preferred by the starting sortase, compared to the starting sortase (e.g. SEQ ID NO: 2). In some embodiments, an evolved sortase has reduced activity of about 2-fold to about 30-fold, about 10-fold to about 100-fold, about 20-fold to about 250-fold, about 50-fold to about 500-fold, or about 100-fold to about 1000-fold, about 500-fold to about 5000-fold, or about 750-fold to about 10000-fold on a LPESG [SEQ ID NO: 4] substrate compared to the starting sortase set forth in SEQ ID NO: 2.

In some embodiments, the starting sortase (e.g., SEQ ID NO: 2) is capable of modifying fetuin A in human plasma. In some embodiments, the evolved sortase has reduced activity (e.g., 2-fold, 5-fold, 10-fold, 50-fold, 100-fold, etc.) for a substrate sequence of fetuin A, compared to the starting sortase (e.g. SEQ ID NO: 2). In some embodiments, the native substrate sequence of fetuin is LPPAG [SEQ ID NO: 5]. In some embodiments, an evolved sortase has reduced activity of about 2-fold to about 30-fold, about 10-fold to about 100-fold, about 20-fold to about 250-fold, about 50-fold to about 500-fold, about 100-fold to about 1000-fold, about 500-fold to about 5000-fold, or about 750-fold to about 10000-fold on a LPPAG [SEQ ID NO: 5] substrate compared to the starting sortase set forth in SEQ ID NO: 2.

In some embodiments, an evolved sortase has a preference (e.g., 2-fold, 5-fold, 10-fold, 50-fold, 100-fold, etc.) for LMVGG [SEQ ID NO: 3] over LPESG [SEQ ID NO: 4] (e.g. greater binding affinity to LMVGG [SEQ ID NO: 3] relative to LPESG [SEQ ID NO: 4]. In some embodiments, the evolved sortase has a preference of about 2-fold to about 30-fold, about 10-fold to about 100-fold, about 20-fold to about 250-fold, about 50-fold to about 500-fold, or about 100-fold to about 1000-fold, about 500-fold to about 5000-fold, or about 750-fold to about 10000-fold for LMVGG [SEQ ID NO: 3] over LPESG [SEQ ID NO: 4]. The preference of the evolved sortases can be determined using various methods in the art. For example, in certain embodiments, the fold preference can be determined using flow cytometry assays and determining kinetic parameters as further described in the Examples below.

In some embodiments, an evolved sortase has a change in substrate preference of at least 5-fold, at least 6-fold, at least 7-fold, at least 8-fold, at least 9-fold, at least 10-fold, at least 20-fold, at least 30-fold, at least 40-fold, at least 50-fold, at least 75-fold, at least 100-fold, at least 125-fold, at least 150-fold, at least 200-fold, at least 250-fold, at least 300-fold, at least 400-fold, at least 600-fold, at least 800-fold, at least 1,000-fold, at least 1,200-fold, at least 1,400-fold, at least 1,600-fold, at least 1,800-fold, at least 2,000-fold (or more) to favor LMVGG [SEQ ID NO: 3] over LPESG [SEQ ID NO: 4]. In some embodiments, an evolved sortase has a change in substrate preference of at least 1,400-fold to favor LMVGG [SEQ ID NO: 3] over LPESG [SEQ ID NO: 4]. The change in substrate preference of the evolved sortases can be determined using various methods in the art. For example, in certain embodiments, the change in substrate preference can be determined using flow cytometry assays and determining kinetic parameters as further described in the Examples below.

In some embodiments, an evolved sortase provided herein is active in a buffer solution. In some embodiments, the buffer solution buffers at physiological pH, e.g., pH ranging from about 7.4 to about 7.6. In some embodiments, an evolved sortase provided herein is active in biological fluids. In some embodiments the biological fluid is human plasma. In some embodiments, the pH of blood plasma ranges from about 7.35 to about 7.45.

### Methods for Evolution of the Sortase Variants

Some aspects of this disclosure provide methods of producing the evolved sortase protein variants provided herein. In some embodiments, the methods comprise a step of (a) expressing in a population of yeast cells one or more fusion proteins, each fusion protein comprising a sortase protein (e.g. a S. aureus sortase A protein) or portion thereof conjugated to a triglycine (GGG) peptide having an N-terminus capable of reacting in sortase-catalyzed reactions. In some embodiments, the population of yeast displays a library of ˜10⁷ fusion proteins or greater. In some embodiments, the sortase protein is a starting sortase as described herein, and was previously evolved to recognize LPESG [SEQ ID NO: 4] substrates. In some embodiments, the sortase protein comprises the amino acid sequence set forth in SEQ ID NO: 2.

In some embodiments, the methods further comprise a step of (b) incubating the yeast cell population of (a) with a mixture comprising N-terminally biotinylated target substrates and non-biotinylated off-target substrates under conditions under which the sortases expressed by the yeast catalyze transpeptidation of the biotinylated target substrates to the surface of the yeast cells. Transpeptidation is the process of transferring an amino acid or group of amino acids from one compound to another (e.g. between triglycine and the target substrates). In some embodiments, a target substrate comprises the amino acid sequence LMVGG [SEQ ID NO: 3]. In some embodiments, an off-target substrate comprises the amino acid sequence LXXXG, where X represents any amino acid. An off-target substrate is a negative selection substrate that lack any biotinylated target substrate. In some embodiments, the off-target substrate comprises an amino acid sequence consisting of the group selected from LPESG [SEQ ID NO: 4], LMVTG [SEQ ID NO: 11], LPVGG [SEQ ID NO: 12], LAVGG [SEQ ID NO: 13], and LPPAG [SEQ ID NO: 5]. In some embodiments, the off-target substrate comprises the amino acid sequence LPESG [SEQ ID NO: 4]. In some embodiments, the off-target substrate comprises the amino acid sequence LMVTG [SEQ ID NO: 11]. In some embodiments, the off-target substrate comprises the amino acid sequence LPVGG [SEQ ID NO: 12]. In some embodiments, the off-target substrate comprises the amino acid sequence LAVGG [SEQ ID NO: 13]. In some embodiments, the off-target substrate comprises the amino acid sequence LPPAG [SEQ ID NO: 5].

Once the sortases catalyze transpeptidation, the sortases may be removed from the cell surfaces using a Tobacco Etch Virus (TEV) protease. For example, in some embodiments, the methods further comprise a step of (c) treating the yeast cells with TEV protease.

After removal of cell surface-displayed sortases with TEV protease, cells may be fluorescently-labeled to indicate active sortase variants. For example, in some embodiments, the method further comprises the step of (d) incubating the cells with fluorescently-labeled streptavidin under conditions under which the streptavidin binds to the biotin on the surface of the yeast cells comprising the target substrate. In some embodiments, the incubating occurs in human plasma.

Active sortase variants have higher on-target transpeptidation per unit expression than inactive variants or promiscuous variants and are isolated. For example, in some embodiments, the method further comprises the step of (e) isolating the fluorescently-labeled yeast cells form the population of yeast cells using fluorescence-activated cell sorting (FACS).

In some embodiments, step (c) occurs after steps (a) and (b). In some embodiments, step (e) occurs after steps (a) through (d). In some embodiments, the steps occur sequentially.

In some embodiments, steps (a) through (d) are repeated over multiple rounds. In some embodiments, the methods provided herein require many rounds (e.g., 2, 3, 4, 5, 6, 7, 8, 9, 10 or more generations) of evolution. In some embodiments, the methods provided herein require 16 rounds of evolution.

### Methods of Use/Treatment

Some aspects of this invention provide methods for detecting a target protein in a biological sample using the evolved sortases described herein. In some embodiments, the evolved sortase comprises or consists of an amino acid sequence set forth in SEQ ID NO: 8. In some embodiments, such methods include (a) contacting a biological sample (e.g., cerebrospinal fluid sample) with an evolved sortase provided herein and a probe comprising (i) one or more detectable agents and (ii) a peptide comprising the amino acid sequence GGGK [SEQ ID NO: 6]. In some embodiments, the contacting occurs under conditions under which the sortase conjugates the one or more detectable agents to the target protein. In some embodiments, the method further comprises (b) removing unconjugated probe from the biological sample and (c) detecting the presence of the detectable agent conjugated to the target protein. In some embodiments, the target protein is amyloid β-protein (Aβ). In some embodiments, the detectable agent comprises biotin. In some embodiments, the biotin comprises a fluorescent label. In some embodiments, the biological sample comprises cerebrospinal fluid (CSF). In some embodiments, the evolved sortase is an evolved S. aureus sortase A comprising two or more of the substitutions described herein. In some embodiments, the evolved sortase is an evolved S. aureus sortase A comprising consisting of or more of the substitutions described herein. In some embodiments, the evolved sortase is SrtAβ.

Some aspects of the disclosure provide methods for inhibiting amyloid β-protein (Aβ) aggregation or plaque formation in a cell or subject. In some embodiments, such methods comprise administering to the cell or subject an evolved sortase provided herein and a peptide comprising the amino acid sequence GGGRR [SEQ ID NO: 7]. In some embodiments, the GGGRR [SEQ ID NO: 7] is at the N-terminus of the peptide. In some embodiments, an evolved sortase covalently modifies an amyloid β-protein (Aβ). In some embodiments, the evolved sortase is an evolved S. aureus sortase A carrying two or more of the substitutions described herein, relative to SEQ ID NO: 2. In some embodiments, the evolved sortase is SrtAβ. In some embodiments, modification of amyloid β-protein (Aβ) comprises conjugating the peptide comprising the amino acid sequence GGGRR [SEQ ID NO: 7] to Aβ. In some embodiments, amyloid β-protein (Aβ) is modified at the C-terminus. In some embodiments, the last five residues of Aβ are replaced with GGGRR [SEQ ID NO: 7]. In some embodiments, the C-terminal modification of Aβ alters the aggregation propensity of the resulting peptides. In some embodiments, the cell is a mammalian cell. In some embodiments, the cell is a human cell. In some embodiments, the subject is mammal (e.g., a human or a non-human mammal). In some embodiments, the subject is a non-human mammal. In some embodiments, the subject is a human. In some embodiments, the cell is a central nervous system cell. In some embodiments, the cell is a neuron. In some embodiments, the subject has or is suspected of having Alzheimer's disease.

Some aspects of the disclosure provide methods for treating or ameliorating Alzheimer's disease (AD) in a subject. In some embodiments, such methods comprise administering to a subject having AD an evolved sortase provided herein and a peptide comprising the amino acid sequence GGGRR [SEQ ID NO: 7]. In some embodiments, the evolved sortase is an evolved S. aureus sortase A carrying two or more of the substitutions described herein. In some embodiments, the evolved sortase is SrtAβ. In some embodiments, the GGGRR [SEQ ID NO: 7] is at the N-terminus of the peptide. In some embodiments, the subject is mammal (e.g., a human or a non-human mammal). In some embodiments, the subject is a non-human mammal. In some embodiments, the subject is a human. In some embodiments, AD is associated with accumulated extracellular plaque deposits or aggregation of amyloid β protein (Aβ). In some embodiments, the methods for treating or ameliorating AD inhibit Aβ aggregation or plaque formation in a cell. In some embodiments, the cell is a central nervous system cell. In some embodiments, the cell is a neuron.

The function and advantage of these and other embodiments of the present invention will be more fully understood from the Examples below. The following Examples are intended to illustrate the benefits of the present invention and to describe particular embodiments, but are not intended to exemplify the full scope of the invention. Accordingly, it will be understood that the Examples are not meant to limit the scope of the invention.

### Example

Sortase transpeptidases are a superfamily of enzymes widely distributed throughout Gram-positive bacteria. Staphylococcus aureus sortase A (SrtA) is responsible for attaching proteins that contain a C-terminal LPXTG [SEQ ID NO: 104] sorting sequence to the cell wall. The enzyme cleaves between the threonine and glycine of the sorting sequence, forming an acyl-enzyme intermediate that subsequently acylates the primary amine of the pentaglycine of the peptidoglycan. SrtA shows a strong preference for its LPXTG [SEQ ID NO: 104] sorting sequence, but studies have revealed that it will accept a variety of glycine-based (and some non-glycine) nucleophiles. These properties make SrtA an attractive tool for site-specific protein modification. Indeed, SrtA has been successfully used for both C-terminal and N-terminal protein labeling, as well as protein circularization and the semi-synthesis of multi-domain proteins.

Yeast display and fluorescence-activated cell sorting (FACS) have been used to improve the kinetics of SrtA on LPETG [SEQ ID NO: 14], and to evolve sortase variants that accept single amino acid substitutions at the second or fourth position of the recognition sequence. This example describes reprogramming the specificity of SrtA to covalently modify the Alzheimer's disease-associated amyloid β-protein (Aβ). The formation of Aβ plaques in the central nervous system is the hallmark of Alzheimer's disease (AD). The ability to modify Aβ site-specifically might help illuminate its biological role, impede Aβ plaque formation, or facilitate understanding of AD pathogenesis. Since Aβ monomers are predominantly extracellular, unstructured, and contain a five-amino-acid sequence (LMVGG [SEQ ID NO: 3] at residues 34-38) that shares features with sortase's native recognition sequence, sortase-mediated conjugation is an attractive strategy to achieve site-specific modification of Aβ.

Over 16 rounds of evolution, a sortase variant, SrtAβ, that mediates the covalent modification of Aβ peptides was generated. SrtAβ was used to biotinylate and detect endogenous Aβ in clinical cerebrospinal fluid samples (CSF) at concentrations of 2-19 ng/mL. It was also demonstrated that SrtAβ-mediated conjugation of a hydrophilic pentapeptide to Aβ42 greatly slows the initiation of detectable aggregation. This work establishes the evolution of sortase enzymes to site-specifically modify naturally occurring proteins without requiring modification of endogenous genes.

### Materials and Methods

### Library Diversification

For diversification by error-prone, PCR, genes were isolated from harvested yeast libraries by PCR using the primers pCTCon2CTEV.HR2.Fwd (CCCATACGACGTTCCAGACTATGCAGGATCTGAGAACTTGTACTTTCAAGGTGCT [SEQ ID NO: 15]) and pCTCon2CTEV.HR2.Rev (CTGTTGTTATCAGATCTCGAGCTATTACAAGTCCTCTTCAGAAATAAGCTTTTGT TCGGA [SEQ ID NO: 16]), purified by gel electrophoresis, and subsequently mutagenized by using the GeneMorph II Random Mutagenesis Kit (Agilent) for 25 cycles of PCR amplification using primers pCTCon2CTEV.HR2.Fwd and pCTCon2CTEV.HR2.Rev. Reactions were purified by spin column and combined with NheI/BamHI-digested pCTCon2CTev vectors in a 5:1 insert:backbone mass ratio and electroporated into ICY200 as described below to yield yeast libraries.

For library diversification by site saturation mutagenesis (rounds 8 and 9), genes were isolated from harvested yeast libraries by PCR using the primers pCTCon2CTEV.HR2.Fwd and pCTCon2CTEV.HR2.Rev, purified by gel electrophoresis, and subcloned into pET29 via restriction digest with NheI/BamHI. This plasmid was used as the template for site-saturation mutagenesis with the following PNK-treated primers in Round 8:

182-NNK-Fwd:
[SEQ ID NO: 17]
NNKACCTGCGATGATTATAACTTTGAAACCG
182-NNK-Rev:
[SEQ ID NO: 18]
CAGGGTCAGCTGTTTATCTTTGCC
196-197-NNK-Fwd:
[SEQ ID NO: 19]
NNKNNKAAAATTTTTGTGGCGACCGAAGTG
196-197-NNK-Rev:
[SEQ ID NO: 20]
TTCCCACACGCCGGTTTC.

In Round 9, the following primers were used:

94-NNK-Fwd-1:
[SEQ ID NO: 21]
NNKGAACAGCTGGATCGTGGCGTGAGC
94-NNK-Fwd-2:
[SEQ ID NO: 22]
NNKGAACAGCTTGATCGTGGCGTGAGC
94-NNK-Rev:
[SEQ ID NO: 23]
GGTCGCCGGGCCCGG
122-124-NNK-Fwd-1:
[SEQ ID NO: 24]
NNKNNKNNKCGTCCGAACTATCAGTTTACCAACCTG
122-124-NNK-Fwd-2:
[SEQ ID NO: 25]
NNKNNKNNKCGTCCGTACTATCAGTTTACCAACCTG
122-124-NNK-Rev:
[SEQ ID NO: 26]
GGTATGGCCGATAATGCTAATGTTCTGATC.

Site-saturated genes were then amplified out of the pET29c backbone using primers pCTC-HR-pET29-Fwd (CCCATACGACGTTCCAGACTATGCAGGATCTGAGAACT TGTACTTTCAAGGTGCTAGCCAGGCGAGACCGCAGATTCC [SEQ ID NO: 27]) and pCTC-HR-pET29-Rev (CTGTTGTTATCAGATCTCGAGCTATTACAAGTCCTCTTC AGAAATAAGCTTTTGTTCGGA TCCTTTCACTTCGGTCGC [SEQ ID NO: 28]) and purified by gel electrophoresis.

For library diversification by DNA shuffling (round 15), the harvested library from the end of round 14 and the evolved sortase A pentamutant (5M) were each amplified with pCTCon2CTEV.HR2.Fwd (GTACTTTCAAGGTGCTAGCC [SEQ ID NO: 29]) and pCTCon2CTEV.HR2.Rev (CAGAAATAAGCTTTTGTTATC [SEQ ID NO: 30]) and purified by gel electrophoresis. 1 μg of each PCR product was added to 5 μL of 500 mM Tris-HCl pH 7.4, 100 mM MnCl2 and brought to 50 μL total volume. This mixture was incubated at 15° C. for 5 minutes at which point 0.5U of DNaseI was added. After 90 seconds, 1 μL of 500 mM EDTA was added to the reaction and the enzyme was heat killed at 90° C. for 10 minutes. The digest was run on a 3% agarose gel and 25-150 bp fragments were isolated. 200 ng of DNA fragments were added to a 100 μL primerless reassembly reaction with 5 μL 4 mM dNTPs, 4 μL 50 mM MgSO4, 10 μL 600 mM Tris-S04 (pH 8.9)/180 mM ammonium sulfate, 1U Taq polymerase, and 1U Phusion polymerase. This reaction was cycled at 94° C. for 2 min, then 35 cycles of (94° C. for 15 sec, 65° C. for 45 sec, 62° C. for 45 sec, 59° C. for 45 sec, 56° C. for 45 sec, 53° C. for 45 sec, 50° C. for 45 sec, 47° C. for 45 sec, 44° C. for 45 sec, 41° C. for sec, 68° C. for 45 sec), and then 68° C. 1 min. After PCR cleanup, a portion of the primerless reassembly product was amplified with primers CJP66-Fwd and CJP66-Rev, digested with NheI/BamHI and ligated into pCTCon2CTev vector.

### Yeast Library Construction

Fresh plates of ICY200 S. cerevisiae cells were streaked from long-term glycerol stocks and grown for 72 hours at 30° C. prior to use. A single colony was picked and grown in 10 mL YPD+100 U/mL penicillin, 100 μg/mL streptomycin, 100 μg/mL kanamycin overnight with shaking at 30° C. This suspension culture was freshly diluted into 125 mL YPD and electrocompetent cells. All library transformations were performed by gap repair homologous recombination into pCTCon2CTev vectors linearized by NheI and BamHI digestion. Following transformation, 10⁵ and 10⁶ dilutions were plated and used to estimate library size. Libraries were grown in SCD-Trp-Ura dropout media+100 U/mL penicillin, 100 μg/mL streptomycin, 100 μg/mL kanamycin at 30° C. Library expression was induced by transfer to SGR-Trp-Ura media at 20° C. overnight.

### GGGK[SEQ ID NO: 6]-CoA Synthesis

Fmoc-GGGK[SEQ ID NO: 6]-CONH₂ was dissolved in DMSO to a final concentration of 100 mM, then combined with 1.5 equivalents of LC-SMCC (Thermo-Fisher) and 2 equivalents of DIPEA (Sigma) in DMSO. The reaction was incubated for 1 hour at room temperature, then combined with 1.5 equivalents of coenzyme A trilithium hydrate (Sigma) in DMSO to a final peptide concentration of 25 mM and mixed at room temperature overnight. The Fmoc protecting group was removed with 20% vol/vol piperidine and incubation for 20 minutes. The reaction was quenched by the addition of 1 equivalent of TFA, and the product was purified on a preparative Kromasil 100-5-C18 column (21.2×250 mm, Peeke Scientific) by reverse phase HPLC (flow rate: 9.5 mL/min; gradient: 10% to 70% acetonitrile with 0.1% TFA in 0.1% aqueous TFA gradient over 30 minutes; retention time: 17.1 minutes). ESI-MS: [M-H]− m/z=1300.1 (observed); calculated for C45H72N14O23P3S−=1301.4. The concentration of GGGK[SEQ ID NO: 6]-CoA peptide was determined from the measured A259 using the known molar extinction coefficient of coenzyme A 53, 15,000 M⁻¹ cm⁻¹.

### Sfp Expression and Purification

E. coli BL21(DE3) harboring the pET29 expression plasmid for Sfp phosphopantetheinyl transferase were cultured at 37° C. in LB with 50 μg/mL kanamycin until OD600 ˜0.6. IPTG was added to a final concentration of 1 mM, and protein expression was induced at 37° C. for three hours. The cells were harvested by centrifugation and lysed by resuspension in B-PER(Novagen) containing 260 nM aprotinin, 1.2 μM leupeptin, 2 units/mL DNAseI, and 1 mM PMSF. The clarified supernatant was purified on Ni-NTA agarose, and fractions that were >95% pure were consolidated and dialyzed against 10 mM Tris pH 7.5+1 mM EDTA 5% glycerol. Enzyme concentration was calculated from the measured A280 using the published extinction coefficient of 27,220M⁻¹ cm⁻¹.

### TEV Protease Expression and Purification

E. coli BL21(DE3) harboring the pRK793 plasmid for TEV S219V expression and the pRIL plasmid (Addgene) was cultured in LB with 50 μg/mL carbenicillin and 30 μg/mL chloramphenicol until OD600 ˜0.7. IPTG was added to a final concentration of 1 mM, and the cells were induced for three hours at 30° C. The cells were pelleted by centrifugation and lysed by sonication. The clarified lysate was purified on Ni-NTA agarose, and fractions that were >95% TEV S219V were consolidated and dialyzed against TBS. Enzyme concentrations were calculated from A280 measurements using the reported extinction coefficient of 32,290 M⁻¹ cm⁻¹.

### Yeast Library Preparation and Fluorescence-Activated Cell Sorting

Induced cells were pelleted and resuspended in 10 mL TBS-B (100 mM Tris pH 7.5, 500 mM NaCl, 1% BSA). To this cell suspension was added 50 μL 1 M MgCl2, 10 μL 200 mM H₂NGGGK[SEQ ID NO: 6](CoA), and 50 μL 100 μM Sfp (10 mM Tris pH 7.5, 1 mM EDTA, 10% glycerol). The Sfp ligation reaction was incubated for 45 min at room temperature. Cells were then pelleted at 2400 g×10 min and the supernatant was removed. Desired sortase reaction buffer (TBS-BC; 100 mM Tris pH 7.5, 500 mM NaCl, 1% BSA, 5 mM CaCl₂), or PC; human plasma (GeneTex, GTX73265) centrifuged at 21000 g×10 min and passed through a 0.4 micron filter, 5 mM CaCl₂)) was then added and the cell pellet resuspended.

Separately, 100× target substrate and negative selection substrates were added to Eppendorf tubes. Typically, this involved 3-4 aliquots of varying substrate concentration such that a range of selection stringencies is represented across the aliquots. Cell suspension was added to the substrates, inverted to mix, and incubated for 15 to 60 min at room temperature. Cells were pelleted and treated with 1 mL TEV solution (100 μg/mL in PBS, 0.5% BSA, 2 mM EDTA) for 30 min on ice. Cells were pelleted and labeled with antibodies (1:200 Streptavidin-PE and 1:250 anti-HA Alexafluor-488, both from Invitrogen, in PBS, 0.5% BSA, 2 mM EDTA) for at least 30 min on ice. Cells were pelleted and washed once with 1 mL PBS, 0.5% BSA, 2 mM EDTA, then suspended in the same buffer before analysis and sorting on a BD FACS Aria Cell Sorter.

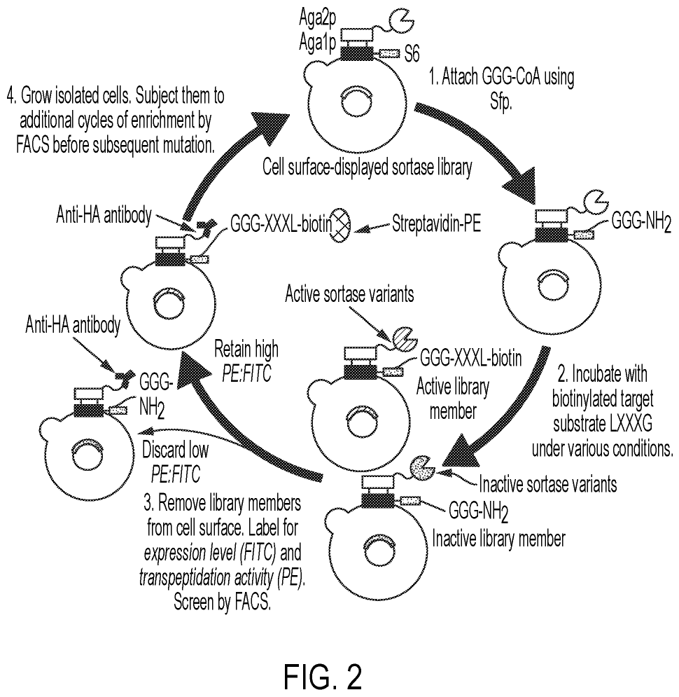

A negative control lacking any biotinylated target substrate was used to draw gates for sortase activity:expression level (PE:FITC) (see FIG. 2). Aliquots that contained target substrate were then analyzed, and the number of events in the PE:FITC gate was compared to the negative control. Aliquots that showed a >10-fold increase in gated events versus the negative control were considered suitable for sorting. The top 0.5-1.0% of cells were collected from a total number of events at least 10-fold greater than the estimated library size.

Cells sorted in active gate were collected in 2 mL SDC-Trp-Ura dropout media+100 U/mL penicillin, 100 μg/mL streptomycin, 100 μg/mL kanamycin in a 15 mL conical. Collected cells were then divided into 2-4 10 mL SDC-Trp-Ura cultures and grown at 30° C. for 2 days before they were induced again for a subsequent sort under more stringent conditions. Increased stringency was most commonly achieved by decreasing target substrate concentration, but occasionally by increasing off-target concentration or decreasing reaction times. Cycles of growth, induction, and enrichment were iterated until active variants could no longer be isolated using more stringent conditions than those used in the previous cycle, generally about 4-6 times. At this point, the surviving pool was extracted and re-diversified to create a library for the next round.

### Yeast Library Harvesting

Following the final FACS screen of a round, yeast were grown to saturation (OD ˜1.5) in SCD-Trp-Ura dropout media+100 U/mL penicillin, 100 μg/mL streptomycin, 100 g/mL kanamycin at 30° C., then lysed using a Zymo Research Zymoprep II kit according to manufacturer's instructions.

### Isolation of Single Clones

A portion of the harvested plasmid was transformed directly into Thermo Fisher One-Shot Machi T1 Chemically Competent cells according to manufacturer's instructions. 36-48 colonies, each bearing a single library member, were picked for rolling circle amplification and subject to Sanger sequencing with primers: CA205 (AGGCAATGCAAGGAGTTTTTG [SEQ ID NO: 32]) and CA232 (CAGTGGGAACAAAGTCGATTTTGTTACATCTAC [SEQ ID NO: 33]). Clones of interest were then subcloned into pET29 expression vectors. Alternatively, at the end of the last sort of a given round, the BD FACS Aria Cell Sorter was switched to plate mode, gates adjusted to only collect the top 0.1-0.3% of cells, and single cells collected in each well of a 96-well plate. After growing to saturation, these clones were subject to flow cytometry assays. Top performers were sequenced and then subcloned into pET29 expression vectors.

### Reversion Mutants

SrtAβ was subcloned into pET29 and used as PCR template for reactions with primers in Table 1. Following USER assembly or KLD ligation, products were transformed into Thermo Fisher One-Shot Machi T1 Chemically Competent cells. Following sequence verification, the reversion mutants were amplified out of the pET29 backbone with HR primers and transformed into ICY200 with NheI/BamHI-digested pCTCon2CTev vectors in a 5:1 insert:backbone mass ratio to yield yeast bearing single reversion mutants for flow cytometry analysis.

TABLE 1
Primers

| | | SEQ | | SEQ |
|---|---|---|---|---|
| | | ID | | ID |
| Mutant | Forward Primer | NO: | Reverse Primer | NO: |
| L761 | GGCGGGCTATATTGAAATTCC | 34 | ACTTTGCTTTTATCTTTCGG | 59 |
| C102S | ACCGTGGCG/ideoxyU/GTCCT | 35 | ACGCCACGG/ideoxyU/CGAGCTGTT | 60 |
| | TTGTG | | C | |
| D105E | AAGACGAAAGCC/ideoxyU/GG | 36 | AGGCTTTCGTCT/ideoxyU/CTTCCAC | 61 |
| | ATGATCA G | | AAAGCACACG CC | |
| D107N | GAAAGCCTGGATGA/ideoxyU/ | 37 | ATCATCCAGGCTT/ideoxyU/CGTTTT | 62 |
| | CAGAAC | | CGTCCACAAAG CAC | |
| I118S | GTCATACCGCGCT/ideoxyU/CT | 38 | AAGCGCGGTA/ideoxyU/GACCGGAA | 63 |
| | TCGTC | | ATGCTAATGTT CTGATCATCCAGG | |
| L1231 | ACTATCAGTT/ideoxyU/ACCAA | 39 | GTAAACTGATAG/ideoxyU/GCGGAC | 64 |
| | CCTGAG | | GAAGAATCGCG GTATGACCG | |
| L124D | TACCGCGCTTGACCGTCCGC | 40 | TGACCGATAATGCTAATGTTCTG | 65 |
| | ACT | | | |
| H127N | AACTATCAGTT/ideoxyU/ACCA | 41 | AAACTGATAGT/ideoxyU/CGGACGAA | 66 |
| | ACCTGAG G | | GAAGCGCG | |
| R134G | CGAAACTAGACAGCA/ideoxyU/ | 42 | GATGCTGTCTAGTT/ideoxyU/CGCCG | 67 |
| | CGTGT | | CCCCCAGGTT | |
| | | | GGTAAACTGATAGTGC | |
| L138K | GGCGGCGAAAAAAGACAGCA | 43 | CTCAGGTTGGTAAACTGATAG | 68 |
| | TCG | | | |
| D139G | AGCATCGTGTATTT/ideoxyU/A | 44 | GTAAAATACACGATGC/ideoxyU/GCC | 69 |
| | CAGTG | | TAGTTTCGCC GCCC | |
| I141M | ACTAGACAGCATGGTGTATTT | 45 | TTCGCCGCCCTCAGGTTG | 70 |
| | TACAGTG GG | | | |
| T145K | ATCGTGTATTTTAAAG/ideoxyU | 46 | ACTTTAAAATACACGA/ideoxyU/GCT | 71 |
| | /GGGCAA CGAAACCC | | GTCTAGTTTCG | |
| R152K | CGAAACCCGTAAGTATAAAAT | 47 | TTGCCCACTGTAAAATAC | 72 |
| | AACCAGC | | | |
| I155M | CCAGCATTTGTAACG/ideoxyU/ | 48 | ACGTTACAAATGC/ideoxyU/GGTCAT | 73 |
| | GAGAC | | TTTATATCTAC GGGTTTC | |
| C159R | AGCATTCGTAACG/ideoxyU/GA | 49 | ACGTTACGAATGC/ideoxyU/GGTTAT | 74 |
| | GACCGA CCG | | TTTATATCTAC GGG | |
| R162K | ACCGCGGTGGAAG/ideoxyU/G | 50 | CACTTCCACCGCGG/ideoxyU/CGGT | 75 |
| | CTGGAT G | | TTCACGTTACA AATGCTG | |
| H172Q | AGGAAGGCAAAGA/ideoxyU/A | 51 | ATCTTTGCCTTCC/ideoxyU/GTTCAT | 76 |
| | GACAGCT GAC | | CCAGCACTTCC AC | |
| E173K | ATAAAGGCAAAGA/ideoxyU/AG | 52 | ATCTTTGCCTTTA/ideoxyU/GTTCATC | 77 |
| | ACAGCT GAC | | CAGCACTTCC AC | |
| R177K | AGGCAAAGATAAACAGCTGAC | 53 | TCATGTTCATCCAGCACTTCC | 78 |
| | CC | | | |
| A182V | ACCTGCGATGAT/ideoxyU/ATA | 54 | AATCATCGCAGG/ideoxyU/GACCAG | 79 |
| | ACTATG | | GGTCAGCTGTC TATC | |
| Y189V | AAACCGGCGTG/ideoxyU/GGG | 55 | ACACGCCGGTT/ideoxyU/CTACGTTA | 80 |
| | AATCCAG | | TAATCATCGCA GGTCGC | |
| S196T | CGTGTGGGAAACTAGTAAAAT | 56 | CCGGTTTCATAGTTATAATC | 81 |
| | TTTTG | | | |
| S197R | GTGGGAATCCCGTAAAATTTT | 57 | ACGCCGGTTTCATAGTTATAATC | 82 |
| | TGTGG | | | |
| R206K | ACCGAAGTGAAAGGA/ideoxyU | 58 | ATCCTTTCACTTCGG/ideoxyU/CGCC | 83 |
| | /CCGAAC AAAAGCTTATTTC | | AC | |

### Minimal Mutant

SrtA 4S.6 was subcloned into pET29 and used as PCR template for two reactions, one with primers (ATCGTCCGAAC/ideoxyU/ATCAGTTTACCAACCTGCGCGCGGCGAAA AAAGGCAGC [SEQ ID NO: 84]) and (AGGGTCAGC/ideoxyU/GTCTATCTTTGCCTTTCTGTTCATCCAGCACTTCC [SEQ ID NO: 85]), the other with primers (AGCTGACCC/ideoxyU/GGCGACCTGCGATGATTAT AACGTGGAAACCG [SEQ ID NO: 86]) and (AGTTCGGACGA/ideoxyU/CAATCGCGGTATGGCCGATAATGCTAATGTTCTGATC ATCCAGGC [SEQ ID NO: 87]). USER assembly of these two fragments yielded 4S.6 with S118I, G134R, K177R, and V182A mutations. This mutant version of 4S.6 was used as template for two further PCRs, one with primers (ACCAGCATTTGTAACG/ideoxyU/GAAACCGACCGCGGTGG [SEQ ID NO: 88]) and (AAAAATTTTACTGGTT/ideoxyU/CCCACACGCCGGTTTCCAC [SEQ ID NO: 89]), the other with primers (AAACCAGTAAAATTTT/ideoxyU/G TGGCGACCGAAGTGAAAGGATCC [SEQ ID NO: 90]) and (ACGTTACAAATGCTGG/ideoxyU/CATTTTATATTTACGGGTTTCGTTGC [SEQ ID NO: 91]). USER assembly of these two fragments yielded the minimal mutant, 4S.6 with S118I, G134R, R159C, K177R, V182A, and R197S mutations. This mutant was then amplified out of the pET29 backbone with HR primers and transformed into ICY200 and ligated into NheI/BamHI-digested pCTCon2CTev by homologous recombination.

Yeast Transformation with LiAc/ss Carrier DNA/PEG

A 10 mL ICY200 starter culture in YPD (100 U/mL penicillin, 100 μg/mL streptomycin, and 50 μg/mL kanamycin) was grown overnight at 30° C. Cells were centrifuged at 2500 g×10 min, before removal of the supernatant and two washes with 25 mL water. Cells were resuspended in 1 mL water and transferred to a 1.5 mL Eppendorf tube. Cells were pelleted and washed once more before being resuspended in 1 mL of water and split into 100 μL aliquots. Aliquots were pelleted and supernatant removed. To each cell pellet was added 240 μL PEG 3550 (50% w/v), 36 μL LiOAc (1.0 M), 50 μL single stranded carrier DNA (2.0 mg/mL), 34 μL plasmid DNA or fragments (500-1000 ng) plus sterile water. Cells were then heat shocked at 42° C. for 40 min. Following heat shock, the cells were spun at 2500 g×10 min, supernatant was removed, and the pellet was resuspended in 1 mL water. 10-100 μL of cell suspension was plated on SDC-Trp-Ura dropout plates and grown at 30° C. for 2-3 days.

### Flow Cytometry Analysis

Single clones were assayed by flow cytometry in a process similar to a library being prepared for sorting. Once a single clone was obtained via single cell sorting or lithium acetate transformation, it was grown to saturation in SDC-Ura-Trp dropout media and then induced overnight in SGR. Triglycine was conjugated to the cell surface by Sfp as with a library, with the volume scaled down depending on culture size. Reactions of surface-displayed sortases, TEV cleavage, and labeling are carried out as with a library preparation before analysis on a Bio-Rad ZE5 Cell Analyzer.

### Sortase Expression and Purification

E. coli BL21(DE3) transformed with pET29 sortase expression plasmids were cultured at 37° C. in LB with 50 μg/mL kanamycin until OD600=0.5-0.8. IPTG was added to a final concentration of 1 mM and protein expression was induced overnight at 16° C. The cells were harvested by centrifugation and resuspended in lysis buffer (50 mM Tris pH 8.0, 300 mM NaCl supplemented with 1 mM MgCl2, 2 units/mL DNAseI (NEB), 260 nM aprotinin, 1.2 μM leupeptin, and 1 mM PMSF). Cells were lysed by sonication and the clarified supernatant was purified on Ni-NTA agarose following the manufacturer's instructions. Fractions that were >95% purity, as judged by SDS-PAGE, were consolidated and buffer exchanged into 25 mM Tris pH 7.5, 150 mM NaCl, 10% glycerol, 1 mM TCEP by size-exclusion chromatography in this buffer on a Superdex 200 Increase 10/300 GL column (GE). Enzyme concentrations were calculated by reducing agent-compatible BCA Protein Assay Kit (Pierce).

### Aβ-GGGRR[SEQ ID NO: 7] Cloning

Expression plasmid Aβ42/pET3 was amplified with primers GGRR[SEQ ID NO: 102]-Fwd (CGCCGTTAATAGGAGCTCGATCCGG [SEQ ID NO: 92]) and GGRR[SEQ ID NO: 102]-Rev (CCCACCGCCACCAACCATCA [SEQ ID NO: 93]). The PCR product was ligated with KLD enzyme mixture (New England BioLabs) and transformed into One-Shot Machi T1 Chemically Competent cells, from which Aβ37-GGGRR[SEQ ID NO: 7]/pET3 was sequence verified and isolated.

### Aβ Expression and Purification

E. coli BL21(DE3) transformed with pET3 Aβ expression plasmids (AβM1-40, AβM1-42, or AβM1-37GGGRR [SEQ ID NO: 7]) were cultured at 37° C. in LB-Carb until OD600=0.5-0.6. IPTG was added to a final concentration of 1 mM (AβM1-40 and AβM1-42) or 0.1 mM (AβM1-37GGGRR [SEQ ID NO: 7]) and protein expression was induced for 4 hours at 37° C. For AβM1-40 and AβM1-42, cells were pelleted and lysed by resuspension in 10 mM Tris-HCl pH 8.0, 1 mM EDTA and sonication. Following lysis, the lysate was centrifuged for 10 minutes at 18,000 g. Supernatant was discarded and pellet was resuspended in 10 mM Tris-HCl pH 8.0, 1 mM EDTA. Sonication, centrifugation, and removal of supernatant were repeated to yield an insoluble pellet. For AβM1-37GGGRR [SEQ ID NO: 7], cells were pelleted and lysed using B-PER bacterial protein extraction reagent (Thermo) supplemented with DNAseI and lysozyme and then centrifuged for 10 minutes at 18,000 g, with the insoluble pellet retained.

Insoluble pellets were resuspended in 8M urea, 10 mM Tris/HCl pH 8.0, 1 mM EDTA and then sonicated. Solubilized inclusion bodies were diluted with 10 mM Tris-HCl pH 8.0, 1 mM EDTA and added to pre-equilibrated DEAE-sepharose. After a 20-30 minute incubation, resin was batch filtered, washed for 5 minutes with 50 mM Tris pH 8.5, and then washed again for 5 minutes with 50 mM Tris pH 8.5, 25 mM NaCl. Following washes, recombinant peptides were eluted from resin with 50 mM Tris pH 8.5, 125 mM NaCl and lyophilized.

### Chemically Synthesized Aβ

Aβ1-40 and Aβ1-42 peptides (including Btn-LC-Aβ40 and Btn-LC-Aβ42) were synthesized and purified using reverse-phase HPLC. Peptide mass and purity (>99%) were confirmed by reverse-phase HPLC and electrospray ion trap mass spectrometry.

### Isolation of Aβ Monomers

Lyophilized Aβ peptides, whether synthetic in origin (ERI Amyloid Laboratory, LLC), or produced by recombinant technology, were dissolved in 7 M guanidium chloride, 50 mM Tris pH 7.5, 2 mM EDTA at a concentration of 1 mg/mL and incubated overnight. Denatured Aβ was then purified by size exclusion chromatography using a Superdex 75 300/10 column (GE) at a flow rate of 0.5 mL/min in alkaline buffer (50 mM Tris-HCl pH 8.5) to minimize peptide aggregation. Peptide concentration was measured by A275 (F=1361 M−1 cm−1). Peptide was either used immediately after purification or diluted to 20 μM, aliquoted, and frozen at −80° C. for later use.

### Western Blot for Fetuin A

Samples of sortase reactions with fetuin A were added to 4×NuPAGE lithium dodecyl sulfate (LDS) buffer (Invitrogen, NP0007), heat denatured, and loaded onto a 4-12% bis-tris gel and ran at 160 V for 30 min in MES running buffer. Samples from reactions in plasma were diluted as follows: 20 μL sample diluted+30 μL TBS+20 μL LDS buffer, for a total dilution of 3.5×. Gels were transferred to PVDF membrane via iBlot and membrane blocked with Superblock Blocking Buffer (Thermofisher, 37515) for 1 hour at room temperature. The membrane was then incubated with mouse anti-fetuin A antibody (Abcam, ab89227, 1:500 dilution in Superblock TBS+0.1% tween-20) overnight at 4° C. followed by washing in PBS-T (PBS+0.1% tween-20) three times for 5 minutes each. Secondary antibodies Streptavidin-IR800 (Licor, 926-32230) and goat anti-mouse-IR680LT (Licor, 926-68020) (both 1:10,000 dilution in Odyssey Block in PBS (Licor, 927-40000), 0.1% Tween-20, 0.01% SDS) were applied for 30 min at room temperature in the dark. The membrane was washed with PBS-T three times for 5 minutes each, followed by one wash with MilliQ water and imaged on an Odyssey Imager.

### Western Blot for Aβ

Samples of sortase reactions with Aβ were added to 4×LDS buffer and, without heat denaturing, loaded onto a 4-12% bis-tris gel and ran at 160 V for 30 min in MES running buffer. Gels were transferred to PVDF membrane via iBlot and membrane blocked with Superblock Blocking Buffer (Thermofisher, 37515) for 1 hour at room temperature. The membrane was then incubated with mouse anti-Aβ 4G8 antibody (Biolegend, 800702, 1:1000 dilution in Superblock TBS+0.1% tween-20) overnight at 4° C. followed by washing in PBS-T (PBS+0.1% tween-20) three times for 5 minutes each. Secondary antibodies Streptavidin-IR800 (Licor, 926-32230) and goat anti-mouse-IR680LT (Licor, 926-68020) (both 1:10,000 dilution in Odyssey Block in PBS (Licor, 927-40000), 0.1% Tween-20, 0.01% SDS) were applied for 30 min at room temperature in the dark. The membrane was washed with PBST three times for 5 minutes each, followed by one wash with MilliQ water and imaged on an Odyssey Imager.

### Streptavidin Pulldown of Sortase Labeled Plasma Proteins

1 mL of normal human plasma was combined with 10 μL of 0.1M GGK (Biotin) and 10 μL of 100 μM sortase 4S.6 or SrtAβ, then incubated at room temperature for 2 hours. 100 μL of pre-equilibrated Ni-NTA resin slurry was added to the mixture and incubated at room temperature with shaking for 15 minutes before being filtered through a 0.2 μm spin filter before dilution to 10 mL final volume in PBS-E (PBS+1 mM EDTA). The solution was concentrated using a 3 kDa molecular weight cut-off spin concentrator for 30 minutes at 3500×g and a final volume of <1 mL. The samples were diluted with PBS-E to 10 mL final volume, re-concentrated, and re-diluted in a total of six wash cycles to give an expected small molecule biotin concentration of <1 nM. The concentrated mixture was then incubated with 200 μL of pre-equilibrated Invitrogen MyOne Streptavidin Dynabeads with shaking for 30 minutes before magnetic separation and washing three times with PBS+0.1% Tween-20. Beads were then resuspended in 100 μL SDS-PAGE loading buffer with 100 μM free biotin and incubated at 95° C. for 15 minutes. A 15p L aliquot was then run on a 4-12% Bis-Tris PAGE gel and visualized by staining with Coomassie blue.

### HPLC Assay of Sortases on LMVGG [SEQ ID NO: 3]

Reactions were performed with Abz-LMVGGK[SEQ ID NO: 103](Dnp)-CONH₂ peptide fixed at 10 μM. Reaction conditions were 300 mM Tris pH 7.5, 150 mM NaCl, 100 mM H2N-GGG-COOH, 5 mM CaCl₂), 5% v/v DMSO. 5 μL of 10 μM sortase stock was added to 45 μL reaction buffer, yielding a final enzyme concentration of 1 μM. Reactions were incubated for 120 min at 22.5° C. Reactions were quenched with 10 μL 1 N HCl. The total volume of each reaction was transferred to HPLC sample vials and ran on analytical reverse phase Agilent Zorax SB-C18 (2.1×150 mm, 5 m) and chromatographed using a linear gradient 10 to 56.5% acetontrile with 0.1% TFA in 0.1% aqueous TFA over 13 minutes. To calculate the percent conversion, the ratio of the integrated areas of the GK(Dnp)-CONH₂ (rt=6.7 minutes) and Abz-LMVGG[SEQ ID NO: 3](Dnp)-CONH₂ (rt=11.6 minutes) Abs355 peaks were compared directly.

### HPLC Assay of Sortases on Aβ40

Reactions were performed with 20 μM Aβ40 and 1 mM GGGK[SEQ ID NO: 6](Dnp) in 50 mM Tris pH 8.5, 150 mM NaCl, and 5 mM CaCl₂). 5 μM of SrtAβ was added to this mixture and incubated at room temperature overnight. Reactions were quenched with 10 μL 1 N HCl. The total volume of each reaction was transferred to HPLC sample vials and ran on analytical reverse phase Agilent Zorax SB-C18 (2.1×150 mm, 5 m) and chromatographed using a linear gradient 10 to 56.5% acetontrile with 0.1% TFA in 0.1% aqueous TFA over 13 minutes. To calculate the percent conversion, the ratio of the integrated areas of the GGGK[SEQ ID NO: 6](Dnp) (rt=8.2 minutes) and Aβ37-GGGK[SEQ ID NO: 6](Dnp) (rt=12.6 minutes) Abs355 peaks were compared directly.

### Kinetic Assay of Sortases on LPESG [SEQ ID NO: 4]

Assays to determine k_{cat} and K_{m LPESG[SEQ ID NO: 4]} were performed in 300 mM Tris pH 7.5, 150 mM NaCl, 5 mM CaCl₂), 5% v/v DMSO, and 10 mM Gly-Gly-Gly-COOH (GGG). The concentration of the LPESG [SEQ ID NO: 4] peptide substrate ranged from 62.5 μM to 4 mM, and enzyme concentrations ranged from 100 nM to 1000 nM. Reactions were initiated with the addition of enzyme and incubated at 22.5° C. for 7 minutes (sortase 4S.6) or 2 hours (SrtAβ) before quenching with 0.2 volumes of 5 M HCl. 5 to 10 nmol of peptide from the quenched reactions were injected onto an analytical reverse-phase Eclipse XDB-C18 HPLC column (4.6×150 mm, 5 m, Agilent Technologies) and chromatographed using a linear gradient of 10 to 65% acetonitrile with 0.1% TFA in 0.1% aqueous TFA over 13 minutes. Retention times under these conditions for the Abz-LPESGK[SEQ ID NO: 31](Dnp)-CONH₂ substrate and the released GKDnp peptide were 12.8 and 10.4 minutes, respectively. To calculate the percent conversion, the ratio of the integrated areas of the GK(Dnp)-CONH₂ and Abz-LPESGK[SEQ ID NO: 31](Dnp)-CONH₂ peptide Abs355 peaks were compared directly. To determine k_{cat} and K_{m, LPESG[SEQ ID NO: 4]}, reaction rates were fit to the Michaelis-Menten equation in GraphPad Prism.

### Kinetic Assay of Sortases on LMVGG [SEQ ID NO: 3]

Assays to determine k_{cat} and K_{m LMVGG[SEQ ID NO: 3]} were performed in 300 mM Tris pH 7.5, 150 mM NaCl, 5 mM CaCl₂), 5% v/v DMSO, and 10 mM Gly-Gly-Gly-COOH (GGG). The concentration of the Abz-LMVGG[SEQ ID NO: 3](Dnp)-CONH₂ peptide substrate ranged from 10 to 200 μM with enzyme concentration of 1 μM. Reactions were conducted in 96-well half area black/clear flat bottom plates (Corning) and initiated with the addition of enzyme. Plates were incubated at 24° C. and monitored for increase in fluorescence (ex=317 nm, em=420 nm) in a Tecan plate reader for 2 hours. Changes in fluorescence were converted to molar velocities using calibration curves of Abz-LMVGG[SEQ ID NO: 3](Dnp)-CONH₂ and a 1:1 mixture of free Abz and Dnp. Inner filter quenching effects were corrected using Fcorr=Fobs×antilog[(Aex+Aem)/2], where Fcorr is the corrected fluorescence value, Fobs is the observed fluorescence value, Aex is the absorbance at 317 nm, and Aabs is the absorbance at 420 nm. To determine k_{cat} and K_{m,LMVGG[SEQ ID NO: 3]}, initial velocities were fit to the Michaelis-Menten equation in GraphPad Prism.

### Semi-Synthesis of Aβ(M1-37-GGGK[SEQ ID NO: 6](Btn))

Freshly purified Aβ(M1-40) monomers (120 μM in 50 mM Tris pH 8.5) were supplemented with 150 mM NaCl, 5 mM CaCl₂), and 1 mM TCEP and reacted overnight at room temperature with 50M SrtAβ and 1 mM GGGK[SEQ ID NO: 6](Btn). After desalting in a 3 kDa molecular weight cutoff spin filter, the reaction mixture was lyophilized and then dissolved in 7 M guanidium chloride, 50 mM Tris pH 7.5, 2 mM EDTA and ran on a Kinetex C18 100 Å (150×30 mm, 5 m, Phenomenex) column. The acetonitrile concentration was increased from 10 to 35% over the first 5 minutes, 35 to 38% over the next 6 minutes, and then from 38 to 90% over the next 5 minutes. The major peak eluted at 12.8 minutes. This was confirmed to be Aβ(M1-37-GGGK[SEQ ID NO: 6](Btn)) by LC/MS (m/z=4731.98 observed, 4730.27 expected) and the product was lyophilized and stored at −20° C. for later use.

### Streptavidin Capture ELISA for Detection of Biotinylated Aβ

Aβ(M1-37-GGGK[SEQ ID NO: 6](Btn)) standards were prepared in diluent (TBS+0.1% Tween-20+1% BSA) in a range of concentrations from 20 nM to 312 μM. Samples were diluted as necessary in this same diluent. Pre-blocked Streptavidin Coated High Capacity plates (clear, 96-well, Pierce) were washed 2× with TBS-T (TBS+0.1% Tween-20) before addition of standards and any samples. Biotinylated material was captured by streptavidin at room temperature for 2 hours. Plates were washed three times with TBS-T. 100 μL of mouse anti-Aβ clone 4G8 (1:2000 in diluent, Biolegend, 800702) was added to each well and incubated at room temperature for one hour. Following 3×TBS-T washes, each well was treated with 100 μL of goat anti-mouse IgG HRP conjugate (1:4000 in diluent, ThermoFisher, A-10668) for 30 minutes at room temperature. Plates were washed four times with TBS-T before addition of 50 μL TMB (ThermoFisher, 34028). Wells were allowed to develop until saturation and then quenched with 50 μL of 2M H₂SO₄. The absorbance of each well at 450 nm was then measured using a Tecan Plate Reader. The standard curve was fitted to 4-parameter logistics curve by Solver in Excel and used to calculate concentration of biotinylated Aβ in present samples.

### M266 ANTIBODY CAPTURE ELISA FOR DETECTION OF BIOTINYLATED Aβ

Thermo Nunc Maxisorp plates (96-well, clear) were incubated overnight with 100 μL of 3 μg/mL anti-Aβ antibody m266. The next day, plates were washed 3× with TBS-T and blocked for 2 hours with 5% MSD Blocker A (Meso Scale, Rockville, MD) in TBS-T. Aβ(M1-37-GGGK[SEQ ID NO: 6](Btn)) standards were prepared in diluent (1% MSD Blocker A in TBS-T) in a range of concentrations from 2.5 ng/mL to 39 pg/mL. Samples were diluted as necessary in this same diluent. Plates were washed 3× with TBS-T before addition of standards, samples, and blanks in triplicate. After 2 hours of capture, plates were washed 3× with TBS-T. 100 μL of streptavidin-HRP (1:100 in diluent, R&D Systems Part #890803) was added to each well for 30 minutes. After 4×TBS-T washes, wells were developed with 50 μL TMB (Thermo N301) and quenched with 2M H₂SO₄. The absorbance at 450 nm of each well was then measured by a Molecular Devices plate reader. The standard curve was fitted to 4-parameter logistics curve by Solver in Excel and used to calculate the concentration of biotinylated Aβ present in samples.

### ELISA Assays for Aβ40 and Aβ42

Thermo Nunc Maxisorp plates (96-well, clear) were incubated overnight with 100 μL of 3 μg/mL anti-Aβ antibody m266. The next day, plates were washed 3× with TBS-T and blocked for 2 hours with 5% MSD Blocker A in TBS-T. Aβ40 and Aβ42 standards were prepared in diluent (1% MSD Blocker A in TBS-T) in a range of concentrations from 2.5 ng/mL to 39 pg/mL. Samples were diluted as necessary in this same diluent. Plates were washed 3× with TBS-T before addition of standards, samples, and blanks in triplicate. After a 2-hour capture, plates were washed and secondary antibodies (1:2500 biotinylated 21F12 for Aβ42, 1:4000 biotinylated 2G3 for Aβ40) were added for 2 hours. After another set of washes, streptavidin-HRP (1:100) was added for 30 minutes. Plates were then washed, developed with TMB, and quenched with H₂SO₄. The absorbance at 450 nm of each well was measured by a Molecular Devices plate reader. The standard curves were fitted to 4-parameter logistics curve by Solver in Excel and used to calculate concentrations of Aβ40 and Aβ42 present in the samples.

CSF Labeling with GGG Using SrtAβ

Aliquots of CSF collected from 10 different patients were supplemented with 5 mM CaCl₂) and treated for 1 hour with 5 μM SrtAβ and 500 μM GGG. Reactions were quenched with addition of 5 mM EDTA and diluted 2-fold with 1% MSD Blocker A in TBS-T. Part of the sample was set aside for Aβ42 measurement, while the rest was diluted 5-fold (total dilution=10-fold) for Aβ40 measurement. Untreated aliquots from the same patients were diluted similarly. Aβ40 and Aβ42 were captured by anti-Aβ antibody m266 and detected with C-terminal specific antibodies as described above.

CSF Labeling with GGGK[SEQ ID NO: 6](Btn) Using SrtAβ

Aliquots of CSF collected from 10 different patients were supplemented with 5 mM CaCl₂) and treated for 2 hours with 5 μM SrtAβ and 500 μM GGGK[SEQ ID NO: 6](Btn). Reactions were quenched with addition of 5 mM EDTA and all samples (full reactions, no SrtAβ control, no GGGK[SEQ ID NO: 6](Btn) control, and untreated) were diluted 10-fold with 1% MSD Blocker A in TBS-T. Biotinylated Aβ was captured by anti-Aβ antibody m266 and detected without secondary antibody using streptavidin-HRP as described above.

### Semi-Synthesis of Aβ(M1-37-GGGRR [SEQ ID NO: 7])

Immediately following elution from DEAE resin in 50 mM Tris pH 8.5+125 mM NaCl, recombinant Aβ42 (20 mL of estimated concentration 40 μM=3-4 mg) was supplemented with 5 mM CaCl₂) and 5 mM DTT and treated overnight at room temperature with 20 μM SrtAβ and 200 μM GGGRR [SEQ ID NO: 7]. The reaction mixture was concentrated to 1 mL in a 3 kDa molecular weight cutoff spin concentrator, diluted to 20 mL with milliQ water to reduce the salt concentration, and then concentrated back to 1 mL and lyophilized. The lyophilized reaction mixture was then denatured overnight in 7 M guanidium chloride, 50 mM Tris pH 7.5, 2 mM EDTA and ran on a Zorbax 300SB-C18 (9.4×250 mm, 5 m, Agilent) column. After 5 minutes at 10% acetontrile with 0.1% TFA in 0.1% aqueous TFA, the acetonitrile concentration was increased to 30% over 5 minutes, and then to 50% over 20 minutes. Aβ(M1-37-GGGRR [SEQ ID NO: 7]) eluted at 17.5 minutes. Aβ(M1-37-GGGRR [SEQ ID NO: 7]) identity was confirmed by LC/MS (m/z=4689.85 observed, 4688.30 expected) and the fraction containing it was lyophilized and stored at −20° C. for later use.

### ThT Assay

Aβ peptides were denatured and SEC-isolated in 20 mM sodium phosphate pH 8.0. Concentrations were determined by A275 and stock solutions of 20.2 μM peptide in elution buffer were prepared. To 990 μL of each stock solution was added 10 μL of thioflavin T (2 mM in water), yielding 1 mL of 20 μM peptide and 20 μM ThT. 20 μM ThT in elution buffer was used as diluent to make 10 μM peptide samples. Samples were aliquoted 120 μL per well to a sterile Nunc 96 well black polystyrene plate (Thermo Scientific, Cat. #237105). A Molecular Devices plate reader was used to follow change in fluorescence (435 ex/480 em) over 48-60 hours.

### Negative Contrast Transmission Electron Microscopy of Aβ Fibrils

Samples of Aβ(M1-37-GGGRR [SEQ ID NO: 7]) (n=6) and Aβ(M1-42) (n=2) lacking ThT were included alongside ThT containing samples in the assay described above. Following aggregation, these samples were applied to carbon-coated Formvar grids, left for 1 minute, fixed with glutaraldehyde, washed with MQ water, and wicked dry with filter paper. 2% uranyl acetate was then added and incubated for two minutes. The grid was wicked dry and allowed to air dry for 10 minutes. Grids were then stored in a sealed container and viewed under a Tencai G2 BIOTWIN electron microscope operated at 80 kV.

### Initial Evolution of SrtA to Recognize Aβ

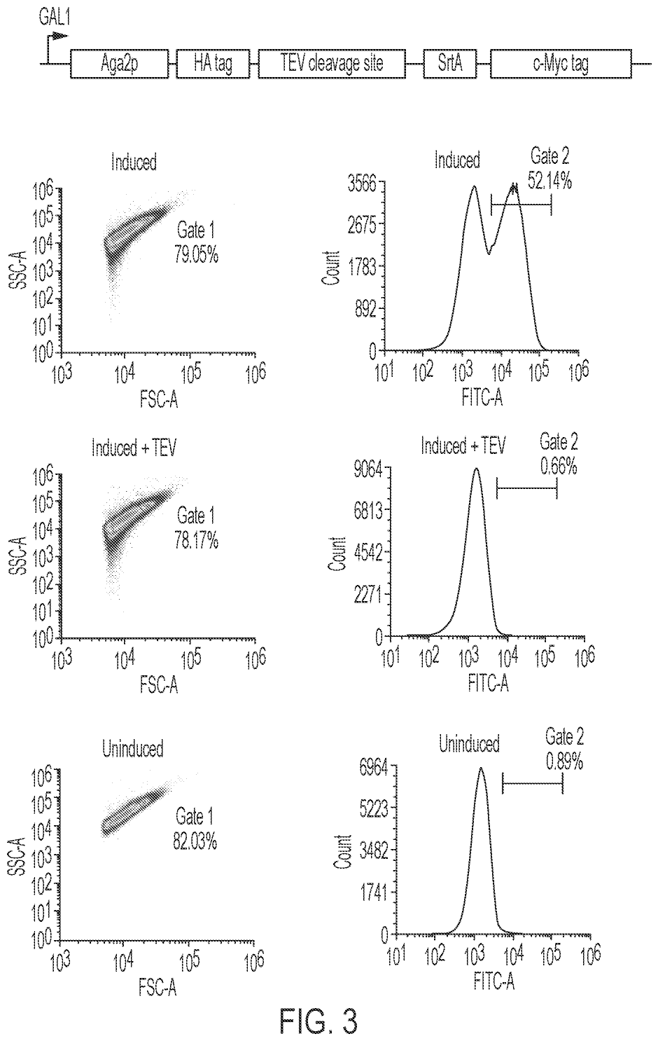

This example describes evolution of SrtA variants that modify Aβ using yeast display and fluorescence-activated cell sorting (FACS) (FIG. 2). Briefly, yeast display a library of sortase variants conjugated to triglycine peptides with N-termini that are free for sortase-catalyzed reactions. The library is then incubated with an N-terminally biotinylated target substrate and non-biotinylated off-target substrates. Sortase variants that catalyze transpeptidation between triglycine and the target substrate biotinylate the surfaces of the yeast cells that encode them. Activity on off-target substrates by promiscuous sortase variants leads to reduced biotinylation of the cells that encode them. After removal of cell surface-displayed sortases with TEV protease (FIG. 3), cells are stained with fluorophore-linked streptavidin and the biotinylated cells encoding active and selective sortase variants are isolated by FACS.

Evolution was started from a library of sortase variants previously evolved to recognize LPESG [SEQ ID NO: 4] substrates (library 4S.6). Given that the target sequence, LMVGG [SEQ ID NO: 3], deviates from the wild-type sorting sequence, LPXTG [SEQ ID NO: 104], at the second and fourth positions, it was reasoned that mutants already possessing altered substrate recognition at the fourth position were a more promising starting point than wild-type SrtA. This starting pool was diversified by error-prone PCR to create the round 1 library of 4.8×107 variants. To identify variants that preferred glycine over serine at the fourth position, biotinylated LPVGG [SEQ ID NO: 12] (Btn-LPVGG [SEQ ID NO: 12]) was used as an initial positive selection substrate. The stringency of the screen was gradually increased by decreasing the Btn-LPVGG [SEQ ID NO: 12] concentration and increasing the off-target non-biotinylated LPESG [SEQ ID NO: 4] substrate concentration (Table 2). Individual clones were isolated after five cycles of enrichment. Prominent mutations from round 1 included R94P, S118I G134R, and V189F (Table 3).

TABLE 2
Evolutionary history of SrtAB. Library size at the beginning of each round,
number of sorts before re-diversification, and information on screening stringency are
provided. Changes in substrate concentrations and reaction times over the course of a round
are indicated where applicable. The sequences in each substrate relevant to sortase
recognition are in bold. Changes in incubation time over the course of a round
is indicated by an arrow where applicable. TBS-BC = 100 mM Tris pH 7.5,
500 mM NaCl, 1% BSA, 5 mM CaCl₂. PC = human plasma, 5 mM CaCl2.

| | Library | # | Positive | | Negative | Conc. | Time | |
|---|---|---|---|---|---|---|---|---|
| Round | Size | Sorts | Substrate | Conc. (nM) | Substrate | (μM) | (min) | Buffer |
| 1 | 4.8 × 10⁷ | 5 | Btn-GLPVGGV | 3200 → 100 | GLPESGT | 0 → 10 | 60 | TBS- |
| | | | [SEQ ID NO: 94] | | [SEQ ID NO: 96] | | | BC |
| 2 | 7.2 × 10⁷ | 5 | Btn-GLMVGGV | 10000 → 1000 | LMVTGV | 0 → 100 | 60 | TBS- |
| | | | [SEQ ID NO: 95] | | [SEQ ID NO: 97] | 0 → 100 | | BC |
| | | | | | LPVGGV | | | |
| | | | | | [SEQ ID NO: 98] | | | |
| 3 | 3.5 × 10⁷ | 4 | Btn-GLMVGGV | 1000 → 320 | LMVTGV | 1 → 20 | 60 | TBS- |
| | | | [SEQ ID NO: 95] | | [SEQ ID NO: 97] | 1 → 20 | | BC |
| | | | | | LPVGGV | | | |
| | | | | | [SEQ ID NO: 98] | | | |
| 4 | 1.4 × 10⁷ | 4 | Btn-GLMVGGV | 500 → 200 | LMVTGV | 20 → 100 | 60 | TBS- |
| | | | [SEQ ID NO: 95] | | [SEQ ID NO: 97] | 20 → 100 | | BC |
| | | | | | LPVGGV | | | |
| | | | | | [SEQ ID NO: 98] | | | |
| 5 | 4.2 × 10⁷ | 4 | Btn-GLMVGGV | 500 → 50 | LMVTGV | 100 | 60 | TBS- |
| | | | [SEQ ID NO: 95] | | [SEQ ID NO: 97] | 100 | | BC |
| | | | | | LPVGGV | | | |
| | | | | | [SEQ ID NO: 98] | | | |
| 6 | 4 × 10⁷ | 2 | Btn-GLMVGGV | 100 | LMVTGV | 100 | 60 | TBS- |
| | | | [SEQ ID NO: 95] | | [SEQ ID NO: 97] | 100 | | BC |
| | | | | | LPVGGV | | | |
| | | | | | [SEQ ID NO: 98] | | | |
| 7 | 2.5 × 10⁷ | 2 | Btn-GLMVGGV | 100 → 50 | LMVTGV | 100 | 60 | TBS- |
| | | | [SEQ ID NO: 95] | | [SEQ ID NO: 97] | 100 | | BC |
| | | | | | LPVGGV | | | |
| | | | | | [SEQ ID NO: 98] | | | |
| 8 | 1 × 10⁷ | 5 | Btn-GLMVGGV | 500 → 50 | LMVTGV | 100 | 60 | TBS- |
| | | | [SEQ ID NO: 95] | | [SEQ ID NO: 97] | 100 | | BC |
| | | | | | LPVGGV | | | |
| | | | | | [SEQ ID NO: 98] | | | |
| 9 | 7 × 10⁷ | 6 | Btn-GLMVGGV | 50 | ALAVGGS | 10 → 50 | | TBS- |
| | | | [SEQ ID NO: 95] | | [SEQ ID NO: 99] | 10 → 50 | 60 → 30 | BC |
| | | | | | ALPPAGS | 100 | | |
| | | | | | [SEQ ID NO: 100] | | | |
| | | | | | LPVGGV | | | |
| | | | | | [SEQ ID NO: 101] | | | |
| 10 | 2 × 10⁷ | 5 | Btn-GLMVGGV | 5000 → 500 | | - | 60 | PC |
| | | | [SEQ ID NO: 95] | | | | | |
| 11 | 8 × 10⁶ | 5 | Btn-GLMVGGV | 500 → 50 | - | - | 60 | PC |
| | | | [SEQ ID NO: 95] | | | | | |
| 12 | 2 × 10⁷ | 5 | Btn-GLMVGGV | 50 | - | - | 60 → 20 | PC |
| | | | [SEQ ID NO: 95] | | | | | |
| 13 | 5 × 10⁷ | 6 | Btn-GLMVGGV | 50 → 30 | - | - | 20 | PC |
| | | | [SEQ ID NO: 95] | | | | | |
| 14 | 4.4 × 10⁷ | 6 | Btn-GLMVGGV | 30 | - | - | 20 | PC |
| | | | [SEQ ID NO: 95] | | | | | |
| 15 | 1 × 10⁷ | 5 | Btn-Aβ40 | 200 → 20 | - | - | 60 → 15 | PC |
| 16 | 1.2 × 10⁷ | 6 | Btn-Aβ40Btn- | 10 → 5 | - | - | 15 | PC |
| | | | Aβ42 | 10 → 5 | | | | |

TABLE 3
Mutations in representative clones from rounds 1-16 are shown relative to
the evolutionary starting sequence, 4S.6 [SEQ ID NO: 2]. These clones
were the most abundant sequences in the evolving pool at the end of their
rounds, with the exceptions of clone 8.5-H3 and SrtAβ, which were identified
by single clone FACS at the end of round 8 and round 16, respectively.

| 4S.6 | R1 | R2 | R4 | R5 | R7 | R8 | 8.5-H3 | R9 | R10 | R11 | R12 | R13 | SrtAβ |
|---|---|---|---|---|---|---|---|---|---|---|---|---|---|
| K62 | | | | | | R | R | R | R | R | R | R | |
| A73 | | | | | | | | | | V | V | | |
| I76 | | | | | | L | | L | L | L | L | L | L |
| R94 | P | Y | Y | Y | Y | | | | | | | | |
| S102 | | | | | | | | | | | | | C |
| E105 | | | | | | | | | | | | D | D |
| N107 | | | | | | | D | D | D | D | D | D | D |
| S118 | I | I | I | I | I | I | I | I | I | I | I | I | I |
| A122 | | W | | | | | | | | | | | |
| I123 | | | | L | L | L | L | L | L | L | L | L | L |
| D124 | | G | L | L | L | L | L | L | L | L | L | L | L |
| N127 | | | | | | Y | | | Y | H | H | H | H |
| G134 | R | R | R | R | R | R | R | R | R | R | R | R | R |
| K138 | | | I | I | I | L | I | I | L | L | L | L | L |
| G139 | | | | | | | | | | | | | D |
| M141 | | | | | | | | | | | | | I |
| K145 | | | | | | T | | | T | T | T | T | T |
| G147 | | | | | | | | | | | | C | |
| N148 | | | | | | | | | | | S | | |
| K152 | | | | | | | | | | | | | R |
| M155 | | | | | | | | | | I | I | I | I |
| S157 | | | | | | | | R | | | | | |
| R159 | | | | | | C | | H | C | C | C | C | C |
| K162 | | | | | | | | | | | | | R |
| D170 | | | | | | | | | | E | E | E | |
| Q172 | | | | | | | | | H | H | H | H | H |
| K173 | | | | | E | | E | E | E | E | E | E | E |
| K177 | | | | | | | R | R | | | | | R |
| V182 | | | A | A | A | A | A | A | A | A | A | A | A |
| V189 | F | F | F | F | F | F | I | I | F | F | F | F | Y |
| T196 | | | S | S | S | S | S | S | S | S | S | S | S |
| R197 | | | S | S | S | S | S | S | S | S | S | S | S |
| K206 | | | | | | | | | E | E | | | R |

The pool was re-diversified by error-prone PCR to create the round 2 library. This library showed sufficient activity on Btn-LMVGG[SEQ ID NO: 3] to permit FACS using this substrate. As in round 1, stringency was increased by reducing the amount of positive selection substrate while increasing the amounts of negative selection substrates, in this case LPVGG [SEQ ID NO: 12] and LMVTG [SEQ ID NO: 11] (Table 2). After round 2, it was observed that the S118I, G134R, and V189F mutations from round 1 had persisted, but that the identity of residue 94 was diverse (Tyr, Leu, Arg, Pro, His, or Gln) among sequenced clones. In addition, ˜98% of sequenced clones had mutations at residue 124 (Asp to Gly, Leu, or Tyr).

Rounds 3 through 7 consisted of iterative cycles of diversification by error-prone PCR and FACS screening for activity on Btn-LMVGG [SEQ ID NO: 3] with progressively higher stringencies (Table 2). At the end of round 3, a clone that represented 3.5% of the population and contained new mutations K138I, V182A, T196S, and R197S, in addition to previously observed mutations R94Y, S118I, D124L, G134R, and V189F was identified. By the end of round 4 this clone represented 74% of the population (Table 3), indicating a substantial fitness benefit from some combination of K138I, V182A, T196S, and R197S.

The most common sequence emerging from round 5 (36% of the population) was the round 4 consensus sequence plus an I123L mutation. I123L was the most common new mutation emerging in round 5, present in 67% of sequenced clones. Notably, the V182A, T196S, and R197S mutations that first appeared at the end of round 3 reached 100% prevalence in the population. Following two additional rounds of diversification and sorting, the consensus sequence of the round 7 pool (29% of the population) contained R94Y, S118I, I123L, D124L, G134R, K138I, K173E, V182A, V189F, T196S, and R197S (Table 3). Analysis of previous sequencing data showed that this clone first appeared at the end of round 5, where it made up 9% of the population.

Of these 11 mutations, V182A, T196S, and R197S were particularly interesting because of their early prevalence. Additionally, mutations at residues 182 and 196 were previously observed in SrtA variants with improved activity or single-position altered substrate recognition, while residue 197 is a crucial part of the active site in wild-type SrtA32-35. The round 8 library was generated using site-saturation mutagenesis at these three positions followed by error-prone PCR. Mutations V182A, T196S, and R197S remained fixed in sequences emerging from round 8, confirming the fitness advantage afforded by these three mutations. Additional well-represented mutations that appeared in round 8 include K62R (present in 84% of sequenced clones), I76L (60%), the reversion mutation Y94R (62%), N107D (20%), N127Y (15%), I138L (15%), K145T (15%), M155I (20%), R159C (15%), K173E (57%), K177R (24%), and F189I (35%).

### SrtA Evolution in Human Plasma

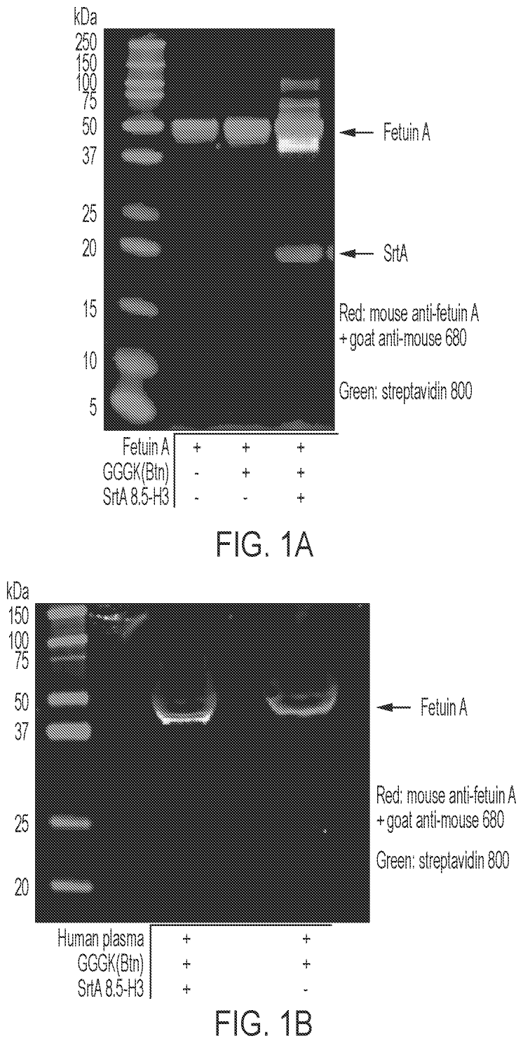

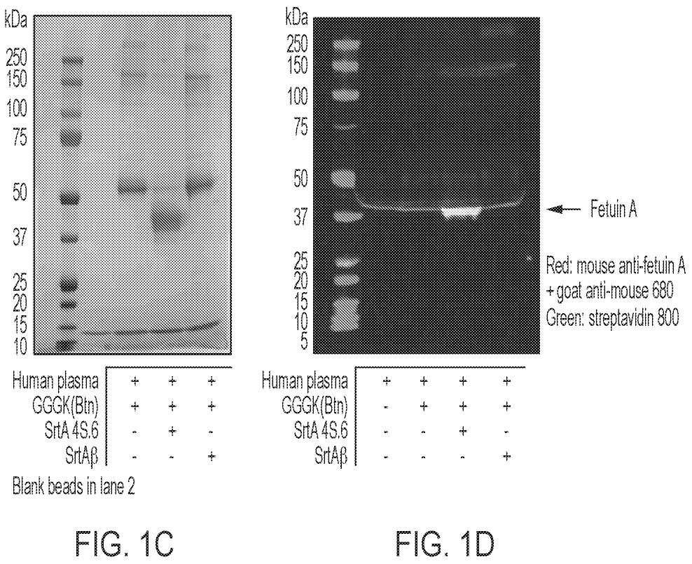

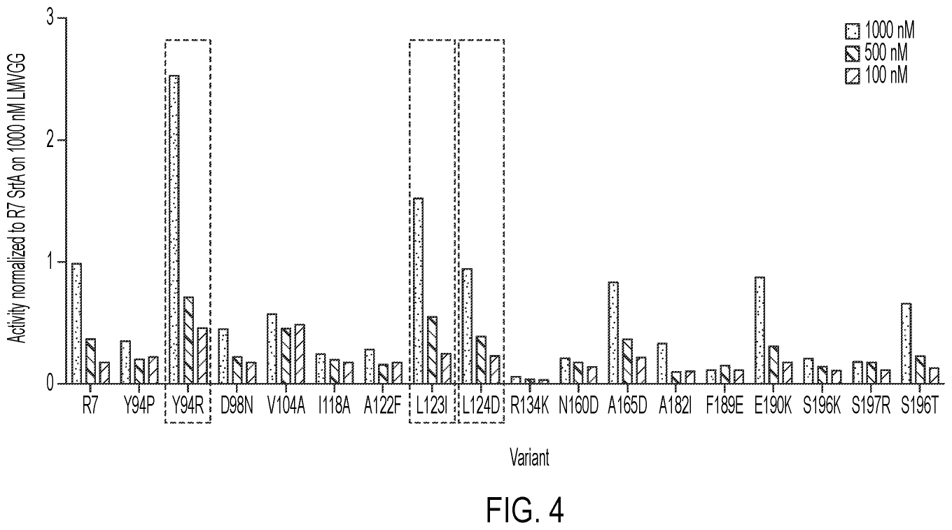

While the above screens for sortase activity on LMVGG [SEQ ID NO: 3] were conducted in TBS buffer, the goal of modifying Aβ in endogenous contexts requires that the evolved enzyme be active in biological fluids. It has been observed that sortase enzymes evolved for LPESG [SEQ ID NO: 4] recognition—including clone 4S.6, the starting point of this study—are capable of modifying fetuin A in human plasma, presumably through its native LPPAG [SEQ ID NO: 5] sequence. Indeed, a 4-fold molar excess of a round 8 clone also supported labeling of purified fetuin A in DPBS (FIG. 1A), and overnight incubation of human plasma with 50 μM evolved sortase and 1 mM GGGK[SEQ ID NO: 6](Btn) also led to fetuin A labeling (FIG. 1B). To evolve decreased recognition of fetuin A, additional rounds of evolution were conducted with negative selection against the LPPAG [SEQ ID NO: 5] sequence of fetuin. This negative selection was achieved by including LPPAG [SEQ ID NO: 5] peptide in the sortase reaction mixtures (round 9) and by conducting the sortase reactions directly in human plasma (rounds 10-16) Over these eight rounds of evolution, a sortase variant with greatly reduced activity on fetuin A relative to the starting sortase 4S.6 was generated (FIG. 1C). Between the end of round 8 and the beginning of round 9, the activity of a series of single-reversion mutants from the round 7 consensus sequence was investigated (FIG. 4). These data revealed the importance of mutations at residues 94, 123, and 124. As such, site-saturation mutagenesis was conducted at these three residues and adjacent residue 122, followed by error-prone PCR to generate the round 9 library. Increased off-target LPPAG [SEQ ID NO: 5] concentration and decreased reaction times were used to increase selection stringency over the course of round 9 screening (Table 2). Sequencing the pool at the end of round 9 revealed enrichment of many mutations that were first observed in round 8. This included K62R (up to 100% from 84% of sequenced clones), I76L (up to 97% from 60%), the reversion mutation Y94R (up to 91% from 62%), N107D (up to 61% from 20%), I138L (up to 42% from 15%), K145T (up to 55% from 15%), R159C or H (up to 21% and 53% from 15% and 4%, respectively), K173E (up to 85% from 57%), K177R (up to 72% from 24%), and F189I (up to 72% from 35%) (Table 3).

To maintain selection against fetuin A recognition while introducing selection against other motifs that exist in human plasma, the sortase reactions for the screens were conducted directly in human plasma from round 10 onward. 100-fold higher concentrations of Btn-LMVGG [SEQ ID NO: 3] were initially needed to observe sortase conjugation in human plasma than were needed to observe conjugation in TBS, indicating that specific labeling of the desired target is more difficult in plasma. (Table 2). Analysis of the round 10 sequencing results showed further enrichment of N107D (present in 95% of sequenced clones), N127Y (56%), M155I (35%), and K145T (98%). The Y94R reversion, I138L, R159C, and K173E all reached 100% abundance by the end of round 10. New mutations included N127H (27%) and Q172H (65%). Round 11 resulted in further enrichment of N127H (present in 75% of sequenced clones), M155I (70%), Q172H (100%), and the appearance of the G139D (28%). N172H and M155I further enriched in round 12 (both to 87% abundance), where an E105D mutation appeared in 32% of clones. By the end of round 13, E105D was found in 88% of sequenced clones.

The inability to use more stringent conditions in round 14 than in round 13 (Table 2), coupled with the low convergence of the resulting pools, prompted us to use DNA shuffling in an attempt to escape a potential fitness plateau. The round 14 pool was shuffled with the eSrtA pentamutant in a 1:1 ratio and subjected the products to error-prone PCR to create the round 15 library. In round 15, Aβ40 conjugated to biotin at its N-terminus through an aminohexanoic acid linker (Btn-LC-Aβ40) was used as the target substrate instead of Btn-LMVGG [SEQ ID NO: 3] to ensure activity on the full target peptide and not only the recognition motif. In round 16 a 1:1 mixture of Btn-LC-AP40 and Btn-LC-AP42 was used to select for activity on two different Aβ alloforms. The most notable mutation to emerge in round 16 was S102C, which was present in all four of the most active individual clones. Other mutations of note include M141I, K152R, and K206R. Given the level of activity already observed and the lack of additional strongly enriching mutations after round 13, the evolution campaign was ended and characterized the evolved sortase enzymes.

Flow cytometry analysis of the pools at the end of each screening round revealed an upward trend in activity on the LMVGG [SEQ ID NO: 3] substrate (from round 1 through round 9). An initial downward trend in pool activity was observed upon switching to human plasma as reaction buffer. This downward trend was reversed in round 12, but activity dropped and plateaued again in rounds 13 and 14. The noticeable increase in activity from round 14 to round 15 both in TBS and in plasma (43% increase in TBS, 54% in plasma) indicates that DNA shuffling was a successful strategy to escape an apparent fitness plateau, and the overall trend in strongly increased activity between round 1 and round 16 confirmed a successful evolutionary campaign for a SrtA variant with activity on Aβ.

### Characterization of Mutants and Mutational Analysis

At the end of round 8, individual variants were isolated by sorting single cells into a 96-well plate. The activity of 32 evolved sortase clones towards LMVGG [SEQ ID NO: 3] and LPVGG [SEQ ID NO: 12] was assessed by a flow cytometry assay. Clone 8.5-H3 demonstrated the best combination of activity on LMVGG [SEQ ID NO: 3] and selectivity over LPVGG [SEQ ID NO: 12]. When expressed and purified, this variant was active on LMVGG [SEQ ID NO: 3] in an established HPLC assay for SrtA activity, converting 10% of M substrate to product in two hours, an improvement from previous rounds. Kinetic parameters for 8.5-H3 were determined to be kcat=0.012 s−1 (95% CI=0.009 to 0.017 s−1) to with KM=52 μM (95% CI=29 to 103 μM) using an established fluorescence assay. Western blot analysis revealed that variant 8.5-H3 was able to conjugate a variety of Aβ isoforms with GGGK[SEQ ID NO: 6](Btn), demonstrating that sortases evolved to process LMVGG [SEQ ID NO: 3] also show activity on Aβ.

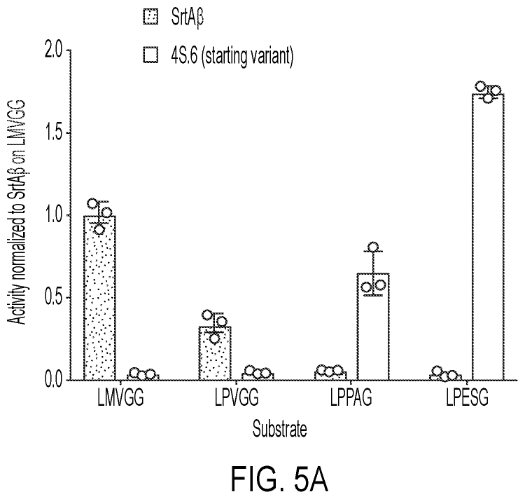

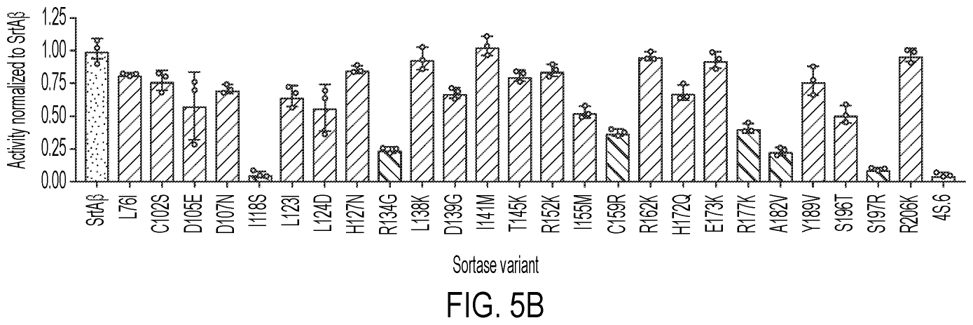

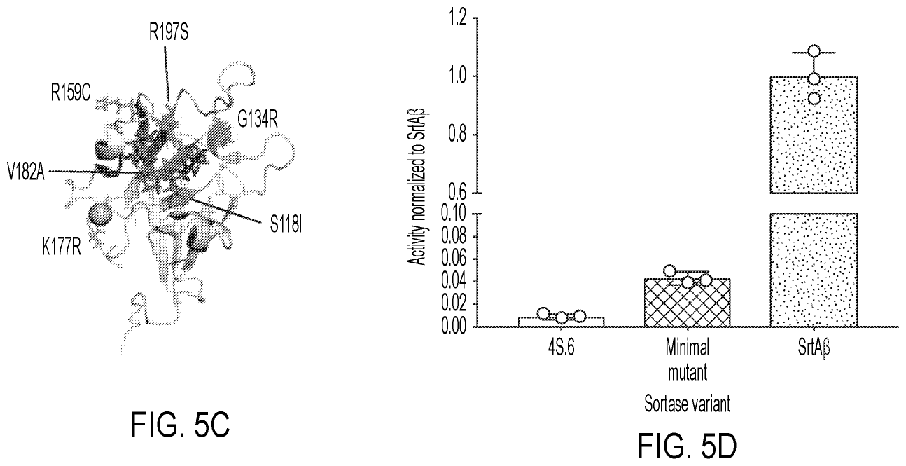

Individual variants from round 16 were sorted and re-assayed for LMVGG [SEQ ID NO: 3] activity at the end of the round. The top variant from round 16 (SrtAβ) was assayed by flow cytometry on a panel of substrates, which revealed a greatly altered substrate profile from the starting enzyme 4S.6 (FIG. 5A). The results were consistent with positive selection for activity on LPVGG [SEQ ID NO: 12] in round 1 and LMVGG [SEQ ID NO: 3] in subsequent rounds with negative selection against LPESG [SEQ ID NO: 4] in round 1 and against LPPAG [SEQ ID NO: 5] in round 9-16. Compared to the starting enzyme 4S.6, SrtAβ has 53-fold reduced activity on LPESG [SEQ ID NO: 4], 11-fold reduced activity on LPPAG [SEQ ID NO: 5], and 28-fold increased activity on LMVGG [SEQ ID NO: 3] (FIG. 5A). SrtAβ has a 30-fold preference for LMVGG [SEQ ID NO: 3] over LPESG [SEQ ID NO: 4], whereas 4S.6 has a 49-fold preference for LPESG [SEQ ID NO: 4] over LMVGG [SEQ ID NO: 3]. Overall, SrtAβ evolved a 1,470-fold change in preference to favor LMVGG [SEQ ID NO: 3] over LPESG [SEQ ID NO: 4].

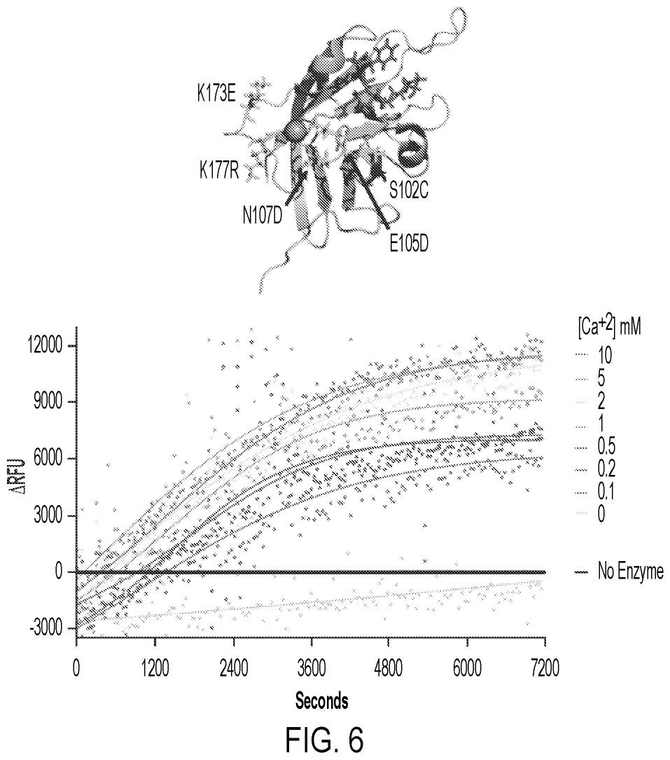

SrtAβ contains 25 mutations relative to the starting sortase enzyme 4S.6. To determine the relative importance of individual mutations to activity on LMVGG [SEQ ID NO: 3], each mutation was reverted back to its corresponding residue in the starting enzyme. These 25 single-mutant variants were assayed alongside SrtAβ and 4S.6 by flow cytometry (FIG. 5B). Eleven of the reversions reduced enzyme activity by less than 25%, eight reduced activity between 25-50%, and six reduced enzyme activity by at least two-fold. The six mutations are shown in FIG. 5C. Notably, reversion mutations at residues 118 and 197 reduced activity on the LMVGG [SEQ ID NO: 3] substrate greater than 90%, near the low level of activity demonstrated by the starting enzyme 4S.6. Two of these six mutations are at residues that were identified as modulators of sortase substrate specificity in the previous evolution campaigns (residues 118 and 182), but the remaining four were at novel residues. Notably, three of these four novel residues are outside of the substrate binding pocket, and the fourth, R197, is highly conserved across the sortase superfamily. It has been observed that R197 in wild-type S. aureus SrtA stabilizes the binding of the LPXTG [SEQ ID NO: 104] sorting signal or the oxyanion intermediates generated during catalysis. That a non-conservative mutation at this residue is not only tolerated, but required, is surprising. These results highlight the challenge of a priori prediction of mutations that alter SrtA specificity, and the importance of including random mutagenesis as a diversification strategy (FIG. 5D). A minimal mutant containing these six mutations in the 4S.6 background showed a 4-fold improvement in LMVGG [SEQ ID NO: 3] activity relative to 4S.6, but 23-fold lower activity than SrtAβ (FIG. 5D). This result confirms that other mutations, though less important individually, collectively contribute to substantially improved target activity. Four of these other mutations, in addition to R177K, are at residues located near the calcium binding site in the wild-type enzyme. Assaying SrtAβ activity at various calcium concentrations revealed compatibility with a broad range of concentrations (0.1 to 10 mM) that include physiological calcium concentrations, but confirmed that calcium is still required for activity (FIG. 6).

To confirm that the shift in substrate specificity observed in the flow cytometry assay translated to purified enzymes, the kinetic parameters of 4S.6 and SrtAβ on LPESG [SEQ ID NO: 4] and LMVGG [SEQ ID NO: 3] were determined using a HPLC assay. Sortase 4S.6 showed kcat=0.36 s−1 (95% CI=0.22 to 0.96 s−1) and KM=610 μM (95% CI=90 to 5550 μM) on LPESG [SEQ ID NO: 4], whereas SrtAβ activity on LPESG [SEQ ID NO: 4] was too low to establish accurate kinetic parameters. SrtAβ had kcat=0.018 s−1 (95% CI=0.015 to 0.023 s−1) and KM=128 μM (95% CI=87 to 198 μM) on LMVGG [SEQ ID NO: 3], whereas 4S.6 activity on LMVGG [SEQ ID NO: 3] was not detectable. These findings confirm that the evolution resulted in a large change in substrate preference, consistent with the >1,400-fold change observed in flow cytometry assays (FIGS. 5A-5D).

To obtain a more quantitative understanding of the evolved enzyme's activity on Aβ40 in plasma, an ELISA to measure biotinylated Aβ was developed. Streptavidin was used to capture biotinylated peptide and detection accomplished using 4G8, a monoclonal antibody that recognizes Aβ residues 17-24. Aβ40 labeled with GGGK[SEQ ID NO: 6](Btn) was used as the calibrant. Employing this assay, SrtAβ activity on Aβ40 spiked into human plasma was confirmed, with 1.5 μM SrtAβ generating 2.3 μM of biotinylated product from 5 μM Aβ40 in two hours. Increasing the amount of GGGK[SEQ ID NO: 6](Btn) nucleophile greatly improved reaction yields.

Concentrations of Aβ peptides are important biomarkers of Alzheimer's disease. This is especially true of Aβ42 in CSF, where a decrease to roughly 50% of baseline Aβ42 levels is typically observed in AD patients. To enable labeling and detection of physiologically relevant amounts of Aβ, the format of the ELISA was changed to capture the product with monoclonal antibody m266 (the epitope of which spans Aβ residues 13-26) and detect with streptavidin-HRP. After optimizing the concentrations of various assay components, Aβ-Btn conjugates were detected and quantified at concentrations comparable to commercial Aβ ELISA kits (Table 4). The lower limit of quantitation (LLoQ) is defined as the lowest standard with a signal higher than the average signal of the blank samples plus nine standard deviations, and allows a percent recovery of 80-120%. In six runs over six days, LLoQ for the assay was 39-78 pg/mL, or roughly 10-20 μM. Using this SrtAβ-mediated assay, labeling of Aβ40 spiked into human plasma at concentrations as low as 5 nM was observed. Given that typical Aβ concentrations in human CSF are on a similar order of magnitude, these observations indicate the possibility of using SrtAβ to label endogenous Aβ in CSF, where the generation, clearance, and aggregation of Aβ are all intimately connected with AD etiology.

TABLE 4
Comparison of developed ELISA to commercial kits. Searching publicly available databases
and manufacturer's catalogs for ELISA kits that detect human Aβ reveals kits designed
to be used with standards ranging in concentration from 7.8 to 100,000 pg/mL, with most
individual kits ranging from ~10 to 1000 pg/mL. Most commercially available kits are
designed for the detection of a single Aβ alloform, normally Aβ40 or Aβ42. In
contrast, SrtAβ has been shown to modify Aβ40, Aβ42, and Aβ43. The performance
of these kits can be measured in multiple ways. The lower limit of quantitation (LLoQ)
is defined as the lowest standard concentration with a signal higher than the average signal
of the blank samples plus nine standard deviations that allows a percent recovery of 80-
120%. The lower limit of detection (LLoD) is defined as the lowest standard concentration
with a signal higher than the average signal of the blank samples plus nine standard deviations.
The minimum detectable dose (MDD), referred to as sensitivity by some manufacturers, is
the lowest concentration of analyte that can be differentiated from zero. It is obtained
by taking the average of the blanks, adding 2 (MDD + 2) or 3 (MDD + 3) standard
deviations, and using that value to calculate a concentration. In six ELISA experiments
on six separate days, LLoQ = 39 pg/mL was observed on five occasions and LLoQ =
78 pg/mL was observed once. LLoD = 39 pg/mL was observed on all six occasions. The
average MDD across assays was 5.5 or 7.2 pg/mL, depending on whether 2 or 3 standard deviations
were added to the average of the blanks.

| | Alloform(s) | Standard range | LLoQ | LLoD | MDD + 3 | MDD + 2 |
|---|---|---|---|---|---|---|
| Source | detected | (pg/mL) | (pg/mL) | (pg/mL) | (pg/mL) | (pg/mL) |
| This Work | 40 and longer | 39-2500 | 39-78 | 39 | 7.2 | 5.5 |
| LifeSpan BioSciences | 40 | 12.4-1000 | — | — | <4.6 | — |
| LifeSpan BioSciences | 42 | 15.6-1000 | — | — | <9.4 | — |
| RayBiotech | 40 | 100-100000 | — | — | — | 100 |
| Biomatik | 40 | 12.4-1000 | — | — | — | 4.6 |
| Biomatik | 42 | 12.4-1000 | — | — | — | 5 |
| R&D Systems | 40 | 15.6-1000 | — | — | — | 4 |
| R&D Systems | 42 | 7.8-500 | — | — | — | 2.3 |
| Biorbyt | 40 | 125-8000 | — | — | 31.2 | — |
| Biorbyt | 42 | 312-20000 | — | — | 78 | — |
| IBL International | 40 | 188-1880 | — | — | 104 | — |
| IBL International | 42 | 7.8-125 | 28.6 | 16 | — | — |
| Abexa | 40 | 15.6-1000 | — | — | 9.4 | — |
| Abexa | 42 | 15.6-1000 | — | — | 9.4 | — |
| Thermo | 40 | 7.8-500 | — | — | — | <6 |
| Thermo | 42 | 15.6-1000 | — | — | — | <10 |
| Novus | 40 | 15.6-1000 | — | — | 9.4 | — |
| Novus | 42 | 15.6-1000 | — | — | 9.4 | — |

### SrtAβ Labels Endogenous Aβ in CSF

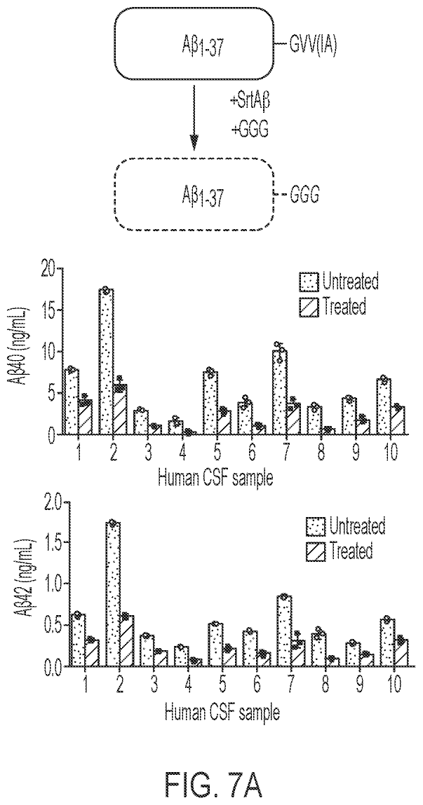

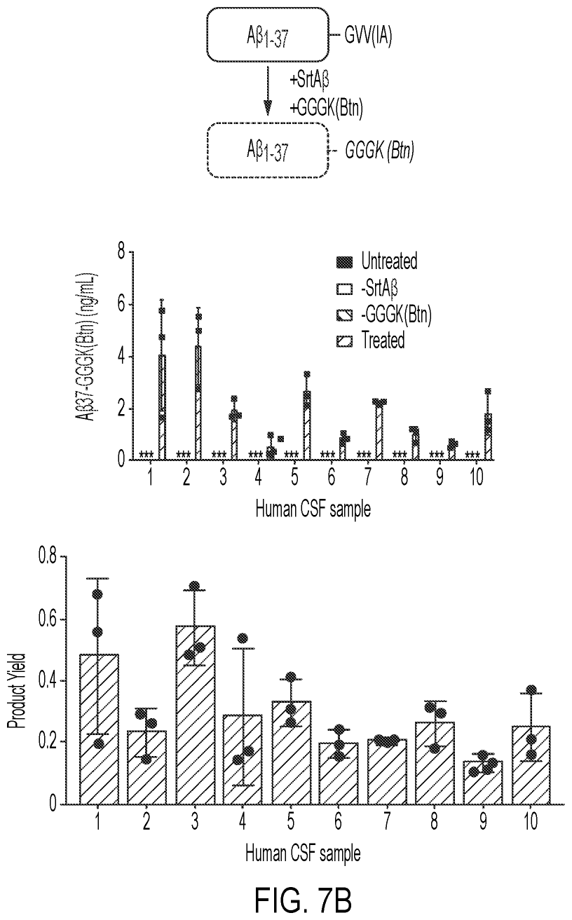

The ability to site-specifically modify endogenous Aβ in CSF would provide researchers with new ways to interrogate or influence these dynamic processes. Labeling of endogenous Aβ in CSF was demonstrated. First, Aβ levels in CSF samples were measured using immunoassays specific for Aβ terminating at Val40 or Ala4240. Because sortase-mediated conjugation of Aβ40 and Aβ42 destroys the C-terminal epitopes used for immunodetection, it was reasoned that this reaction would cause a loss of ELISA-measured signal. Indeed, after treating the samples with SrtAβ and GGG, losses in signal ranging from 47% to 77% were observed, confirming that the enzyme was active in CSF (FIG. 7A).

While this loss of signal is consistent with transpeptidation, it might also be explained by hydrolysis or interference of the sortase enzyme with the binding of the detection antibody. Loss of signal due to aggregation is unlikely since it has previously been observed that incubation of biological samples at room temperature for up to 24 hours does not alter detection of Aβ4241. Besides the enzyme and GGG, the only difference between treated and untreated samples was the addition of calcium, a cation known to influence in vitro aggregation of Aβ42. However, CSF already contains micromolar levels of calcium and it is unlikely that a modest increase in calcium would induce aggregation of Aβ present at nanomolar concentrations.

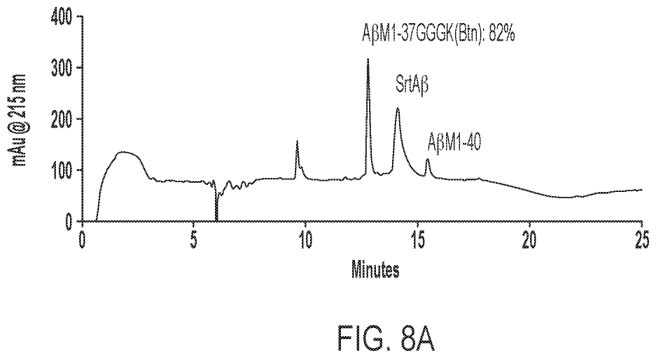

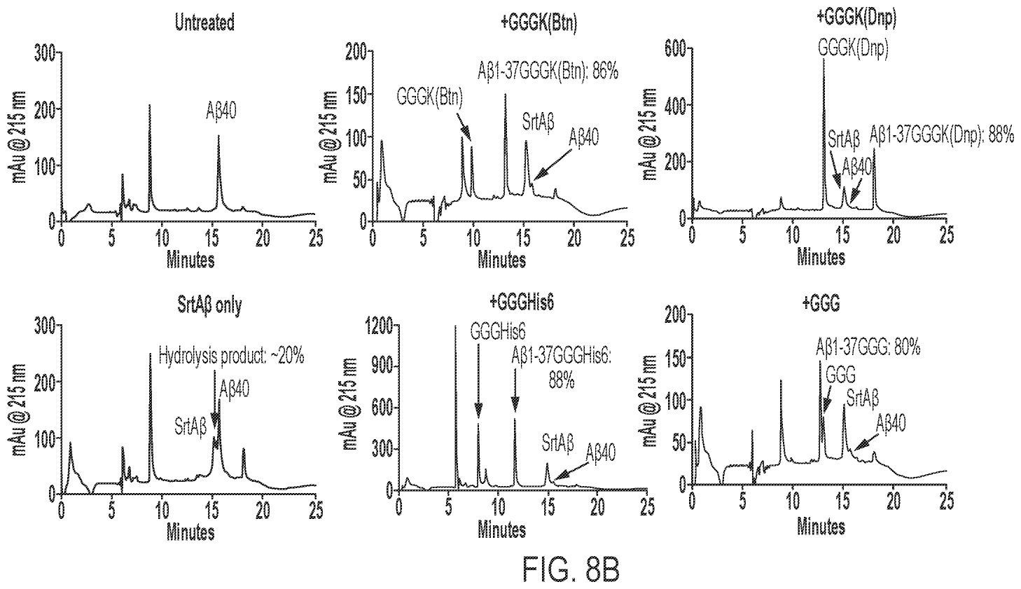

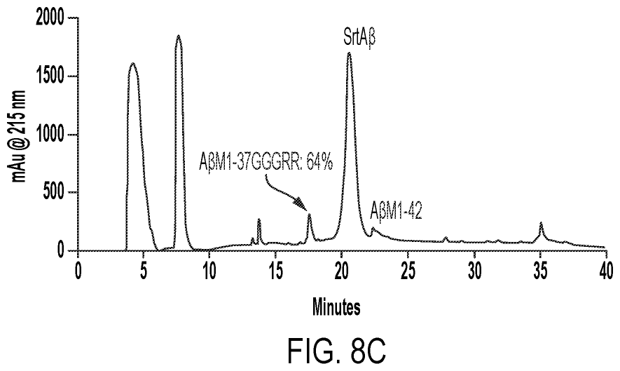

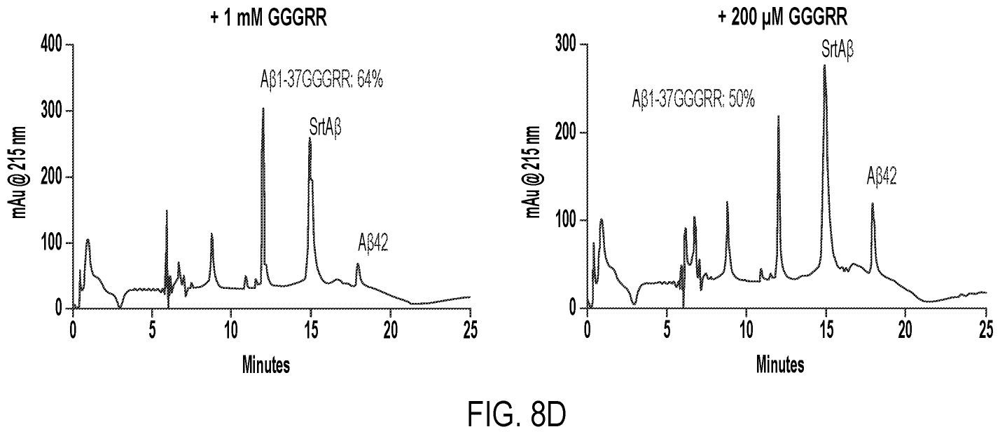

To obtain a more direct read out of transpeptidation activity, AβM1-37-GGGK[SEQ ID NO: 6](Btn) was produced semi-synthetically (FIG. 8A), and used as a standard to detect reaction product generated by SrtAβ-catalyzed conjugation with GGGK[SEQ ID NO: 6](Btn). As before, Aβ peptides were captured using the m266 antibody and detected via streptavidin-HRP. Aβ labeling efficiencies of 13% to 56% were observed (FIG. 7B). These efficiencies are lower than those observed with GGG labeling. The lower efficiency is not likely due to SrtAβ preferring GGG over GGGK[SEQ ID NO: 6](Btn), since reactions of chemically synthesized Aβ with equimolar amounts of different triglycine nucleophiles yield similar amounts of transpeptidation products (FIG. 8B). These data demonstrate the ability of the evolved SrtAβ enzyme to modify endogenous Aβ in human CSF.

### Sortagging Aβ42 Alters Aggregation Kinetics

Next, Aβ was conjugated to a molecule that would impede its aggregation. Previous studies showed that the hydrophobic C-terminus of Aβ42 is well-resolved in the NMR solution structure of Aβ42 fibrils. Replacement of hydrophobic C-terminal residues with more hydrophilic residues would alter the aggregation propensity of the resulting peptides. To test this possibility, Aβ42 was expressed and purified. Immediately following batch purification, a portion of the recombinant Aβ42 (20 mL of ˜40 μM) was treated overnight with 20 μM SrtAβ and 200 μM GGGRR [SEQ ID NO: 7]. Transpeptidation should replace the last five residues of Aβ(M1-42), GVVIA [SEQ ID NO: 101], with GGGRR [SEQ ID NO: 7], yielding a more hydrophilic 43-mer. Aβ(M1-37-GGGRR [SEQ ID NO: 7]), the identity of which was confirmed by mass spectrometry, eluted from reverse-phase HPLC before AβM1-42 (FIG. 8C). The aggregation propensity of the HPLC-isolated Aβ(M1-37-GGRR [SEQ ID NO: 102]) was then compared to that of recombinant AβM1-42 from the same initial batch purification.

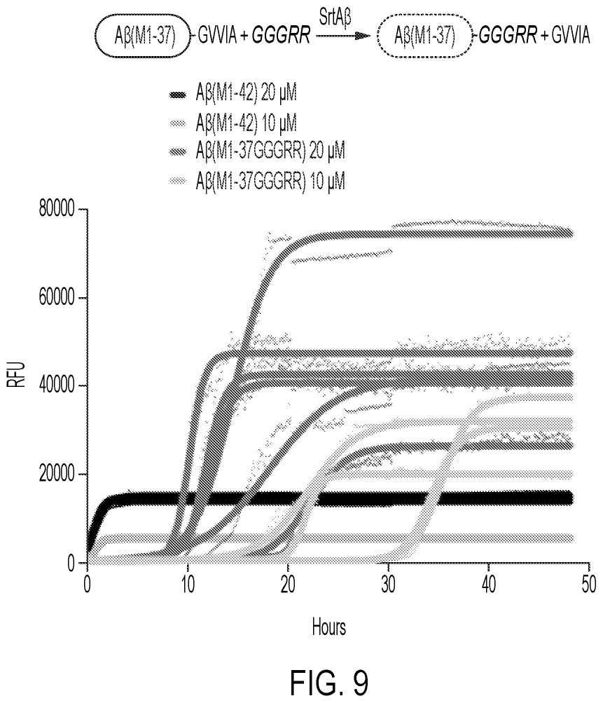

Using a continuous thioflavin T (ThT) binding assay45, the SrtAβ-modified peptides were found to take much longer to nucleate into aggregates. The lag time to initiation of detectable aggregation for 20 μM Aβ(M1-42) occurred within 5 minutes, whereas the lag time for 20 μM Aβ(M1-37GGGRR [SEQ ID NO: 7]) was 8.2 hours. The modified peptides also took ˜40-fold longer to reach half maximal aggregation (0.70 or 0.56 hours for 10 or 20 μM AβM1-42 versus 28 or 14.6 hours for 10 or 20 μM Aβ(M1-37GGGRR [SEQ ID NO: 7])) (FIG. 9). The impaired aggregation of SrtAβ-modified AβM1-42 was replicated with recombinant Aβ(M1-37GGGRR[SEQ ID NO: 7]). In contrast to the delayed kinetics of aggregation, the maximum ThT signals of C-terminally modified fibrils were higher (46,000 vs 14,000 RFU at 20 μM, 30,000 vs 5,000 RFU at 10 μM). Thus, while the lag time for AβM1-37GGGRR [SEQ ID NO: 7] was much longer than for AβM1-42, the rate of aggregation and the extent of ThT binding was greater for AβM1-37GGGRR[SEQ ID NO: 7]. These results indicate that modification of the Aβ C-terminus delays nucleation, but once nuclei are formed elongation is rapid and the structure formed binds ThT in a manner distinct from Aβ42. Indeed, EM analysis of aggregation end-products revealed significant ultrastructural differences in the fibrils formed by AβM1-37GGGRR [SEQ ID NO: 7] and AβM1-42. Collectively, these results establish the modification of a disease-associated form of Aβ to a form less prone to aggregation by transpeptidation using a laboratory-evolved sortase enzyme.

REPRESENTATIVE SEQUENCES
>Wild-type Sortase A
[SEQ ID NO: 1]
MKKWTNRLMTIAGVVLILVAAYLFAKPHIDNYLHDKDKDE
KIEQYDKNVKEQASKDKKQQAKPQIPKDKSKVAGYIEIPD
ADIKEPVYPGPATPEQLNRGVSFAEENESLDDQNISIAGH
TFIDRPNYQFTNLKAAKKGSMVYFKVGNETRKYKMTSIRD
VKPTDVEVLDEQKGKDKQLTLITCDDYNEKTGVWEKRKIF
VATEVK
>eSrtA4S.6
[SEQ ID NO: 2]
MKKWTNRLMTIAGVVLILVAAYLFAKPHIDNYLHDKDKDE
KIEQYDKNVKEQASKDKKQQAKPQIPKDKSKVAGYIEIPD
ADIKEPVYPGPATREQLDRGVSFVEENESLDDQNISISGH
TAIDRPNYQFTNLGAAKKGSMVYFKVGNETRKYKMTSIRN
VKPTAVEVLDEQKGKDKQLTLVTCDDYNVETGVWETRKIF
VATEVK
>SrtAB
[SEQ ID NO: 8]
MKKWTNRLMTIAGVVLILVAAYLFAKPHIDNYLHDKDKDE
KIEQYDKNVKEQASKDKKQQAKPQIPKDKSKVAGYLEIPD
ADIKEPVYPGPATREQLDRGVCFVDEDESLDDQNISIIGH
TALLRPHYQFTNLRAAKLDSIVYFTVGNETRRYKITSICN
VRPTAVEVLDEHEGKDRQLTLATCDDYNYETGVWESSKIF
VATEVR

### EQUIVALENTS AND SCOPE

Those skilled in the art will recognize, or be able to ascertain using no more than routine experimentation, many equivalents to the specific embodiments of the invention described herein. The scope of the present invention is not intended to be limited to the above description, but rather is as set forth in the appended claims.

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention also includes embodiments in which more than one, or all of the group members are present in, employed in, or otherwise relevant to a given product or process.

Furthermore, it is to be understood that the invention encompasses all variations, combinations, and permutations in which one or more limitations, elements, clauses, descriptive terms, etc., from one or more of the claims or from relevant portions of the description is introduced into another claim. For example, any claim that is dependent on another claim can be modified to include one or more limitations found in any other claim that is dependent on the same base claim. Furthermore, where the claims recite a composition, it is to be understood that methods of using the composition for any of the purposes disclosed herein are included, and methods of making the composition according to any of the methods of making disclosed herein or other methods known in the art are included, unless otherwise indicated or unless it would be evident to one of ordinary skill in the art that a contradiction or inconsistency would arise.

Where elements are presented as lists, e.g., in Markush group format, it is to be understood that each subgroup of the elements is also disclosed, and any element(s) can be removed from the group. It is also noted that the term “comprising” is intended to be open and permits the inclusion of additional elements or steps. It should be understood that, in general, where the invention, or aspects of the invention, is/are referred to as comprising particular elements, features, steps, etc., certain embodiments of the invention or aspects of the invention consist, or consist essentially of, such elements, features, steps, etc. For purposes of simplicity those embodiments have not been specifically set forth in haec verba herein. Thus for each embodiment of the invention that comprises one or more elements, features, steps, etc., the invention also provides embodiments that consist or consist essentially of those elements, features, steps, etc.

Where ranges are given, endpoints are included. Furthermore, it is to be understood that unless otherwise indicated or otherwise evident from the context and/or the understanding of one of ordinary skill in the art, values that are expressed as ranges can assume any specific value within the stated ranges in different embodiments of the invention, to the tenth of the unit of the lower limit of the range, unless the context clearly dictates otherwise. It is also to be understood that unless otherwise indicated or otherwise evident from the context and/or the understanding of one of ordinary skill in the art, values expressed as ranges can assume any subrange within the given range, wherein the endpoints of the subrange are expressed to the same degree of accuracy as the tenth of the unit of the lower limit of the range.

In addition, it is to be understood that any particular embodiment of the present invention may be explicitly excluded from any one or more of the claims. Where ranges are given, any value within the range may explicitly be excluded from any one or more of the claims. Any embodiment, element, feature, application, or aspect of the compositions and/or methods of the invention, can be excluded from any one or more claims. For purposes of brevity, all of the embodiments in which one or more elements, features, purposes, or aspects is excluded are not set forth explicitly herein.

What is claimed is:

1. A sortase that binds substrates comprising the amino acid sequence LMVGG [SEQ ID NO: 3], wherein the sortase comprises an amino acid sequence that is at least 80% identical to the amino acid sequence provided in SEQ ID NO: 2, or a fragment thereof, wherein the amino acid sequence of the sortase includes one or more substitutions selected from the group consisting of the amino acid substitutions listed in Table 3, relative to SEQ ID NO 2.

2. The sortase of claim 1, wherein the sortase comprises at least three mutations, at least four mutations, at least five, at least six, at least seven, at least eight, at least nine, or at least 10 amino acid substitutions as compared to the amino acid sequence set forth in SEQ ID NO: 2 or a fragment thereof.

3. The sortase of claim 1 or 2, wherein the sortase comprises amino acid substitutions at two or more of the following positions: I76, S102, E105, N107, S118, I123, D124, N127, G134, K138, G139, M141, K145, K152, M155, R159, K162, Q172, K173, K177, V182, V189, T196, R197, and K206, relative to SEQ ID NO: 2.

4. The sortase of any one of claims 1 to 3, wherein the sortase comprises the following amino acid substitutions relative to SEQ ID NO: 2: S118I, G134R, R159C, K177R, V182A, and R197S.

5. The sortase of any one of claims 1 to 3, wherein the sortase comprises the following amino acid substitutions relative to SEQ ID NO: 2: I76L, S102C, E105D, N107D, S118I, I123L, D124L, N127H, G134R, K138L, G139D, M141I, K145T, K152R, M155I, R159C, K162R, Q172H, K173E, K177R, V182A, V189Y, T196S, R197S, and K206R.

6. The sortase of any one of claims 1 to 5, wherein the sortase has reduced selectivity for peptides having the amino acid sequence LPPAG [SEQ ID NO: 5] relative to the sortase set forth in SEQ ID NO: 2.

7. The sortase of any one of claims 1 to 6, wherein the sortase has increased selectivity for peptides having the amino acid sequence LMVGG [SEQ ID NO: 3] relative to the sortase set forth in SEQ ID NO: 2.

8. The sortase of any one of claims 1 to 7, wherein the sortase has 10-fold to 100-fold preference for LMVGG [SEQ ID NO: 3] over LPESG [SEQ ID NO: 4].

9. The sortase of any one of claims 1 to 8, wherein the sortase has a change in substrate preference of at least 1,400-fold to favor LMVGG [SEQ ID NO: 3] over LPESG [SEQ ID NO: 4].

10. The sortase of any one of claims 1 to 9, wherein the sortase modifies an Alzheimer's disease-associated amyloid β-protein (Aβ).

11. The sortase of claim 10, wherein the modifying comprises conjugating a heterologous peptide to the amyloid β-protein (Aβ).

12. The sortase of claim 10 or claim 11, wherein the amyloid β-protein (Aβ) comprises between 30 and 51 amino acids.

13. The sortase of any one of claims 10 to 12, wherein the amyloid β-protein (Aβ) comprises between 40 and 42 amino acids.

14. The sortase of any one of claims 1 to 13, wherein the sortase is active in human plasma.

15. A method for producing a sortase protein variant, the method comprising:
(a) expressing in a population of yeast cells one or more fusion proteins, each fusion protein comprising a sortase protein or portion thereof conjugated to a triglycine peptide having an N-terminus capable of reacting in sortase-catalyzed reactions;
(b) incubating the yeast cell population of (a) with a mixture comprising N-terminally biotinylated target substrates and non-biotinylated off-target substrates under conditions under which the sortases expressed by the yeast catalyze transpeptidation of the biotinylated target substrates to the surface of the yeast cells;
(c) treating the yeast cells with a TEV protease;
(d) incubating the cells with fluorescently-labeled streptavidin under conditions under which the streptavidin binds to the biotin on the surface of the yeast cells comprising the target substrate; and
(e) isolating the fluorescently-labeled yeast cells form the population of yeast cells using fluorescence-activated cell sorting (FACS).

16. The method of claim 15, wherein the sortase of the fusion protein of (a) comprises the amino acid sequence set forth in SEQ ID NO: 2.

17. The method of claim 15 or 16, wherein the target substrate comprises the amino acid sequence LMVGG [SEQ ID NO: 3].

18. The method of any one of claims 15 to 17, wherein the incubating occurs in human plasma.

19. A method for detecting a target protein in a biological sample, the method comprising
(a) contacting a biological sample with the sortase of any one of claims 1 to 14, and a probe comprising:
(i) one or more detectable agents; and
(ii) a peptide comprising the amino acid sequence GGGK [SEQ ID NO: 6] under conditions under which the sortase conjugates the one or more detectable agents to the target protein;
(b) removing unconjugated probe from the biological sample; and
(c) detecting the presence of the detectable agent conjugated to the target protein.

20. The method of claim 19, wherein the target protein comprises the amino acid sequence LMVGG [SEQ ID NO: 3].

21. The method of claim 19 or 20, wherein the target protein is amyloid β-protein (Aβ).

22. The method of any one of claims 15 to 17, wherein the biological sample comprises cerebrospinal fluid (CSF).

23. The method of any one of claims 15 to 18, wherein the detectable agent comprises biotin.

24. The method of claim 23, wherein the biotin comprises a fluorescent label.

25. A method for inhibiting amyloid β-protein (Aβ) aggregation or plaque formation in a cell or subject, the method comprising administering to the cell or subject the sortase of any one of claims 1 to 14 and a peptide comprising the amino acid sequence GGGRR [SEQ ID NO: 7].

26. The method of claim 25, wherein the GGGRR [SEQ ID NO: 7] is at the N-terminus of the peptide.

27. The method of claim 25 or 26, wherein the cell is a human cell, or the subject is a human.

28. The method of any one of claims 25 to 27, wherein the cell is a central nervous system cell, optionally wherein the cell is a neuron.

29. The method of any one of claims 25 to 28, wherein the subject has or is suspected of having Alzheimer's disease.

30. The sortase of any one of claims 25 to 29, wherein the amyloid β-protein (Aβ) comprises between 30 and 51 amino acids.

31. The sortase of claim 30, wherein the amyloid β-protein (Aβ) comprises between 40 and 42 amino acids.

32. A method for treating or ameliorating Alzheimer's disease (AD) in a subject, the method comprising administering to a subject having AD the sortase of any one of claims 1 to 14 and a peptide comprising the amino acid sequence GGGRR [SEQ ID NO: 7].

33. The method of claim 32, wherein the subject is a human.

34. The method of claim 32 or 33, wherein the GGGRR [SEQ ID NO: 7] is at the N-terminus of the peptide.

35. The method of any one of claims 32 to 34, wherein Aβ aggregation or plaque formation in a cell is inhibited.
